(19) United States
(12) Patent Application Publication
(10) Pub. No.: US 2024/0293486 A1
(43) Pub. Date: Sep. 5, 2024

(54) Compositions For and Methods of Promoting Respiratory Health

(71) Applicants: Duke University, Durham (NC); Ann and Robert H. Lurie Children's Hospital of Chicago, Chicago, IL (US)

(72) Inventors: Matthew Kelly, Durham, NC (US); Patrick Seed, Chicago, IL (US)

(21) Appl. No.: 18/575,412

(22) PCT Filed: Jul. 1, 2022
(86) PCT No.: PCT/US22/73367

§ 371 Date: Dec. 29, 2023

Related U.S. Application Data
(60) Provisional application No. 63/217,384, filed on Jul. 1, 2021.

(51) Int. Cl. A61K 35/747; A61K 45/06; A61P 31/04; C12Q 1/689

(52) U.S. Cl. CPC A61K 35/747; A61K 45/06; A61P 31/04; C12Q 1/689

(57) ABSTRACT

Disclosed herein are biotherapeutics comprising probiotics or a consortium of probiotics. Disclosed herein are biotherapeutics comprising factors secreted from probiotics or factors secreted from a consortium of probiotics. Disclosed herein are also pharmaceutical formulations comprising a disclosed biotherapeutic. Also disclosed are methods of promoting respiratory health, reducing the risk of developing and infection, treating and/or preventing a bacterial colonization and/or infection, and modulating microbial diversity and/or composition.

### CROSS-REFERENCE TO RELATED APPLICATIONS

This application claims priority to U.S. Provisional Application No. 63/217,384 filed 1 Jul. 2021, which is incorporated herein in its entirety.

### STATEMENT REGARDING FEDERALLY SPONSORED RESEARCH OR DEVELOPMENT

This invention was made with government support under P30-AI045008, P30-AI064518, K23-AI135090, 7R01-GM108494, D43-TW009337, and P30-AI045008 awarded by the National Institutes of Health. The government has certain rights in the invention.

### REFERENCE TO THE SEQUENCE LISTING

The Sequence Listing submitted 1 Jul. 2022 as a text file named “22-2046-WO_Sequence_Listing”, created on 1 Jul. 2022 and having a size of 26 kilobytes is hereby incorporated by reference pursuant to 37 C.F.R. § 1.52(e)(5).

IV. BACKGROUND

Pneumonia is the leading infectious killer of children, accounting for more than 800,000 child deaths each year. Streptococcus pneumoniae (pneumococcus) is a leading cause of severe infections among children and adults. Streptococcus pneumoniae (Sp) causes most fatal cases of childhood pneumonia despite vaccination. Colonization of the nasopharynx (NP) precedes infections by Sp, and the NP microbiota serves as a barrier to pathogen colonization. Interactions between commensal microbes in the upper respiratory tract and S. pneumoniae are poorly described.

There is thus an urgent need to develop alternative approaches to preventing infections—particularly respiratory infections—caused by S. pneumoniae, H. influenzae, or Moraxella catarrhalis.

### BRIEF DESCRIPTION OF THE FIGURES

FIG. 1A-FIG. 1B show alpha diversity of the nasopharyngeal microbiome among mother-infant dyads in Botswana. Box plots depict nasopharyngeal microbiome diversity, as measured by the Shannon index (FIG. 1A), and richness, as measured by the number of unique ASVs (FIG. 1B). Maternal nasopharyngeal samples are from the birth visit only (M0), and are shown in red, while nasopharyngeal samples from infants were sequenced at ten time points during the first year of life (I0-I12) and are shown in blue. The Shannon index of the infant nasopharyngeal microbiome is higher at birth than at all later time points (Wilcoxon signed-rank tests, p<0.0001) and compared to maternal samples (Wilcoxon signed-rank tests, p<0.0001). The number of unique ASVs in infant nasopharyngeal samples is lower than in maternal samples from birth through five months of age (Wilcoxon signed-rank tests, p<0.05).

FIG. 2A-FIG. 2B show the composition of the nasopharyngeal microbiome among mother-infant dyads in Botswana. FIG. 2A shows the principal components (PCoA) plot based on Bray-Curtis distances showing nasopharyngeal microbiome composition among mothers at delivery (M0) and infants throughout the first year of life (I0-I12). During infancy, nasopharyngeal microbiome composition progressively diverges from the composition of the adult nasopharyngeal microbiome. Ellipses define the regions containing 80% of all samples that can be drawn from the underlying multivariate t distribution. Ellipses are shown for samples from mothers at delivery (M0), infants at birth (I0), and infants at 12 months of age (I12). FIG. 2B shows the relative abundances of highly abundant genera in nasopharyngeal samples from mothers (M0; n=172 samples) and infants (I0-I12, n=1,368 samples) by month of visit.

FIG. 3A-FIG. 3C shows state transitions of the nasopharyngeal microbiome during infancy. Each nasopharyngeal sample was classified based on the dominant bacterial genus in that sample or, if no genus occupied ≥50% of the sequencing reads, the sample was classified as biodiverse. Each diagram depicts changes in infant nasopharyngeal microbiome biotype between two consecutive study visits. FIG. 3A shows months 0 to 1, months 1 to 2, and months 2 to 3. FIG. 3B shows months 3 to 4, months 4 to 5, and months 5 to 6. FIG. 3C shows months 6 to 8, months 8 to 10, and months 10 to 12. Arrows point to the directionality of microbiome transitions while ribbon widths represent the frequency of these transitions. The colors correspond to specific microbiome biotypes.

FIG. 4A-FIG. 4D show associations between environmental exposures and the composition of the nasopharyngeal microbiome during infancy. MaAsLin2 was used to fit log-transformed generalized linear mixed models evaluating associations between sociodemographic factors and environmental exposures and the relative abundances of bacterial genera within the infant nasopharyngeal microbiome. The coefficients from these models, which correspond to the relative effect sizes of associations, are shown for significant associations (q<0.20) identified for antibiotic exposures (FIG. 4A), the number of PCV-13 doses (FIG. 4B), breastfeeding (FIG. 4C), and the winter season (FIG. 4D). The number of PCV-13 doses was modeled as an ordinal variable such that the coefficients represent the relative effect sizes associated with each successive vaccine dose. Bacterial genera that increase in relative abundance with the exposure are shown as yellow bars. Bacterial genera that decrease in relative abundance with the exposure are shown as blue bars.

FIG. 5A-FIG. 5C show strain-specific inhibition of pneumococcal growth by Corynebacterium. Two strains of S. pneumoniae, one reference strain (ATCC 6303; serotype 3) and one strain isolated from an infant nasopharyngeal sample (05-160; serotype 11A) were separately added to sterile, cell-free media from overnight cultures of Corynebacterium strains. Growth of these strains of S. pneumoniae was determined by OD600 readings that were normalized to blank media controls (TSB and BHIT-TSB). FIG. 5A-FIG. 5C show growth curves of S. pneumoniae strains in different media, including cell-free media from inhibitory strains of C. accolens (05-122) (FIG. 5A), C. tuberculostearicum (05-144) (FIG. 5B), and C. coyleae (05-104) (FIG. 5C). FIG. 5D-FIG. 5F show growth curves of S. pneumoniae strains in different media, including cell-free media from strains of C. accolens (05-161) (FIG. 5D), C. tuberculostearicum (05-150) (FIG. 5D), and C. propinquum (05-124) (FIG. 5F) that do not demonstrate pneumococcal growth inhibition. The decline in OD600 observed in these experiments after the peak density is reached reflects a transition of S. pneumoniae from the exponential growth phase to the autolysis phase. TSB, tryptic soy broth; BHIT-TSB, brain heart infusion medium with 0.2% Tween80 diluted 50% with tryptic soy broth.

FIG. 6 shows the cumulative incidence curve for S. pneumoniae colonization among infants in Botswana. S. pneumoniae colonization was assessed through quantitative PCR testing of infant nasopharyngeal samples collected monthly (0-6 months) or bimonthly (6-12 months) during the first year of life.

FIG. 7 shows children with a Corynebacterium-dominant biotype have lower risk of Sp colonization. Forest plots of Sp colonization risk by nasopharyngeal microbiome biotype. Corynebacterium-dominant biotype has a lower hazard of Sp colonization (hazard ratio: 0.39, 95% CI: 0.20-0.76, p=0.005).

FIG. 8 shows the adherence of C. accolens 05-122 (C122) and competition with Spa 05-160 (S160) on A549 respiratory epithelium cells. A549 monolayers at confluency were infected for 30 min at a multiplicity of infection (MOI) of 20:1 in a 96 well moderate throughput format using strains C122 or S160. Non-adherent bacteria removed with three PBS washes. After the final washing, adherent bacteria were liberated from the epithelial cells and plated for CFU. The left panel shows the percentage of adherent C122 and S160. For adherence competition (C122+S160), A549 were first incubated with C122 for 30 min, were washed, exposed to S160 for 30 min at an MOI of 20:1, and then wash again. The remaining adherent bacteria were liberated and count for CFU on media to exclusively culture Sp. Monolayers were monitored throughout the assays for retention of cells and confluency. Non-parametric tests were performed with FDR-correction. Data represent 3 biological repeats with technical triplicates. Panels on the right show oil-immersion 100× magnification of bacterial adherence to A549 cells.

FIG. 9A-FIG. 9B shows Corynebacterium spp. species and strain-specific secretion of Sp growth inhibitors. Pneumococcal growth was determined by OD600 readings normalized to blank media controls. FIG. 9A shows growth curves of S. pneumoniae strains in different media, including cell-free media from an inhibitory strain of C. tuberculostearicum (05-144). FIG. 9B shows growth curves of S. pneumoniae strains in different media, including cell-free media from a strain of C. tuberculostearicum (05-150) that did not demonstrate pneumococcal growth inhibition. Similar inhibition was identified from C. coyleae, C. striatum, and C. accolens species with strain-dependent variation in inhibitory effects.

FIG. 10 shows the Corynebacterium pan-genome. 273 publicly available complete genomes for Corynebacterium spp. from NCBI were obtained. Anvio was used to create the display.

FIG. 11 shows adherence and invasion assays. The left side shows the measurement of adherence of Corynebacterium strains to respiratory epithelium and right side shows assessment of inhibition of Sp adherence and invasion by Corynebacterium strains.

FIG. 12A-FIG. 12D shows the secretome assays. FIG. 12A shows secretion of factors by Corynebacterium strains grown in liquid media. FIG. 12B shows the assessment of Sp growth in the presence of supernatants from strain cultures. FIG. 12C shows the secretion of factors by host respiratory epithelium in response to contact with Corynebacterium strains while FIG. 12D shows assessment of Sp growth in the presence of supernatants from Corynebacterium-exposed respiratory epithelium.

FIG. 13 shows power calculations. Power curves are shown for various sample and effect sizes using a pairwise model. All comparative analyses are anticipated to have at least 50 isolate pairs, indicating that >80% detects an effect size of 0.1.

### BRIEF SUMMARY

Disclosed herein is a pharmaceutical formulation comprising a probiotic, and at least one pharmaceutically acceptable carrier. Disclosed herein is a pharmaceutical formulation comprising a consortium of probiotics, and at least one pharmaceutically acceptable carrier. Disclosed herein is a pharmaceutical formulation comprising factors secreted by a probiotic, and at least one pharmaceutically acceptable carrier. Disclosed herein is a pharmaceutical formulation comprising factors secreted by a consortium of probiotics, and at least one pharmaceutically acceptable carrier. Disclosed herein is a pharmaceutical formulation comprising at least one strain from the bacterial genus Corynebacterium. Disclosed herein is a pharmaceutical formulation comprising one or more of at least one strain from the bacterial genus Corynebacterium, at least one strain from the bacterial genus Dolosigranulum, at least one strain from the bacterial genus Streptococcus, and at least one strain from the bacterial genus Lactobacillus.

Disclosed herein is a repository of microbiome sequence data generated by one or more methods disclosed here.

Disclosed herein is a method of promoting respiratory health in a subject, the method comprising administering to a subject in need thereof a therapeutically effective amount of a biotherapeutic, wherein the biotherapeutic inhibits and/or prevents the growth of one or more pathogenic bacteria in one or more parts of the subject's respiratory system. Disclosed herein is a method of promoting respiratory health in a subject, the method comprising administering to a subject in need thereof a therapeutically effective amount of a biotherapeutic, thereby inhibiting and/or preventing the growth of one or more pathogenic bacteria in one or more parts of the subject's respiratory system. Disclosed herein is a method of reducing the risk of developing a pathogenic bacterial infection, the method comprising administering to a subject in need thereof a therapeutically effective amount of a biotherapeutic, thereby preventing and/or inhibiting the colonization of the pathogenic bacteria. Disclosed herein is a method of reducing the risk of developing a S. pneumoniae infection, the method comprising administering to a subject in need thereof a therapeutically effective amount of a biotherapeutic, thereby preventing and/or inhibiting the colonization of S. pneumoniae. Disclosed herein is a method of treating S. pneumoniae colonization and/or infection in a subject, the method comprising administering a therapeutically effective amount of a biotherapeutic to the subject in need thereof, wherein the colonization of S. pneumoniae in the subject's respiratory system is inhibited and/or prevented following administration. Disclosed herein is a method of treating S. pneumoniae colonization in a subject, the method comprising administering a therapeutically effective amount of a biotherapeutic to the subject in need thereof, thereby inhibiting and/or preventing colonization of S. pneumoniae in the subject's respiratory system. Disclosed herein is a method of modulating microbial diversity or composition in a subject, the method comprising characterizing the microbiome of a biological sample obtained from a subject; and administering one or more biotherapeutics to the subject, wherein following administration, the microbiome is modulated.

Disclosed herein is a kit comprising at least one strain from the bacterial genus Corynebacterium. Disclosed herein is a kit comprising at least one strain from the bacterial genus Dolosigranulum. Disclosed herein is a kit comprising at least one strain from the bacterial genus Streptococcus. Disclosed herein is a kit comprising at least one strain from the bacterial genus Lactobacillus. Disclosed herein is a kit comprising at least one strain from the bacterial genus Corynebacterium sp., at least one strain from the bacterial genus Dolosigranulum sp., at least one strain from the bacterial genus Streptococcus, and at least one strain from the bacterial genus Lactobacillus. Disclosed herein is a kit comprising a pharmaceutical formulation comprising at least one strain from the bacterial genus Corynebacterium. Disclosed herein is a kit comprising a pharmaceutical formulation comprising at least one strain from the bacterial genus Dolosigranulum. Disclosed herein is a kit comprising a pharmaceutical formulation comprising at least one strain from the bacterial genus Streptococcus. Disclosed herein is a kit comprising a pharmaceutical formulation comprising at least one strain from the bacterial genus Lactobacillus. Disclosed herein is a kit comprising a pharmaceutical formulation comprising at least one strain from the bacterial genus Corynebacterium, at least one strain from the bacterial genus Dolosigranulum, at least one strain from the bacterial genus Streptococcus, and at least one strain from the bacterial genus Lactobacillus.

### DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Abbreviations

Used throughout are abbreviations, including the following: ASV (amplicon sequence variant), ATCC (American Type Culture Collection), BHI (brain heart infusion), BHIT-TSB (brain heart infusion medium with 0.2% Tween 80), CI (confidence interval), g (gram), Hib (Haemophilus influenzae type B), HR (Hazard Ratio), IPD (invasive pneumococcal disease), IQR (interquartile range), MALDI-TOF MS (matrix-assisted laser desorption ionization-time of flight mass spectrometry), OD600 (optical density measured at a wavelength of 600 nanometer), PCR (polymerase chain reaction), PCV-13 (13-valent pneumococcal conjugate vaccine), RSV (respiratory syncytial virus), rpm (revolutions per minute), rRNA (ribosomal RNA), TSB (tryptic soy broth).

### B. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is +10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a respiratory infection, be suspected of having a respiratory infection, or be at risk of developing a respiratory infection.

As used herein, the term “diagnosed” means having been subjected to an examination by a person of skill, for example, a physician, and found to have a condition that can be diagnosed or treated by one or more of the disclosed biotherapeutics, the disclosed pharmaceutical formulations, or a combination thereof, or by one or more of the disclosed methods. For example, “diagnosed with an infection” means having been subjected to an examination by a person of skill, for example, a physician, and found to have a condition (such as, for example, a respiratory infection) that can be treated by one or more of the disclosed biotherapeutics, the disclosed pharmaceutical formulations, or any combination thereof, or by one or more of the disclosed methods. For example, “suspected of having an infection” can mean having been subjected to an examination by a person of skill, for example, a physician, and found to have a condition (such as a respiratory infection) that can likely be treated by one or more of by one or more of the disclosed biotherapeutics, the disclosed pharmaceutical formulations, or any combination thereof, or by one or more of the disclosed methods. In an aspect, an examination can be physical, can involve various tests (e.g., blood tests, genotyping, biopsies, etc.), scans (e.g., CT scans, PET scans, etc.), and assays (e.g., enzymatic assay), or a combination thereof.

A “patient” refers to a subject at risk of or afflicted with an infection such as, for example, a respiratory infection. In an aspect, a patient can refer to a subject that has been diagnosed with or is suspected of having a disease or disorder such as a respiratory infection. In an aspect, a patient can refer to a subject that has been diagnosed with or is suspected of having a respiratory infection and is seeking treatment or receiving treatment for the respiratory infection). In an aspect, a patient can be a healthy subject not having an infection and not at risk of developing an infection.

“Probiotic” as used herein refers to a microorganism such as a bacteria or yeast that is associated with positive effect for and/or confers an advantage to a subject. In an aspect, a probiotic can comprise a single bacterial strain, a single bacterial strain (such as, for example, a strain of the bacterial species Corynebacterium accolens), a consortium of bacterial strains from the same genus or species, or a consortium of bacterial strains of different genera or species (e.g., Corynebacterium sp., Dolosigranulum sp., Streptococcus sp., and Lactobacillus sp.) “Probiotic” as used herein refers to factors secreted from a microorganism such as a bacteria or yeast that are associated with positive effect for and/or confer an advantage to a subject. In an aspect, a probiotic can comprise factors secreted from a single bacterial strain, a single bacterial species (such as, for example, Corynebacterium accolens), a consortium of bacterial strains from the same genus or species, or a consortium of bacterial strains from different genera or species (e.g., Corynebacterium sp., Dolosigranulum sp., Streptococcus sp., and Lactobacillus sp.)

As used herein, the term “postbiotics” refers to functional bioactive compounds produced by probiotics and can be used to promote health. The term postbiotics can be considered as a generic term for all synonyms and related terms for these microbial components. Therefore, postbiotics include metabolites, short chain fatty acids (SCFAs such as acetic acid, propionic acid, and butyric acid, etc.), microbial cell fractions, functional proteins, extracellular polysaccharides (EPS), cell lysates, acids, phenyl-lactic acid, volatile organic compounds (VOCs), B-vitamin synthesis (biotin, cobalamine, folic acid, nicotinic acid, pantothenic acid, pyridoxin, riboflavin, and thiamine), muropeptides derived from peptide glycans, antibacterial peptides (AMP) and pib types.

As used herein, the phrase “identified to be in need of treatment for a respiratory infection,” or the like, refers to selection of a subject based upon need for treatment of the respiratory infection or a subject at risk of developing a respiratory infection. For example, a subject can be identified as having a need for treatment of a respiratory infection based upon an earlier diagnosis by a person of skill and thereafter subjected to treatment for the respiratory infection. In an aspect, the identification can be performed by a person different from the person making the diagnosis. In an aspect, the administration can be performed by one who performed the diagnosis.

As used herein, “inhibit,” “inhibiting”, and “inhibition” mean to diminish or decrease an activity, level, response, condition, severity, disease, or other biological parameter. This can include, but is not limited to, the complete ablation of the activity, growth, colonization, endocytosis, spread, expression level, response, condition, severity, disease, or other biological parameter. This can also include, for example, a 10% inhibition or reduction in the activity, growth, colonization, endocytosis, spread, expression level, response, condition, severity, disease, or other biological parameter as compared to the native or control level (e.g., a subject not receiving one or more of the disclosed biotherapeutics, the disclosed pharmaceutical formulations, or a combination thereof). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to a native or control level (e.g., a subject not receiving one or more of the disclosed biotherapeutics, the disclosed pharmaceutical formulations, or a combination thereof). In an aspect, the inhibition or reduction can be 0-25%, 25-50%, 50-75%, or 75-100% as compared to native or control levels. In an aspect, a native or control level can be a pre-infection level (such as a pre-respiratory infection state).

The words “treat” or “treating” or “treatment” include palliative treatment, that is, treatment designed for the relief of symptoms rather than the curing of the infection, disease, pathological condition, or disorder; preventative treatment, that is, treatment directed to minimizing or partially or completely inhibiting the development of the associated infection, disease, pathological condition, or disorder; and supportive treatment, that is, treatment employed to supplement another specific therapy directed toward the improvement of the associated infection, disease, pathological condition, or disorder. In an aspect, the terms cover any treatment of a subject, including a mammal (e.g., a human), and includes: (i) preventing the undesired infection, physiological change, disease, pathological condition, or disorder from occurring in a subject that can be predisposed to the disease but has not yet been diagnosed as having it; (ii) inhibiting the infection, physiological change, disease, pathological condition, or disorder, i.e., arresting its development; or (iii) relieving the infection, physiological change, disease, pathological condition, or disorder, i.e., causing regression of the disease. For example, in an aspect, treating an infection can reduce the severity of an established infection in a subject by 1%-100% as compared to a control (such as, for example, an individual not having a respiratory infection). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of the infection (e.g., a respiratory infection). For example, treating an infection can reduce one or more symptoms of a disease or disorder in a subject by 1%-100% as compared to a control (such as, for example, an individual not having an infection such as a respiratory infection). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established infection. It is understood that treatment does not necessarily refer to a cure or complete ablation or eradication of an infection. However, in an aspect, treatment can refer to a cure or complete ablation or eradication of an infection (such as a respiratory infection).

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a respiratory infection is intended. The words “prevent”, “preventing”, and “prevention” also refer to prophylactic or preventative measures for protecting or precluding a subject (e.g., an individual) not having an infection (such as a respiratory infection) or related complication from progressing to that complication. In an aspect, preventing a respiratory infection is intended.

As used herein, the terms “administering” and “administration” refer to any method of providing to a subject one or more of the disclosed biotherapeutics, the disclosed pharmaceutical formulations, or any combination thereof. Such methods are well known to those skilled in the art and include, but are not limited to, the following: oral administration, transdermal administration, administration by inhalation, nasal administration or administration into other sites in the upper respiratory tract, topical administration, in utero administration, intratumoral administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can be continuous or intermittent. Administration can comprise a combination of one or more routes.

In an aspect, the skilled person can determine an efficacious dose, an efficacious schedule, and an efficacious route of administration of one or more of the disclosed biotherapeutics, the disclosed pharmaceutical formulations, or any combination thereof to treat or prevent an infection (such as a respiratory infection). In an aspect, the skilled person can also alter, change, or modify an aspect of an administering step to improve efficacy of the disclosed biotherapeutics, the disclosed pharmaceutical formulations, or any combination thereof.

By “determining the amount” is meant both an absolute quantification of a particular analyte (e.g., biomarker for an infection) or a determination of the relative abundance of a particular pathogenic and/or non-pathogenic bacteria (e.g., Corynebacterium sp. or S. pneumoniae). The phrase includes both direct or indirect measurements of abundance or both.

As used herein, the proportional abundance for each nasal bacterial taxon can be determined by (Number of sequences assigned to the taxon from the sample)/(Total number of sequences from the sample), which can be combined with nasal bacterial density to calculate taxon absolute abundance as (Proportional abundance of the taxon from the sample)×(Nasal bacterial density of the sample). In an aspect, the cultivation of specific organisms per total sample collection can also an acceptable method for calculating abundance.

As used herein, “modifying the method” can comprise modifying or changing one or more features or aspects of one or more steps of a disclosed method. In an aspect, a method can be altered by changing the amount of the disclosed biotherapeutic, the disclosed pharmaceutical formulation, or disclosed therapeutic agent administered to a subject, or by changing the frequency of administration of the disclosed biotherapeutic, the disclosed pharmaceutical formulation, or the disclosed therapeutic agent, by changing the duration of time that the disclosed biotherapeutic, the disclosed pharmaceutical formulation, or the disclosed therapeutic agent, or by substituting for one or more of the disclosed components and/or reagents with a similar or equivalent component and/or reagent. The same applies to all disclosed biotherapeutics, the disclosed pharmaceutical formulations, or any combination thereof, or all combinations thereof.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents.

Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, “concurrently” means (1) simultaneously in time, or (2) at different times during the course of a common treatment schedule.

The term “contacting” as used herein refers to bringing one or more of the disclosed biotherapeutics, the disclosed pharmaceutical formulations, or any combination thereof together with a target area or intended target area (e.g., such as an aspect of the respiratory system affected by an infection) in such a manner that the disclosed biotherapeutic, the disclosed pharmaceutical formulation, or any combination thereof can exert an effect on the intended target or targeted area either directly or indirectly. A target area or intended target area can be one or more parts of a subject's respiratory system (e.g., the subject's mouth, nose, ears, sinuses, nasopharynx or oropharynx, trachea, bronchial tubes, lungs, alveoli, bronchioles, capillaries, lung lobes, pleura, cilia, epiglottis, larynx, or any combination thereof). In an aspect, a target area or intended target area can be any cell or any organ infected by pathogenic bacteria and/or S. pneumoniae. In an aspect, a target area or intended target area can be any organ, tissue, or cells that are affected by an infection.

As used herein, “determining” can refer to measuring or ascertaining the presence and severity of an infection, such as a respiratory infection. Methods and techniques used to determine the presence and/or severity of an infection are typically known to the medical arts. For example, the art is familiar with the ways to identify and/or diagnose the presence, severity, or both of an infection (such as, for example, a respiratory infection).

As used herein, “effective amount” and “amount effective” can refer to an amount that is sufficient to achieve the desired result such as, for example, the treatment and/or prevention of an infection (e.g., a respiratory infection) or a suspected infection. As used herein, the terms “effective amount” and “amount effective” can refer to an amount that is sufficient to achieve the desired an effect on an undesired condition (e.g., a respiratory infection). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed biotherapeutic, a disclosed pharmaceutical formulation, or any combination thereof that (i) treats the particular infection, disease, condition, or disorder (e.g., a respiratory infection), (ii) attenuates, ameliorates, or eliminates one or more symptoms of the particular infection, disease, condition, or disorder (e.g., a respiratory infection), or (iii) delays the onset of one or more symptoms of the particular infection, disease, condition, or disorder described herein (e.g., a respiratory infection). The specific therapeutically effective dose level for any particular patient can depend upon a variety of factors including the disorder being treated and the severity of the infection; the disclosed biotherapeutics, the disclosed pharmaceutical formulations, or any combination thereof employed in the disclosed methods; the age, body weight, general health, sex and diet of the patient; the time of administration; the route of administration; the rate of excretion of the disclosed biotherapeutics, the disclosed pharmaceutical formulations, or any combination thereof employed; the duration of the treatment; other drugs used in combination or coincidental with the disclosed biotherapeutics, the disclosed pharmaceutical formulations, or any combination thereof, and other like factors well known in the medical arts. For example, it is well within the skill of the art to start doses of the disclosed biotherapeutics, the disclosed pharmaceutical formulations, or any combination thereof at levels lower than those required to achieve the desired therapeutic effect and to gradually increase the dosage until the desired effect is achieved. If desired, then the effective daily dose can be divided into multiple doses for purposes of administration. Consequently, a single dose of the disclosed biotherapeutics, the disclosed pharmaceutical formulations, or any combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of an infection.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of a missing, deficient, and/or mutant protein or enzyme. It should be understood that sequence with substantial sequence identity do not necessarily have the same length and may differ in length. For example, sequences that have the same nucleotide sequence but of which one has additional nucleotides on the 3′- and/or 5′-side are 100% identical.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of other therapies (e.g., other agents) includes the use of more than one therapy (e.g., drug therapy).

Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., a disclosed biotherapeutic or a disclosed pharmaceutical formulation) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., a disclosed biotherapeutic or a disclosed pharmaceutical formulation) to a subject having or diagnosed with an infection (such as, for example, a respiratory infection).

Disclosed are the components to be used to prepare the disclosed biotherapeutics and the disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### C. Compositions for Use in the Disclosed Methods

### 1. Formulations

Disclosed herein is a pharmaceutical formulation comprising one or more disclosed biotherapeutics, disclosed secreted factors, disclosed consortia of probiotics, disclosed probiotics, or any combination thereof, and at least one pharmaceutically acceptable carrier. Disclosed herein is a pharmaceutical formulation comprising biotherapeutic, and at least one pharmaceutically acceptable carrier. In an aspect, a disclosed biotherapeutic can comprise a probiotic or a consortium of probiotics. Disclosed herein is a pharmaceutical formulation comprising a probiotic, and at least one pharmaceutically acceptable carrier. Disclosed herein is a pharmaceutical formulation comprising a consortium of probiotics, and at least one pharmaceutically acceptable carrier. Disclosed herein is a pharmaceutical formulation comprising factors secreted by a probiotic, and at least one pharmaceutically acceptable carrier. Disclosed herein is a pharmaceutical formulation comprising factors secreted by a consortium of probiotics, and at least one pharmaceutically acceptable carrier.

In an aspect, a disclosed pharmaceutical formulation can further comprise a growth medium to sustain a disclosed probiotic or a disclosed consortium of probiotics prior to administration to the subject.

In an aspect of a disclosed pharmaceutical formulation, a disclosed probiotic can be lyophilized or freeze-dried. In an aspect of a disclosed pharmaceutical formulation, a disclosed consortium of probiotics can be lyophilized or freeze-dried.

In an aspect, a disclosed pharmaceutical formulation can comprise at least one lyoprotectant. In an aspect, a disclosed lyoprotectant can comprise peptone, glycerol, lactose, gelatin, glucose, sucrose, trehalose, dextran, maltodextrin, adonitol, sodium glutamate, or any combination thereof. Lyoprotectants are known to those skilled in the art.

In an aspect, a disclosed pharmaceutical formulation can comprise at least one gelling agent, preferably a pharmaceutically acceptable gelling agent. In an aspect, a disclosed pharmaceutical formulation can comprise at least preservative such as, for example, benzyl alcohol, cresols, benzoic acid, phenol, parabens, or sorbic acid. In an aspect, a disclosed pharmaceutical formulation can comprise at least one stabilizer such as, for example, a surfactant, a polymer, a polyol, a poloxamer, an albumin, a gelatin, a trehalose, a protein, a sugar, a polyvinylpyrrolidone, a N-acetyl-tryptophan (NAT), a caprylate (e.g., sodium caprylate), a polysorbate (e.g., P80), an amino acid, and a divalent metal cation (e.g., zinc).

In an aspect of a disclosed pharmaceutical formulation, a disclosed biotherapeutic can comprise one or more probiotics. In an aspect of a disclosed pharmaceutical formulation, a disclosed consortium of probiotics can comprise at least one strain from the bacterial genus Corynebacterium, at least one strain from the bacterial genus Dolosigranulum, at least one strain from the bacterial genus Streptococcus, and at least one strain from the bacterial genus Lactobacillus.

In an aspect, the at least one strain from the bacterial genus Corynebacterium can comprise one or more strains of Corynebacterium accolens. In an aspect, the at least one strain from the bacterial genus Dolosigranulum can comprise one or more strains of Dolosigranulum pigrum. In an aspect, the at least one strain from the bacterial genus Streptococcus can comprise one or more strains of Streptococcus mitis, Streptococcus salivarius, Streptococcus oralis, or Streptococcus thermophilus. In an aspect, the at least one strain from the bacterial genus Lactobacillus can comprise one or more strains of Lactobacillus rhamnosus or Lactobacillus acidophilus.

In an aspect of a disclosed pharmaceutical formulation, one or more disclosed probiotics can comprise at least one strain from the bacterial genus one Corynebacterium sp. In an aspect of a disclosed pharmaceutical formulation, one or more disclosed probiotics can comprise at least one strain from the bacterial genus Corynebacterium and at least one strain from the bacterial genus Dolosigranulum. In an aspect of a disclosed pharmaceutical formulation, one or more disclosed probiotics can comprise at least one strain from the bacterial genus Corynebacterium, at least one strain from the bacterial genus Dolosigranulum, and at least one strain from the bacterial genus Streptococcus. In an aspect of a disclosed pharmaceutical formulation, one or more disclosed probiotics can comprise at least one strain from the bacterial genus Corynebacterium, at least one strain from the bacterial genus Dolosigranulum, and at least one strain from the bacterial genus Lactobacillus. In an aspect of a disclosed pharmaceutical formulation, one or more disclosed probiotics can comprise at least one strain from the bacterial genus Corynebacterium and at least one at least one strain from the bacterial genus Streptococcus. In an aspect of a disclosed pharmaceutical formulation, one or more disclosed probiotics can comprise at least one strain from the bacterial genus Corynebacterium, at least one strain from the bacterial genus Streptococcus, and at least one strain from the bacterial genus Lactobacillus. In an aspect of a disclosed pharmaceutical formulation, one or more probiotics can comprise at least one strain from the bacterial genus Corynebacterium and at least one strain from the bacterial genus Lactobacillus. In an aspect of a disclosed pharmaceutical formulation, one or more probiotics can comprise at least one strain from the bacterial genus Corynebacterium, at least one strain from the bacterial genus Dolosigranulum, at least one strain from the bacterial genus Streptococcus, and at least one strain from the bacterial genus Lactobacillus.

In an aspect of a disclosed pharmaceutical formulation, a disclosed biotherapeutic can comprise one or more factors secreted by a disclosed probiotic or secreted by a disclosed consortium of probiotics. In an aspect of a disclosed pharmaceutical formulation, a disclosed biotherapeutic can comprise one or more factors secreted by at least one strain from the bacterial genus Corynebacterium, at least one strain from the bacterial genus Dolosigranulum, at least one strain from the bacterial genus Streptococcus, and at least one strain from the bacterial genus Lactobacillus. In an aspect of a disclosed pharmaceutical formulation, a disclosed biotherapeutic can comprise one or more factors secreted by at least one strain from the bacterial genus Corynebacterium. In an aspect of a disclosed pharmaceutical formulation, a disclosed biotherapeutic can comprise one or more factors secreted by at least one strain from the bacterial genus Corynebacterium and at least one strain from the bacterial genus Dolosigranulum. In an aspect of a disclosed pharmaceutical formulation, a disclosed biotherapeutic can comprise one or more factors secreted by at least one strain from the bacterial genus Corynebacterium, at least one strain from the bacterial genus Dolosigranulum, and at least one train from the bacterial genus Streptococcus. In an aspect of a disclosed pharmaceutical formulation, a disclosed biotherapeutic can comprise one or more factors secreted by at least one strain from the bacterial genus Corynebacterium, at least one strain from the bacterial genus Dolosigranulum, and at least one strain from the bacterial genus Lactobacillus. In an aspect of a disclosed pharmaceutical formulation, a disclosed biotherapeutic can comprise one or more factors secreted by at least one strain from the bacterial genus Corynebacterium and at least one strain from the bacterial genus Streptococcus. In an aspect of a disclosed pharmaceutical formulation, a disclosed biotherapeutic can comprise comprises one or more factors secreted by at least one strain from the bacterial genus Corynebacterium, at least one strain from the bacterial genus Streptococcus, and at least one strain from the bacterial genus Lactobacillus. In an aspect of a disclosed pharmaceutical formulation, a disclosed biotherapeutic can comprise one or more factors secreted by at least one strain from the bacterial genus Corynebacterium and at least one strain from the bacterial genus Lactobacillus.

In an aspect of a disclosed pharmaceutical formulation, a disclosed strain from the bacterial genus Corynebacterium can comprise C. accolens, C. afermentans subsp. afermentans, C. afermentans subsp. lipophilum, C. ammoniagenes, C. amycolatum, C. appendicis, C. aquaticum, C. argentoratense, C. atypicum, C. aurimucosum, C. auris, C. bovis, C. canis, C. confusum, C. coyleae, C. diphtheriae, C. durum, C. efficiens, C. equi (now Rhodococcus equi), C. falsenii, C. flavescens, C. freiburgense, C. freneyi, C. glucuronolyticum, C. glutamicum, C. granulosum, C. haemolyticum, C. halofytica, C. kroppenstedtii, C. hansenii, C. imitans, C. jeikeium (group JK), C. kroppenstedtii, C. kutscheri, C. lipophiloflavum, C. macginleyi, C. massiliense, C. mastitidis-like, C. matruchotii, C. minutissimum, C. mucifaciens, C. mycetoides, C. ovis, C. parvum (Propionibacterium acnes), C. paurometabolum, C. pilbarense, C. propinquum, C. pseudodiphtheriticum (C. hofmannii), C. pseudotuberculosis, C. pyogenes-Trueperella pyogenes, C. pyruviciproducens, C. renale, C. resistans, C. riegelii, C. simulans, C. singular, C. spec, C. sputi, C. stationis, C. striatum, C. sundsvallense, C. tenuis, C. thomsenii, C. timonense, C. tuberculostearicum, C. tuscaniense, C. ulcerans, C. urealyticum (group D2), C. urealyticum, C. xerosis, Corynebacterium_BWA136, Corynebacterium_BWA297, Corynebacterium_DU041, Corynebacterium_DU044, or any combination thereof.

In an aspect of a disclosed pharmaceutical formulation, a disclosed strain from the bacterial genus Corynebacterium can comprise C. accolens, C. coyleae, C. propinquum, C. pseudodiphtheriticum, C. amycolatum, C. glutamicum, C. aurimucosum, C. tuberculostearicum, C. afermentans, C. striatum, Corynebacterium_DU041, Corynebacterium_DU044, Corynebacterium_BWA136, Corynebacterium_BWA297, or any combination thereof.

In an aspect of a disclosed pharmaceutical formulation, a disclosed strain from the bacterial genus Corynebacterium can comprise any species identified in Table 13.

TABLE 13
Listing of Medically Relevant Corynebacterium Species.

| | Genus | |
|---|---|---|
| Family | (# species/genus) | Medically Relevant Species |
| Brevibacteriaceae | Brevibacterium (26) | B. casei, B. massiliense, B. mcbrellneri, |
| | | B. otitidis, B. paucivorans, B. |
| | | ravenspurgense, B. sanguinis |
| Cellulomonadaceae | Cellulomonas (18) | C. hominis, C. denverensis |
| Dermabacteriaceae | Brachybacterium (14) | Brachybacterium species |
| | Dermabacter (1) | D. hominis |
| | Helcobacillus (1) | H. massiliensis |
| Intrasporangiaceae | Janibacter (6) | J. melonis, Janibacter species |
| | Knoellia (3) | Knoellia species |
| Microbacteriaceae | Curtobacterium (9) | Curtobacterium species |
| | Leifsonia (15) | L. aquatica |
| | Microbacterium (73) | M. aurum, M. binotii, M. |
| | | esteraromaticum, M. foliorum, M. |
| | | hominis, M. hydrocarbonoxydans, M. |
| | | lacticum, M. laeveniformis, M. oxydans, |
| | | M. paraoxydans, M. phyllosphaerae, M. |
| | | resistans, M. schleiferi, M. |
| | | trichothecenolyticum, M. testaceum, M. |
| | | thalassium |
| | Pseudoclavibacter (3) | Pseudoclavibacter species |
| Micrococcaceae | Arthrobacter (67) | A. albus, A, aurescens, A. |
| | | creatinolyticus, A. cumminsii, A. |
| | | luteolus, A. oxydans, A. protophormiae, |
| | | A. sanguinis, A. schlerome, A. |
| | | woluwensis |
| | Rothia (6) | R. aeria, R. dentocariosa, R. |
| | | muciloginosa |
| Promicromonosporaceae | Cellulosimicrobium (3) | C. cellulans, C. funkei |
| Unassigned | Exiguobacterium (13) | E. acetylicum, E. auranticum |

In an aspect of a disclosed pharmaceutical formulation, a disclosed therapeutically effective dose can comprise a total of at least 100 bacterial CFUs of the one or more probiotics, a total of at least 200 to at least 300 bacterial CFUs of the one or more probiotics, a total of at least 300 to at least 400 bacterial CFUs of the one or more probiotics, a total of at least 400 to at least 500 bacterial CFUs of the one or more probiotics, a total of at least 500 to at least 600 bacterial CFUs of the one or more probiotics, a total of at least 600 to at least 700 bacterial CFUs of the one or more probiotics, a total of at least 700 to at least 800 bacterial CFUs of the one or more probiotics, a total of at least 800 to at least 900 bacterial CFUs of the one or more probiotics, a total of at least 900 to at least 1000 bacterial CFUs of the one or more probiotics, or a total of at least 1000 bacterial CFUs of one or more disclosed probiotics (such as, for example, at least one strain from the bacterial genus Corynebacterium, at least one strain from the bacterial genus Dolosigranulum, at least one strain from the bacterial genus Streptococcus, and at least one strain from the bacterial genus Lactobacillus, or any combination thereof).

In an aspect of a disclosed pharmaceutical formulation, a disclosed therapeutically effective dose can comprise a total of at least 1000 to at least 2000 bacterial CFUs of the one or more probiotics, a total of at least 2000 to at least 3000 bacterial CFUs of the one or more probiotics, a total of at least 3000 to at least 4000 bacterial CFUs of the one or more probiotics, a total of at least 4000 to at least 5000 bacterial CFUs of the one or more probiotics, a total of at least 5000 to at least 6000 bacterial CFUs of the one or more probiotics, a total of at least 6000 to at least 7000 bacterial CFUs of the one or more probiotics, a total of at least 8000 to at least 9000 bacterial CFUs of the one or more probiotics, or a total of at least 10,000 bacterial CFUs of one or more disclosed probiotics (such as, for example, at least one strain from the bacterial genus Corynebacterium, at least one strain from the bacterial genus Dolosigranulum, at least one strain from the bacterial genus Streptococcus, and at least one strain from the bacterial genus Lactobacillus, or any combination thereof).

In an aspect of a disclosed pharmaceutical formulation, a disclosed therapeutically effective dose can comprise a total of at least 100 bacterial CFUs of Corynebacterium sp., Dolosigranulum sp., Streptococcus sp., Lactobacillus sp., or any combination thereof, a total of at least 200 to at least 300 bacterial CFUs of Corynebacterium sp., Dolosigranulum sp., Streptococcus sp., Lactobacillus sp., or any combination thereof, a total of at least 300 to at least 400 bacterial CFUs of Corynebacterium sp., Dolosigranulum sp., Streptococcus sp., Lactobacillus sp., or any combination thereof, a total of at least 400 to at least 500 bacterial CFUs of Corynebacterium sp., Dolosigranulum sp., Streptococcus sp., Lactobacillus sp., or any combination thereof, a total of at least 500 to at least 600 bacterial CFUs of Corynebacterium sp., Dolosigranulum sp., Streptococcus sp., Lactobacillus sp., or any combination thereof, a total of at least 600 to at least 700 bacterial CFUs of Corynebacterium sp., Dolosigranulum sp., Streptococcus sp., Lactobacillus sp., or any combination thereof, a total of at least 700 to at least 800 bacterial CFUs of Corynebacterium sp., Dolosigranulum sp., Streptococcus sp., Lactobacillus sp., or any combination thereof, a total of at least 800 to at least 900 bacterial CFUs of Corynebacterium sp., Dolosigranulum sp., Streptococcus sp., Lactobacillus sp., or any combination thereof, a total of at least 900 to at least 1000 bacterial CFUs of Corynebacterium sp., Dolosigranulum sp., Streptococcus sp., Lactobacillus sp., or any combination thereof, or a total of at least 1000 bacterial CFUs of Corynebacterium sp., Dolosigranulum sp., Streptococcus sp., Lactobacillus sp., or any combination thereof.

In an aspect of a disclosed pharmaceutical formulation, a disclosed therapeutically effective dose can comprise a total of at least 1000 to at least 2000 bacterial CFUs of Corynebacterium sp., Dolosigranulum sp., Streptococcus sp., Lactobacillus sp., or any combination thereof, a total of at least 2000 to at least 3000 bacterial CFUs of Corynebacterium sp., Dolosigranulum sp., Streptococcus sp., Lactobacillus sp., or any combination thereof, a total of at least 3000 to at least 4000 bacterial CFUs of Corynebacterium sp., Dolosigranulum sp., Streptococcus sp., Lactobacillus sp., or any combination thereof, a total of at least 4000 to at least 5000 bacterial CFUs of the Corynebacterium sp., Dolosigranulum sp., Streptococcus sp., Lactobacillus sp., or any combination thereof, a total of at least 5000 to at least 6000 bacterial CFUs of Corynebacterium sp., Dolosigranulum sp., Streptococcus sp., Lactobacillus sp., or any combination thereof, a total of at least 6000 to at least 7000 bacterial CFUs of Corynebacterium sp., Dolosigranulum sp., Streptococcus sp., Lactobacillus sp., or any combination thereof, a total of at least 8000 to at least 9000 bacterial CFUs of Corynebacterium sp., Dolosigranulum sp., Streptococcus sp., Lactobacillus sp., or any combination thereof, or a total of at least 10,000 bacterial CFUs of Corynebacterium sp., Dolosigranulum sp., Streptococcus sp., Lactobacillus sp., or any combination thereof.

In an aspect, a disclosed therapeutically effective dose can comprise a total of at least 10⁴ to at least 10⁵ bacterial CFUs of the one or more probiotics, a total of at least 10⁵ to at least 10⁶ bacterial CFUs of the one or more probiotics, a total of at least 10⁶ to at least 10⁷ bacterial CFUs of the one or more probiotics, a total of at least 10⁷ to at least 10⁸ bacterial CFUs of the one or more probiotics, a total of at least 10⁸ to at least 10⁹ bacterial CFUs of the one or more probiotics, a total of at least 10⁹ to at least 10¹⁰ bacterial CFUs of the one or more probiotics, a total of at least 10¹⁰ to at least 10¹¹ bacterial CFUs of the one or more probiotics, a total of at least 10¹¹ to at least 10¹² bacterial CFUs of the one or more probiotics, a total of at least 10¹² bacterial CFUs of the one or more probiotics, or more than 10¹² bacterial CFUs of one or more disclosed probiotics (such as, for example, at least one strain from the bacterial genus Corynebacterium, at least one strain from the bacterial genus Dolosigranulum, at least one strain from the bacterial genus Streptococcus, and at least one strain from the bacterial genus Lactobacillus, or any combination thereof).

In an aspect of a disclosed pharmaceutical formulation, a disclosed therapeutically effective dose can comprise at least 10³, 10⁴, 10⁵, 10⁶, 10⁷, 10⁸, 10⁹, 10¹⁰, 10¹¹, or 10¹² CFUs of a disclosed probiotic or a consortium of disclosed probiotics, preferably at least 1.2×10³, 1.4×10³, 2×10³, or 3×10³ CFUs of a disclosed probiotic or a consortium of disclosed probiotics, at least 5×10³, 5.02×10³, 5.04×10³, 5.2×10³, or 5.4×10³ CFUs of a disclosed probiotic or a consortium of disclosed probiotics, at least 1.02×10⁴, 1.04×10⁴, 1.2×10⁴, 1.4×10⁴, 1.5×10⁴, 2×10⁴, or 3×10⁴ CFUs of a disclosed probiotic or a consortium of disclosed probiotics, at least 5×10⁴, 5.02×10⁴, 5.04×10⁴, 5.2×10⁴, or 5.4×10⁴ CFUs of a disclosed probiotic or a consortium of disclosed probiotics, at least 1.02×10⁵, 1.04×10⁵, 1.2×10⁵, 1.4×10⁵, 1.5×10⁵, 2×10⁵, or 3×10⁵ CFUs of a disclosed probiotic or a consortium of disclosed probiotics, at least 1.02×10⁶, 1.04×10⁶, 1.2×10⁶, 1.4×10⁶, 2×10⁶, or 3×10⁶ CFUs of a disclosed probiotic or a consortium of disclosed probiotics, at least 1.02×10⁷, 1.04×10⁷, 1.2×10⁷, 1.4×10⁷, 2×10⁷, or 3×10⁷ CFUs of a disclosed probiotic or a consortium of disclosed probiotics, at least 1.02×10⁸, 1.04×10⁸, 1.2×10⁸, 1.4×10⁸, 2×10⁸, or 3×10⁸ CFUs of a disclosed probiotic or a consortium of disclosed probiotics, at least 1.02×10⁹, 1.04×10⁹, 1.2×10⁹, 1.4×10⁹, 2×10⁹, or 3×10⁹ CFUs of a disclosed probiotic or a consortium of disclosed probiotics, at least 10¹⁰ CFUs of a disclosed probiotic or a consortium of disclosed probiotics, at least 1.02×10¹⁰, 1.04×10¹⁰, 1.2×10¹⁰, 1.4×10¹⁰, 2×10¹⁰, or 3×10¹⁰ CFUs of a disclosed probiotic or a consortium of disclosed probiotics, at least 1.02×10¹¹, 1.04×10¹¹, 1.2×10¹¹, 1.4×10¹¹, 2×10¹¹, or 3×10¹¹ CFUs of a disclosed probiotic or a consortium of disclosed probiotics, or at least 1.02×10¹², 1.04×10¹², 1.2×10¹², 1.4×10¹², 2×10¹², or 3×10¹² CFUs of a disclosed probiotic or a consortium of disclosed probiotics per dose.

In an aspect, a disclosed therapeutically effective dose can comprise any amount or an unlimited amount of bacterial CFUs of the one or more probiotics.

In an aspect of a disclosed pharmaceutical formulation, a disclosed therapeutically effective dose can comprise Corynebacterium sp., Dolosigranulum sp., Streptococcus sp., and Lactobacillus sp. in a ratio of about 1:0.01:0.01:0.01 to about 1:1:1:1. In an aspect of a disclosed pharmaceutical formulation, a disclosed therapeutically effective dose can comprise Corynebacterium sp., Dolosigranulum sp., Streptococcus sp., and Lactobacillus sp. in any ratio.

In an aspect of a disclosed pharmaceutical formulation, a disclosed therapeutically effective dose can comprise a higher dose of Corynebacterium sp., or a higher dose of Dolosigranulum sp., or a higher dose of Streptococcus sp., or a higher dose of Lactobacillus sp. as compared to any other bacteria in the consortium. In an aspect of a disclosed pharmaceutical formulation, a disclosed therapeutically effective dose can comprise a dose of Lactobacillus sp. that is about double that of Corynebacterium sp.

In an aspect, a disclosed pharmaceutical formulation can be prepared for systemic or direct administration. In an aspect, a disclosed pharmaceutical formulation can be prepared for oral administration, intravenous administration, intranasal administration, sublingual administration, intraperitoneal administration, or any combination thereof. In an aspect, a disclosed pharmaceutical formulation can be prepared for any method of administration disclosed herein.

In an aspect, a disclosed pharmaceutical formulation can be prepared for administration via multiple routes either concurrently or sequentially.

In an aspect, a disclosed pharmaceutical formulation can further comprise (i) one or more active agents, (ii) one or more biologically active agents, (iii) one or more pharmaceutically active agents, (iv) one or more immune-based therapeutic agents, (v) one or more clinically approved agents, or (vi) any combination thereof. In an aspect, a disclosed pharmaceutical formulation can comprise one or more anti-bacterial agents. In an aspect of a disclosed pharmaceutical formulation, the dose of an active agent, a biologically active agent, a pharmaceutically active agent, an immune-based therapeutic active agent, a clinically approved agent, or an anti-bacterial agent can be about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed pharmaceutical formulation can be administered to a healthy subject, for example, a subject that does not have an infection and/or is not suspected of having an infection.

In an aspect, a disclosed pharmaceutical formulation can promote a subject's respiratory health, can inhibit and/or prevent the growth and/or spread of pathogenic bacteria and/or S. pneumoniae, can inhibit and/or prevent the adherence and/or colonization of pathogenic bacteria and/or S. pneumoniae, can modulate the endocytosis or paracellular migration of pathogenic bacteria and/or S. pneumoniae, can inhibit and/or prevent the spread of pathogenic bacteria and/or S. pneumoniae by competitive adherence and/or niche occupancy, or any combination thereof.

### 2. Repository of Microbiome Sequencing Data

Disclosed herein is a repository of microbiome sequence data generated by one or more methods disclosed here. Disclosed herein is a repository of microbiome sequence data available at Accession No. PRJNA698366 in the Sequence Read Archive (available at https://www.ncbi.nlm.nih.gov/bioproject/PRJNA698366/). In an aspect, a disclosed repository of microbiome sequence data can comprise data generated from about 1622 biosamples or SRA experiments.

### D. Methods of Promoting Respiratory Health

Disclosed herein is a method of promoting respiratory health in a subject, the method comprising administering to a subject in need thereof a therapeutically effective amount of a biotherapeutic, wherein the biotherapeutic inhibits and/or prevents the growth of one or more pathogenic bacteria in one or more parts of the subject's respiratory system. Disclosed herein is a method of promoting respiratory health in a subject, the method comprising administering to a subject in need thereof a therapeutically effective amount of a biotherapeutic, thereby inhibiting and/or preventing the growth of one or more pathogenic bacteria in one or more parts of the subject's respiratory system.

In an aspect of a disclosed method of promoting respiratory health, a subject can be any subject. In an aspect, a subject that does not have an infection and/or is not suspected of having an infection.

In an aspect, a disclosed method can further comprise characterizing the microbiome of a biological sample. In an aspect, a disclosed microbiome can comprise the nasopharyngeal microbiome, the nasal microbiome, or both.

In an aspect, a disclosed method can further comprise obtaining a biological sample from the subject. In an aspect, a disclosed biological sample can comprise a nasal swab or lavage, a nasopharyngeal swab or lavage, or a pharyngeal swab or lavage, or any combination thereof. In an aspect, a disclosed biological sample can comprise nasal secretion and/or cough droplets.

In an aspect of a disclosed method, characterizing the microbiome can comprise (i) collecting a biological sample from the subject; (ii) extracting nucleic acid from the subject's biological sample; and (iii) sequencing the extracted nucleic acid. In an aspect, sequencing the extracted nucleic acid can generate sequence data. In an aspect, characterizing the microbiome can further comprise analyzing the sequence data using taxonomic classification. In an aspect, using taxonomic classification can comprise PCR amplification. In an aspect, PCR amplification can comprise using primers targeting the 16S rRNA gene. In an aspect, PCR amplification can comprise primers targeting the V4 variable region of the 16S rRNA gene. In an aspect, disclosed primers can comprise a pair of primers. In an aspect, a disclosed pair of primers can comprise a primer having the sequence of SEQ ID NO:01 and a primer having the sequence of SEQ ID NO:02. In an aspect, a disclosed pair of primers can comprise a primer having the sequence of SEQ ID NO:03 and a primer having the sequence of SEQ ID NO:04. In an aspect, a disclosed pair of primers can comprise a primer having the sequence of SEQ ID NO:05 and a primer having the sequence of SEQ ID NO:06.

TABLE 1
Primer Identification and Sequences.

| SEQ | | |
|---|---|---|
| ID NO | Description | Sequence |
| 01 | 515F Parada (FOR barcoded) | GTGYCAGCMGCCGCGGTAA |
| 02 | 806R Apprill (FOR barcoded) | GGACTACNVGGGTWTCTAAT |
| 03 | 515F Caporaso (REV barcoded) | GTGCCAGCMGCCGCGGTAA |
| 04 | 806R Caporaso (REV barcoded) | GGACTACHVGGGTWTCTAAT |
| 05 | 5′ Illumina adapter | AATGATACGGCGACCACCGAGATC |
| | Golay barcode | TACACGCTXXXXXXXXXXXXTATG |
| | Forward primer pad | GTAATTGTGTGYCAGCMGCCGCGG |
| | Forward primer linker | TAA |
| | Forward primer (515F) | |
| 06 | Reverse complement of 3′ Illumina | CAAGCAGAAGACGGCATACGAGAT |
| | adapter | AGTCAGCCAGCCGGACTACNVGGGT |
| | Reverse primer pad | WTCTAAT |
| | Reverse primer linker | |
| | Reverse primer (806R) | |
| 07 | 16S Sequencing Primers-Read 1 | TATGGTAATTGTGTGYCAGCMGCC |
| | Forward primer pad | GCGGTAA |
| | Forward primer linker | |
| | Forward primer | |
| 08 | 16S Sequencing Primers-Read 2 | AGTCAGCCAGCCGGACTACNVGGG |
| | Reverse primer pad | TWTCTAAT |
| | Reverse primer linker | |
| | Reverse primer | |
| 09 | Index sequencing primer | AATGATACGGCGACCACCGAGATC |
| | | TACACGCT |

In an aspect, a disclosed method of promoting respiratory health can further comprise identifying the one or more pathogenic bacteria. In an aspect, the disclosed pathogenic bacteria can comprise Streptococcus sp. In an aspect, Streptococcus sp. can comprise S. pneumoniae. In an aspect, the disclosed pathogenic bacteria can comprise Staphylococcus sp. In an aspect, Staphylococcus sp. can comprise methicillin-sensitive S. aureus or methicillin-resistant S. aureus. In an aspect, the disclosed pathogenic bacteria can comprise Haemophilus influenzae or Moraxella catarrhalis.

In an aspect, a disclosed method of promoting respiratory health can further comprise diagnosing the subject with an infection. In an aspect, a diagnosed infection can comprise a respiratory infection. In an aspect, a diagnosed infection can affect the subject's mouth, nose, ears, sinuses, pharynx, trachea, bronchial tubes, lungs, alveoli, bronchioles, capillaries, lung lobes, pleura, cilia, epiglottis, larynx, or any combination thereof.

In an aspect, a disclosed method of promoting respiratory health can further comprise diagnosing the subject with pneumonia, otitis media, sinusitis, rhinosinusitis, or any combination thereof. In an aspect, otitis media (GM) can be considered a general term for middle-ear inflammation and can be classified clinically as acute otitis media (AOM), recurrent AOM, GM with effusion (OME), or chronic GOVI, chronic suppurative GM (CSGM), or middle ear effusion (MEE).

TABLE 2
Otitis Media Definitions and Terminology

| Preferred Term | Definition | Comment |
|---|---|---|
| Otitis media (OM) | Inflammation of the middle ear | Nonspecific umbrella term for any |
| | without reference to etiology | condition associated with |
| | or pathogenesis | inflammation of the middle ear |
| Acute OM (AOM) | Rapid onset of signs and | Diagnosed when there is moderate-to- |
| | symptoms of inflammation in | severe bulging of the tympanic |
| | the middle ear | membrane; mild bulging of the |
| | | tympanic membrane and recent (<48 |
| | | hours) onset of ear pain or intense |
| | | erythema of the tympanic membrane; |
| | | or acute ear discharge not caused by |
| | | otitis externa (inflammation of the |
| | | external ear canal) |
| Recurrent AOM | Three or more well- | Children without persistent MEE tend |
| | documented and separate | to have a good prognosis and often |
| | AOM episodes in the | improve spontaneously; children with |
| | preceding 6 months or four or | persistent MEE have a poorer |
| | more episodes in the preceding | prognosis and might benefit from |
| | 12 months with more than one | ventilation tubes |
| | episode in the past 6 months | |
| OM with effusion | Fluid in the middle ear without | Diagnosed by one or more of the |
| (OME) | signs or symptoms of acute ear | following: reduced tympanic |
| | infection | membrane mobility on pneumatic |
| | | otoscopy, reduced tympanic |
| | | membrane mobility on |
| | | tympanometry, opaque tympanic |
| | | membrane or a visible air-fluid |
| | | interface behind the tympanic |
| | | membrane on otoscopy |
| Chronic OME | OME persisting for ≥3 months | Chronic OME has much lower rates |
| | from the date of onset (if | of spontaneous resolution than new- |
| | known) or from the date of | onset OME or following an episode of |
| | diagnosis (if onset is unknown) | AOM |
| Chronic | Chronic inflammation of the | No consensus on the duration of ear |
| suppurative OM | middle ear and mastoid | discharge needed for diagnosis, with |
| (CSOM) | mucosa with a non-intact | recommendations ranging from 2 |
| | tympanic membrane | weeks to at least 3 months |
| | (perforation or ventilation | |
| | tube) and persistent ear | |
| | discharge | |
| Middle ear effusion | Fluid in the middle ear from | MEE is present with both OME and |
| (MEE) | any cause | AOM and might persist for weeks or |
| | | months after the signs and symptoms |
| | | of AOM resolve |

In an aspect, sinusitis can occur when the tissue lining of the sinuses becomes swollen or inflamed. Sinusitis can comprise acute sinusitis (e.g., when symptoms are present for 4 weeks or less), chronic sinusitis (e.g., when the swelling of the sinuses is present for longer than 3 months), or subacute sinusitis (e.g., when the swelling is present between one and three months). In an aspect, rhinosinusitis can occur when the mucosal lining in the paranasal sinuses and nasal cavity becomes inflamed. Most cases of acute rhinosinusitis are caused by viral upper respiratory infections.

In an aspect, a disclosed biotherapeutic can comprise a probiotic, a consortium of probiotics, factors secreted from a probiotic, factors secreted from a consortium of probiotics, or any combination thereof.

In an aspect, a disclosed biotherapeutic can comprise pharmaceutical formulation comprising a probiotic, a consortium of probiotics, factors secreted from a probiotic, factors secreted from a consortium of probiotics, or any combination thereof, and a pharmaceutically acceptable carrier and/or excipient. In an aspect, a disclosed pharmaceutical formulation can comprise at least one lyoprotectant. A disclosed lyoprotectant can comprise peptone, glycerol, lactose, gelatin, glucose, sucrose, trehalose, dextran, maltodextrin, adonitol, sodium glutamate, or any combination thereof. In an aspect, a disclosed biotherapeutic can be lyophilized or freeze-dried such that, for example, a disclosed probiotic, a disclosed consortium of probiotics, disclosed factors secreted from a probiotic, disclosed factors secreted from a consortium of probiotics, or any combination thereof are all lyophilized or freeze-dried.

In an aspect, a disclosed consortium of probiotics can comprise at least one strain from the bacterial genus Corynebacterium, at least one strain from the bacterial genus Dolosigranulum, at least one strain from the bacterial genus Streptococcus, and at least one strain from the bacterial genus Lactobacillus. In an aspect of a disclosed method of promoting respiratory health, a disclosed biotherapeutic can comprises one or more probiotics.

In an aspect, one or more disclosed probiotics can comprise at least one strain from the bacterial genus one Corynebacterium sp. In an aspect, one or more disclosed probiotics can comprise at least one strain from the bacterial genus Corynebacterium and at least one strain from the bacterial genus Dolosigranulum. In an aspect, one or more disclosed probiotics can comprise at least one strain from the bacterial genus Corynebacterium, at least one strain from the bacterial genus Dolosigranulum, and at least one strain from the bacterial genus Streptococcus. In an aspect, one or more disclosed probiotics can comprise at least one strain from the bacterial genus Corynebacterium, at least one strain from the bacterial genus Dolosigranulum, and at least one strain from the bacterial genus Lactobacillus. In an aspect, one or more disclosed probiotics can comprise at least one strain from the bacterial genus Corynebacterium and at least one at least one strain from the bacterial genus Streptococcus. In an aspect, one or more disclosed probiotics can comprise at least one strain from the bacterial genus Corynebacterium, at least one strain from the bacterial genus Streptococcus, and at least one strain from the bacterial genus Lactobacillus. In an aspect, one or more probiotics can comprise at least one strain from the bacterial genus Corynebacterium and at least one strain from the bacterial genus Lactobacillus. In an aspect, one or more probiotics can comprise at least one strain from the bacterial genus Corynebacterium, at least one strain from the bacterial genus Dolosigranulum, at least one strain from the bacterial genus Streptococcus, and at least one strain from the bacterial genus Lactobacillus.

In an aspect of a disclosed method of promoting respiratory health, a disclosed biotherapeutic can comprise one or more factors secreted by a disclosed probiotic or secreted by a disclosed consortium of probiotics. In an aspect of a disclosed pharmaceutical formulation, a disclosed biotherapeutic can comprise one or more factors secreted by at least one strain from the bacterial genus Corynebacterium, at least one strain from the bacterial genus Dolosigranulum, at least one strain from the bacterial genus Streptococcus, and at least one strain from the bacterial genus Lactobacillus. In an aspect of a disclosed pharmaceutical formulation, a disclosed biotherapeutic can comprise one or more factors secreted by at least one strain from the bacterial genus Corynebacterium. In an aspect of a disclosed pharmaceutical formulation, a disclosed biotherapeutic can comprise one or more factors secreted by at least one strain from the bacterial genus Corynebacterium and at least one strain from the bacterial genus Dolosigranulum. In an aspect of a disclosed pharmaceutical formulation, a disclosed biotherapeutic can comprise one or more factors secreted by at least one strain from the bacterial genus Corynebacterium, at least one strain from the bacterial genus Dolosigranulum, and at least one train from the bacterial genus Streptococcus. In an aspect of a disclosed pharmaceutical formulation, a disclosed biotherapeutic can comprise one or more factors secreted by at least one strain from the bacterial genus Corynebacterium, at least one strain from the bacterial genus Dolosigranulum, and at least one strain from the bacterial genus Lactobacillus. In an aspect of a disclosed pharmaceutical formulation, a disclosed biotherapeutic can comprise one or more factors secreted by at least one strain from the bacterial genus Corynebacterium and at least one strain from the bacterial genus Streptococcus. In an aspect of a disclosed pharmaceutical formulation, a disclosed biotherapeutic can comprise comprises one or more factors secreted by at least one strain from the bacterial genus Corynebacterium, at least one strain from the bacterial genus Streptococcus, and at least one strain from the bacterial genus Lactobacillus. In an aspect of a disclosed pharmaceutical formulation, a disclosed biotherapeutic can comprise one or more factors secreted by at least one strain from the bacterial genus Corynebacterium and at least one strain from the bacterial genus Lactobacillus.

In an aspect of a disclosed pharmaceutical formulation, a disclosed strain from the bacterial genus Corynebacterium can comprise C. accolens, C. afermentans subsp. afermentans, C. afermentans subsp. lipophilum, C. ammoniagenes, C. amycolatum, C. appendicis, C. aquaticum, C. argentoratense, C. atypicum, C. aurimucosum, C. auris, C. bovis, C. canis, C. confusum, C. coyleae, C. diphtheriae, C. durum, C. efficiens, C. equi (now Rhodococcus equi), C. falsenii, C. flavescens, C. freiburgense, C. freneyi, C. glucuronolyticum, C. glutamicum, C. granulosum, C. haemolyticum, C. halofytica, C. kroppenstedtii, C. hansenii, C. imitans, C. jeikeium (group JK), C. kroppenstedtii, C. kutscheri, C. lipophiloflavum, C. macginleyi, C. massiliense, C. mastitidis-like, C. matruchotii, C. minutissimum, C. mucifaciens, C. mycetoides, C. ovis, C. parvum (Propionibacterium acnes), C. paurometabolum, C. pilbarense, C. propinquum, C. pseudodiphtheriticum (C. hofmannii), C. pseudotuberculosis, C. pyogenes-Trueperella pyogenes, C. pyruviciproducens, C. renale, C. resistans, C. riegelii, C. simulans, C. singular, C. spec, C. sputi, C. stationis, C. striatum, C. sundsvallense, C. tenuis, C. thomsenii, C. timonense, C. tuberculostearicum, C. tuscaniense, C. ulcerans, C. urealyticum (group D2), C. urealyticum, C. xerosis, Corynebacterium_BWA136, Corynebacterium_BWA297, Corynebacterium_DU041, Corynebacterium_DU044, or any combination thereof. In an aspect of a disclosed pharmaceutical formulation, a disclosed strain from the bacterial genus Corynebacterium can comprise C. accolens, C. coyleae, C. propinquum, C. pseudodiphtheriticum, C. amycolatum, C. glutamicum, C. aurimucosum, C. tuberculostearicum, C. afermentans, C. striatum, Corynebacterium_DU041, Corynebacterium_DU044, Corynebacterium_BWA136, Corynebacterium_BWA297, or any combination thereof. In an aspect of a disclosed pharmaceutical formulation, a disclosed strain from the bacterial genus Corynebacterium can comprise any species identified in Table 13 (supra).

In an aspect of a disclosed method of promoting respiratory health, a disclosed therapeutically effective dose can comprise a total of at least 100 bacterial CFUs of the one or more probiotics, a total of at least 200 to at least 300 bacterial CFUs of the one or more probiotics, a total of at least 300 to at least 400 bacterial CFUs of the one or more probiotics, a total of at least 400 to at least 500 bacterial CFUs of the one or more probiotics, a total of at least 500 to at least 600 bacterial CFUs of the one or more probiotics, a total of at least 600 to at least 700 bacterial CFUs of the one or more probiotics, a total of at least 700 to at least 800 bacterial CFUs of the one or more probiotics, a total of at least 800 to at least 900 bacterial CFUs to at least 1000 bacterial CFUs of the one or more probiotics, or a total of at least 1000 bacterial CFUs of one or more disclosed probiotics (such as, for example, at least one strain from the bacterial genus Corynebacterium, at least one strain from the bacterial genus Dolosigranulum, at least one strain from the bacterial genus Streptococcus, and at least one strain from the bacterial genus Lactobacillus, or any combination thereof).

In an aspect of a disclosed method of promoting respiratory health, a disclosed therapeutically effective dose can comprise a total of at least 1000 to at least 2000 bacterial CFUs of the one or more probiotics, a total of at least 2000 to at least 3000 bacterial CFUs of the one or more probiotics, a total of at least 3000 to at least 4000 bacterial CFUs of the one or more probiotics, a total of at least 4000 to at least 5000 bacterial CFUs of the one or more probiotics, a total of at least 5000 to at least 6000 bacterial CFUs of the one or more probiotics, a total of at least 6000 to at least 7000 bacterial CFUs of the one or more probiotics, a total of at least 8000 to at least 9000 bacterial CFUs of the one or more probiotics, or a total of at least 10,000 bacterial CFUs of one or more disclosed probiotics (such as, for example, at least one strain from the bacterial genus Corynebacterium, at least one strain from the bacterial genus Dolosigranulum, at least one strain from the bacterial genus Streptococcus, and at least one strain from the bacterial genus Lactobacillus, or any combination thereof).

In an aspect of a disclosed method of promoting respiratory health, a disclosed therapeutically effective dose can comprise a total of at least 100 bacterial CFUs of Corynebacterium sp., Dolosigranulum sp., Streptococcus sp., Lactobacillus sp., or any combination thereof, a total of at least 200 to at least 300 bacterial CFUs of Corynebacterium sp., Dolosigranulum sp., Streptococcus sp., Lactobacillus sp., or any combination thereof, a total of at least 300 to at least 400 bacterial CFUs of Corynebacterium sp., Dolosigranulum sp., Streptococcus sp., Lactobacillus sp., or any combination thereof, a total of at least 400 to at least 500 bacterial CFUs of Corynebacterium sp., Dolosigranulum sp., Streptococcus sp., Lactobacillus sp., or any combination thereof, a total of at least 500 to at least 600 bacterial CFUs of Corynebacterium sp., Dolosigranulum sp., Streptococcus sp., Lactobacillus sp., or any combination thereof, a total of at least 600 to at least 700 bacterial CFUs of Corynebacterium sp., Dolosigranulum sp., Streptococcus sp., Lactobacillus sp., or any combination thereof, a total of at least 700 to at least 800 bacterial CFUs of Corynebacterium sp., Dolosigranulum sp., Streptococcus sp., Lactobacillus sp., or any combination thereof, a total of at least 800 to at least 900 bacterial CFUs of Corynebacterium sp., Dolosigranulum sp., Streptococcus sp., Lactobacillus sp., or any combination thereof, a total of at least 900 to at least 1000 bacterial CFUs of Corynebacterium sp., Dolosigranulum sp., Streptococcus sp., Lactobacillus sp., or any combination thereof, or a total of at least 1000 bacterial CFUs of Corynebacterium sp., Dolosigranulum sp., Streptococcus sp., Lactobacillus sp., or any combination thereof.

In an aspect of a disclosed method of promoting respiratory health, a disclosed therapeutically effective dose can comprise a total of at least 1000 to at least 2000 bacterial CFUs of Corynebacterium sp., Dolosigranulum sp., Streptococcus sp., Lactobacillus sp., or any combination thereof, a total of at least 2000 to at least 3000 bacterial CFUs of Corynebacterium sp., Dolosigranulum sp., Streptococcus sp., Lactobacillus sp., or any combination thereof, a total of at least 3000 to at least 4000 bacterial CFUs of Corynebacterium sp., Dolosigranulum sp., Streptococcus sp., Lactobacillus sp., or any combination thereof, a total of at least 4000 to at least 5000 bacterial CFUs of the Corynebacterium sp., Dolosigranulum sp., Streptococcus sp., Lactobacillus sp., or any combination thereof, a total of at least 5000 to at least 6000 bacterial CFUs of Corynebacterium sp., Dolosigranulum sp., Streptococcus sp., Lactobacillus sp., or any combination thereof, a total of at least 6000 to at least 7000 bacterial CFUs of Corynebacterium sp., Dolosigranulum sp., Streptococcus sp., Lactobacillus sp., or any combination thereof, a total of at least 8000 to at least 9000 bacterial CFUs of Corynebacterium sp., Dolosigranulum sp., Streptococcus sp., Lactobacillus sp., or any combination thereof, or a total of at least 10,000 bacterial CFUs of Corynebacterium sp., Dolosigranulum sp., Streptococcus sp., Lactobacillus sp., or any combination thereof.

In an aspect, a disclosed therapeutically effective dose can comprise a total of at least 10⁴ to at least 10⁵ bacterial CFUs of the one or more probiotics, a total of at least 10⁵ to at least 10⁶ bacterial CFUs of the one or more probiotics, a total of at least 10⁶ to at least 10⁷ bacterial CFUs of the one or more probiotics, a total of at least 10⁷ to at least 10⁸ bacterial CFUs of the one or more probiotics, a total of at least 10⁸ to at least 10⁹ bacterial CFUs of the one or more probiotics, a total of at least 10⁹ to at least 10¹⁰ bacterial CFUs of the one or more probiotics, a total of at least 10¹⁰ to at least 10¹¹ bacterial CFUs of the one or more probiotics, a total of at least 10¹¹ to at least 10¹² bacterial CFUs of the one or more probiotics, a total of at least 10¹² bacterial CFUs of the one or more probiotics, or more than 10¹² bacterial CFUs of one or more disclosed probiotics (such as, for example, at least one strain from the bacterial genus Corynebacterium, at least one strain from the bacterial genus Dolosigranulum, at least one strain from the bacterial genus Streptococcus, and at least one strain from the bacterial genus Lactobacillus, or any combination thereof).

In an aspect, a disclosed therapeutically effective dose can comprise at least 10³, 10⁴, 10⁵, 10⁶, 10⁷, 10⁸, 10⁹, 10¹⁰, 10¹¹, or 10¹² CFUs of a disclosed probiotic or a consortium of disclosed probiotics, preferably at least 1.2×10³, 1.4×10³, 2×10³, or 3×10³ CFUs of a disclosed probiotic or a consortium of disclosed probiotics, at least 5×10³, 5.02×10³, 5.04×10³, 5.2×10³, or 5.4×10³ CFUs of a disclosed probiotic or a consortium of disclosed probiotics, at least 1.02×10⁴, 1.04×10⁴, 1.2×10⁴, 1.4×10⁴, 1.5×10⁴, 2×10⁴, or 3×10⁴ CFUs of a disclosed probiotic or a consortium of disclosed probiotics, at least 5×10⁴, 5.02×10⁴, 5.04×10⁴, 5.2×10⁴, or 5.4×10⁴ CFUs of a disclosed probiotic or a consortium of disclosed probiotics, at least 1.02×10⁵, 1.04×10⁵, 1.2×10⁵, 1.4×10⁵, 1.5×10⁵, 2×10⁵, or 3×10⁵ CFUs of a disclosed probiotic or a consortium of disclosed probiotics, at least 1.02×10⁶, 1.04×10⁶, 1.2×10⁶, 1.4×10⁶, 2×10⁶, or 3×10⁶ CFUs of a disclosed probiotic or a consortium of disclosed probiotics, at least 1.02×10⁷, 1.04×10⁷, 1.2×10⁷, 1.4×10⁷, 2×10⁷, or 3×10⁷ CFUs of a disclosed probiotic or a consortium of disclosed probiotics, at least 1.02×10⁸, 1.04×10⁸, 1.2×10⁸, 1.4×10⁸, 2×10⁸, or 3×10⁸ CFUs of a disclosed probiotic or a consortium of disclosed probiotics, at least 1.02×10⁹, 1.04×10⁹, 1.2×10⁹, 1.4×10⁹, 2×10⁹, or 3×10⁹ CFUs of a disclosed probiotic or a consortium of disclosed probiotics, at least 10¹⁰ CFUs of a disclosed probiotic or a consortium of disclosed probiotics, at least 1.02×10¹⁰, 1.04×10¹⁰, 1.2×10¹⁰, 1.4×10¹⁰, 2×10¹⁰, or 3×10¹⁰ CFUs of a disclosed probiotic or a consortium of disclosed probiotics, at least 1.02×10¹¹, 1.04×10¹¹, 1.2×10¹¹, 1.4×10¹¹, 2×10¹¹, or 3×10¹¹ CFUs of a disclosed probiotic or a consortium of disclosed probiotics, or at least 1.02×10¹², 1.04×10¹², 1.2×10¹², 1.4×10¹², 2×10¹², or 3×10¹² CFUs of a disclosed probiotic or a consortium of disclosed probiotics per dose.

In an aspect, a disclosed therapeutically effective dose can comprise any amount or an unlimited amount of bacterial CFUs of the one or more probiotics.

In an aspect of a disclosed method of promoting respiratory health, a disclosed therapeutically effective dose can comprise Corynebacterium sp., Dolosigranulum sp., Streptococcus sp., and Lactobacillus sp. in a ratio of about 1:0.01:0.01:0.01 to about 1:1:1:1. In an aspect, a disclosed therapeutically effective dose can comprise Corynebacterium sp., Dolosigranulum sp., Streptococcus sp., and Lactobacillus sp. in any ratio. In an aspect of a disclosed pharmaceutical formulation, a disclosed therapeutically effective dose can comprise a higher dose of Corynebacterium sp., or a higher dose of Dolosigranulum sp., or a higher dose of Streptococcus sp., or a higher dose of Lactobacillus sp. as compared to any other bacteria in the consortium. In an aspect, a disclosed therapeutically effective dose can comprise a dose of Lactobacillus sp. that is about double that of Corynebacterium sp.

In an aspect, a disclosed method of promoting respiratory health can further comprise treating the subject. In an aspect, treating the subject can comprise treating the subject's respiratory infection. In an aspect, treating the subject can comprise treating the subject's non-respiratory infection.

In an aspect, a disclosed method of promoting respiratory health can comprise further comprising administering to the subject a therapeutically effective amount of one or more anti-bacterial agents. Anti-bacterial agents and combinations of anti-bacterial agents are known to the art and discussed supra in Part VII(C)(1).

In an aspect, a disclosed method of promoting respiratory health can further comprise administering to the subject a therapeutically effective amount of (i) one or more active agents, (ii) one or more biologically active agents, (iii) one or more pharmaceutically active agents, (iv) one or more immune-based therapeutic agents, (v) one or more clinically approved agents, or (vi) any combination thereof.

In an aspect of a disclosed pharmaceutical formulation, the dose of an active agent, a biologically active agent, a pharmaceutically active agent, an immune-based therapeutic active agent, a clinically approved agent, or an anti-bacterial agent can be about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, administering a disclosed agent can comprise systemic or direct administration. In an aspect, administering a disclosed anti-bacterial agent can comprise oral administration, intravenous administration, intranasal administration, intraperitoneal administration, or any combination thereof. In an aspect, administering a disclosed agent can be administered by any method of administration disclosed herein. In an aspect, a disclosed agent can be administered via multiple routes either concurrently or sequentially. A skilled clinician can determine the best route of administration for a disclosed agent to a subject at a given time.

In an aspect, a disclosed agent can inherently encompass a pharmaceutically acceptable salt thereof.

In an aspect, a disclosed method of promoting respiratory health can further comprise increasing the relative abundance of one or more probiotics in the subject's microbiome. In an aspect, a disclosed method of promoting respiratory health can further comprise increasing the relative abundance of one or more of Corynebacterium sp., Dolosigranulum sp., Streptococcus sp., and Lactobacillus sp. in the subject's microbiome. In an aspect, a disclosed method of promoting respiratory health can further comprise increasing the relative abundance of one or more of at least one strain from the bacterial genus Corynebacterium, at least one strain from the bacterial genus Dolosigranulum, at least one strain from the bacterial genus Streptococcus, and at least one strain from the bacterial genus Lactobacillus, or any combination thereof in the subject's microbiome. In an aspect, a disclosed method of promoting respiratory health can further comprise increasing the relative abundance of at least one strain from the bacterial genus Corynebacterium in the subject's microbiome. In an aspect, a disclosed method of promoting respiratory health can further comprise increasing the relative abundance of at least one strain from the bacterial genus Dolosigranulum in the subject's microbiome. In an aspect, a disclosed method of promoting respiratory health can further comprise increasing the relative abundance of at least one strain from the bacterial genus Streptococcus in the subject's microbiome. In an aspect, a disclosed method of promoting respiratory health can further comprise increasing the relative abundance of at least one strain from the bacterial genus Lactobacillus in the subject's microbiome.

In an aspect, a disclosed method of promoting respiratory health can further comprise decreasing the relative abundance of one or more pathogenic bacteria in the subject's microbiome. In an aspect, a disclosed method of promoting respiratory health can further comprise decreasing the relative abundance of S. pneumoniae in the subject's microbiome. In an aspect, a disclosed method of promoting respiratory health can further comprise decreasing the relative abundance of S. aureus in the subject's microbiome. In an aspect, a disclosed method of promoting respiratory health can further comprise decreasing the relative abundance of Haemophilus influenzae and/or Moraxella catarrhalis in the subject's microbiome.

In an aspect, a disclosed method of promoting respiratory health can further comprise administering to the subject one or more therapeutic agents. In an aspect, a disclosed therapeutic agent can comprise a biologically active agent, a pharmaceutically active agent, an anti-bacterial agent, an anti-fungal agent, an anti-viral agent, a corticosteroid, an analgesic, an immunostimulant, an immune-based product, or any combination thereof. In an aspect of a disclosed method of promoting respiratory health, administering can comprise intranasal administration, oral administration, sublingual administration, or any combination thereof.

In an aspect of a disclosed method of promoting respiratory health, a subject can be a healthy subject, for example, a subject that does not have an infection and/or is not suspected of having an infection.

In an aspect, a subject can be an adult, a child, or an infant. In an aspect, a subject can be a neonate. In an aspect, a subject can be a premature infant. In an aspect, a subject can be immune-compromised. In an aspect, a subject can have diabetes or a chronic disease (e.g., heart disease, kidney disease, or liver disease). In an aspect, a subject can have HIV. In an aspect, a subject can have cancer or has had cancer. In an aspect, a subject can be the recipient of one or more solid organ transplants. In an aspect, a subject can have nephrotic syndrome. In an aspect, a subject can be alcoholic or can smoke cigarettes. In an aspect, a subject can have a chronic lung disease. In an aspect, a disclosed chronic lung disease can comprise emphysema, chronic obstructive lung disease, chronic obstructive pulmonary disease, asthma, or any combination thereof.

In an aspect, a subject can be diagnosed with an infection of one or more parts of the respiratory system. In an aspect, a subject can be suspected of having an infection in one or more parts of the respiratory system. In an aspect, a disclosed therapeutically effective amount of a biotherapeutic can comprise an amount sufficient to inhibit and/or prevent the growth of one or more pathogenic bacteria in one or more parts of the subject's respiratory system. In an aspect, a disclosed therapeutically effective amount of a biotherapeutic can comprise an amount sufficient to inhibit and/or prevent the spread of one or more pathogenic bacteria from a first part of the subject's respiratory system to a second part of the subject's respiratory system.

In an aspect, one or more disclosed probiotics can inhibit and/or prevent the growth and/or spread of one or more pathogenic bacteria using competitive adherence and/or non-adherence mechanisms (e.g., secretion of antimicrobial factors). In an aspect, one or more disclosed probiotics can inhibit and/or prevent the adherence and/or colonization of one or more pathogenic bacteria. In an aspect, one or more disclosed probiotics can modulate the endocytosis or paracellular migration of one or more pathogenic bacteria. In an aspect, one or more disclosed probiotics can inhibit and/or prevent the spread of one or more pathogenic bacteria by competitive adherence and/or niche occupancy.

In an aspect, one or more disclosed probiotics can inhibit and/or prevent the growth and/or spread of S. pneumoniae using competitive adherence and/or non-adherence mechanisms (e.g., secretion of antimicrobial factors). In an aspect, one or more disclosed probiotics can inhibit and/or prevent the adherence and/or colonization of S. pneumoniae. In an aspect, one or more disclosed probiotics can modulate the endocytosis or paracellular migration of S. pneumoniae. In an aspect, one or more disclosed probiotics can inhibit and/or prevent the spread S. pneumoniae by competitive adherence and/or niche occupancy.

In an aspect, one or more disclosed probiotics can secrete one or more factors. In an aspect, a disclosed secreted factor can be species-dependent and/or strain-dependent. In an aspect, a disclosed secreted factor can inhibit and/or prevent the growth and/or spread of one or more pathogenic bacteria. In an aspect, a disclosed secreted factor can inhibit and/or prevent the adherence and/or colonization of one or more pathogenic bacteria. In an aspect, a disclosed secreted factor can modulate the endocytosis or paracellular migration of one or more pathogenic bacteria. In an aspect, a disclosed secreted factor can inhibit and/or prevent the spread of one or more pathogenic bacteria by competitive adherence and/or niche occupancy.

In an aspect, a disclosed secreted factor can inhibit and/or prevent the growth and/or spread of S. pneumoniae. In an aspect, a disclosed secreted factor can inhibit and/or prevent the adherence and/or colonization of S. pneumoniae. In an aspect, a disclosed secreted factor can modulate the endocytosis or paracellular migration of S. pneumoniae. In an aspect, a disclosed secreted factor can inhibit and/or prevent the spread of S. pneumoniae by competitive adherence and/or niche occupancy.

In an aspect of a disclosed method of promoting respiratory health, Corynebacterium sp., Dolosigranulum sp., Streptococcus sp., Lactobacillus sp., or any combination thereof can secrete one or more factors that inhibit and/or prevent the growth and/or spread of one or more pathogenic bacteria. In an aspect of a disclosed method of promoting respiratory health, Corynebacterium sp., Dolosigranulum sp., Streptococcus sp., Lactobacillus sp., or any combination thereof can secrete one or more factors that inhibit and/or prevent the adherence and/or colonization of one or more pathogenic bacteria. In an aspect of a disclosed method of promoting respiratory health, Corynebacterium sp., Dolosigranulum sp., Streptococcus sp., Lactobacillus sp., or any combination thereof can secrete one or more secreted factors that modulate the endocytosis or paracellular migration of one or more pathogenic bacteria. In an aspect of a disclosed method of promoting respiratory health, Corynebacterium sp., Dolosigranulum sp., Streptococcus sp., Lactobacillus sp., or any combination thereof can secrete one or more secreted factors inhibit and/or prevent the spread of one or more pathogenic bacteria by competitive adherence and/or niche occupancy. In an aspect of a disclosed method of promoting respiratory health, Corynebacterium sp., Dolosigranulum sp., Streptococcus sp., Lactobacillus sp., or any combination thereof can secrete one or more secreted factors inhibit and/or prevent the growth and/or spread of S. pneumoniae. In an aspect of a disclosed method of promoting respiratory health, Corynebacterium sp., Dolosigranulum sp., Streptococcus sp., Lactobacillus sp., or any combination thereof can secrete one or more factors inhibit and/or prevent the adherence and/or colonization of S. pneumoniae. In an aspect of a disclosed method of promoting respiratory health, Corynebacterium sp., Dolosigranulum sp., Streptococcus sp., Lactobacillus sp., or any combination thereof secrete one or more factors can modulate the endocytosis or paracellular migration of S. pneumoniae. In an aspect of a disclosed method of promoting respiratory health, Corynebacterium sp., Dolosigranulum sp., Streptococcus sp., Lactobacillus sp., or any combination thereof can secrete one or more factors inhibit and/or prevent the spread of S. pneumoniae by competitive adherence and/or niche occupancy.

In an aspect of a disclosed method of promoting respiratory health, Corynebacterium sp. can secrete one or more factors that inhibit and/or prevent the growth and/or spread of one or more pathogenic bacteria. In an aspect of a disclosed method of promoting respiratory health, Corynebacterium sp. can secrete one or more factors that inhibit and/or prevent the adherence and/or colonization of one or more pathogenic bacteria. In an aspect of a disclosed method of promoting respiratory health, Corynebacterium sp. can secrete one or more secreted factors that modulate the endocytosis or paracellular migration of one or more pathogenic bacteria. In an aspect of a disclosed method of promoting respiratory health, Corynebacterium sp. can secrete one or more secreted factors that inhibit and/or prevent the spread of one or more pathogenic bacteria by competitive adherence and/or niche occupancy.

In an aspect of a disclosed method of promoting respiratory health, Corynebacterium sp. can secrete one or more secreted factors that inhibit and/or prevent the growth and/or spread of S. pneumoniae. In an aspect of a disclosed method of promoting respiratory health, Corynebacterium sp. can secrete one or more factors that inhibit and/or prevent the adherence and/or colonization of S. pneumoniae. In an aspect of a disclosed method of promoting respiratory health, Corynebacterium sp. can secrete one or more factors that modulate the endocytosis or paracellular migration of S. pneumoniae. In an aspect of a disclosed method of promoting respiratory health, Corynebacterium sp. can secrete one or more factors that inhibit and/or prevent the spread of S. pneumoniae by competitive adherence and/or niche occupancy.

In an aspect, a disclosed method of promoting respiratory health can comprise a 10%, 20%, 30%, 40%, 50%, 60%, 70%, 80%, 90%, 100%, or any amount of inhibiting and/or preventing the growth and/or spread of one or more pathogenic bacteria (e.g., S. pneumoniae) when compared to a control subject (such as, for example, a subject that has not received a disclosed biotherapeutic). In an aspect, a disclosed method of promoting respiratory health can comprise a 10-20%, 20-30%, 30-40%, 40-50%, 50-60%, 60-70%, 70-80%, 80-90%, or 90-100% or any amount of inhibiting and/or preventing the growth and/or spread of one or more pathogenic bacteria (e.g., S. pneumoniae) when compared to a control subject (such as, for example, a subject that has not received a disclosed biotherapeutic).

In an aspect, a disclosed method of promoting respiratory health can comprise a 10%, 20%, 30%, 40%, 50%, 60%, 70%, 80%, 90%, 100%, or any amount of inhibiting and/or preventing the adherence and/or colonization of one or more pathogenic bacteria (e.g., S. pneumoniae) when compared to a control subject (such as, for example, a subject that has not received a disclosed biotherapeutic). In an aspect, a disclosed method of promoting respiratory health can comprise a 10-20%, 20-30%, 30-40%, 40-50%, 50-60%, 60-70%, 70-80%, 80-90%, or 90-100% or any amount of inhibiting and/or preventing the adherence and/or colonization of one or more pathogenic bacteria (e.g., S. pneumoniae) when compared to a control subject (such as, for example, a subject that has not received a disclosed biotherapeutic).

In an aspect, a disclosed method of promoting respiratory health can comprise a 10%, 20%, 30%, 40%, 50%, 60%, 70%, 80%, 90%, 100%, or any amount of modulating the endocytosis or paracellular migration of one or more pathogenic bacteria (e.g., S. pneumoniae) when compared to a control subject (such as, for example, a subject that has not received a disclosed biotherapeutic). In an aspect, a disclosed method of promoting respiratory health can comprise a 10-20%, 20-30%, 30-40%, 40-50%, 50-60%, 60-70%, 70-80%, 80-90%, or 90-100% or any amount of modulating the endocytosis or paracellular migration of one or more pathogenic bacteria (e.g., S. pneumoniae) when compared to a control subject (such as, for example, a subject that has not received a disclosed biotherapeutic).

In an aspect, a disclosed method of promoting respiratory health can comprise a 10%, 20%, 30%, 40%, 50%, 60%, 70%, 80%, 90%, 100%, or any amount of inhibiting and/or preventing the spread of one or more pathogenic bacteria (e.g., S. pneumoniae) by competitive adherence and/or niche occupancy when compared to a control subject (such as, for example, a subject that has not received a disclosed biotherapeutic). In an aspect, a disclosed method of promoting respiratory health can comprise a 10-20%, 20-30%, 30-40%, 40-50%, 50-60%, 60-70%, 70-80%, 80-90%, or 90-100% or any amount of inhibiting and/or preventing the spread of one or more pathogenic bacteria (e.g., S. pneumoniae) by competitive adherence and/or niche occupancy when compared to a control subject (such as, for example, a subject that has not received a disclosed biotherapeutic).

In an aspect, a disclosed method of promoting respiratory health can comprise repeating the administering of one or more disclosed biotherapeutics, one or more disclosed secreted factors, a disclosed consortium of probiotics, one or more disclosed probiotics, or any combination thereof. For example, in an aspect, repeating the administering can comprise administering one or more times daily (e.g., 1, 2, 3, or 4 times). In an aspect, repeating the administering can comprise administering on several consecutive days (e.g., for 1, 2, 3, 4, 5, 6, 7, 8, 9, or 10 days, or more than 10 days), weeks (e.g., for 1, 2, 3, 4, 5, 6, 7, 8, 9, or 10 weeks, or more than 10 weeks), or months (e.g., for 1, 2, 3, 4, 5, or 6 months, or more than 6 months). In an aspect, a disclosed biotherapeutic, a disclosed secreted factor, a disclosed consortium of probiotics, a disclosed probiotic, or any combination thereof can be administered 1-3 times per day for about 7 to 21 days.

In an aspect, a disclosed method of promoting respiratory health can comprise monitoring the subject. In an aspect, monitoring the subject can comprise monitoring the subject for the development of adverse effects. In an aspect, in the absence of adverse effects, a disclosed method can comprise continuing to treat the subject. In an aspect, continuing to treat the subject can comprise continuing to administer to the subject one or more disclosed biotherapeutics, one or more disclosed secreted factors, a disclosed consortium of probiotics, one or more disclosed probiotics, or any combination thereof. In an aspect, in the presence of adverse effects, a disclosed method can comprise modifying one or more steps of the method. In an aspect, modifying one or more steps of a disclosed method can comprise modifying the administering step. In an aspect, modifying the administering step can comprise changing the amount of one or more disclosed biotherapeutics, one or more disclosed secreted factors, a disclosed consortium of probiotics, one or more disclosed probiotics, or any combination thereof administered to the subject, changing the frequency of administration of one or more disclosed biotherapeutics, one or more disclosed secreted factors, a disclosed consortium of probiotics, one or more disclosed probiotics, or any combination thereof, changing the duration of administration of one or more disclosed biotherapeutics, one or more disclosed secreted factors, a disclosed consortium of probiotics, one or more disclosed probiotics, or any combination thereof, changing the route of administration of one or more disclosed biotherapeutics, one or more disclosed secreted factors, a disclosed consortium of probiotics, one or more disclosed probiotics, or any combination thereof, or any combination of disclosed changes.

In an aspect, a disclosed method of promoting respiratory health can comprise manipulating the microbiome to inhibit and/or prevent infection caused by one or more pathogenic bacteria, such as an infection caused by S. pneumoniae. In an aspect, a disclosed method of promoting respiratory health can comprise manipulating the microbiome to decrease and/or minimize the risk of infection caused by one or more pathogenic bacteria, such as an infection caused by S. pneumoniae. For example, in an aspect, when the relative abundance of one or more disclosed Corynebacterium sp. is below a threshold level of 1.2×10⁶ 16S rRNA gene copies per biological sample, then the risk of colonization by S. pneumoniae is increased and/or higher and/or enhanced. In an aspect, when the relative abundance of Corynebacterium sp. is below a threshold level of 1.2×10⁶ 16S rRNA gene copies per biological sample, then a disclosed method can further comprise administering and/or continuing to administer a therapeutically effective amount of a disclosed biotherapeutic. In an aspect, when the relative abundance of Corynebacterium sp. is above 1.2×10⁶ 16S rRNA gene copies per biological sample, then a disclosed method can further comprise continuing to monitor the subject.

For example, in an aspect, when the relative abundance of one or more disclosed Corynebacterium sp. is less than about 50%, about 60%, about 70%, about 80%, or 90% of the microbiome, then the risk of colonization by S. pneumoniae is increased and/or higher and/or enhanced. For example, in an aspect, when the relative abundance of one or more disclosed Corynebacterium sp. is about 50% to about 60%, about 60% to about 70%, about 70% to about 80%, about 80% to about 90% of the microbiome, then the risk of colonization by S. pneumoniae is increased and/or higher and/or enhanced. In an aspect, when the relative abundance of Corynebacterium sp. is about 50% to about 60%, about 60% to about 70%, about 70% to about 80%, about 80% to about 90% of the microbiome, then a disclosed method can further comprise administering and/or continuing to administer a therapeutically effective amount of a disclosed biotherapeutic. In an aspect, when the relative abundance of Corynebacterium sp. is less than about 50%, about 60%, about 70%, about 80%, or 90% of the microbiome, then a disclosed method can further comprise administering and/or continuing to administer a therapeutically effective amount of a disclosed biotherapeutic. In an aspect, when the relative abundance of Corynebacterium sp. is about 50% to about 60%, about 60% to about 70%, about 70% to about 80%, about 80% to about 90% of the microbiome, then a disclosed method can further comprise continuing to monitor the subject. In an aspect, when the relative abundance of Corynebacterium sp. is less than about 50%, about 60%, about 70%, about 80%, or 90%, then a disclosed method can further comprise continuing to monitor the subject.

In an aspect, a disclosed method can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect of a disclosed method of promoting respiratory health, techniques to monitor, measure, and/or assess the restoring one or more aspects of respiratory health can comprise qualitative (or subjective) means as well as quantitative (or objective) means. These means are known to the skilled person. For example, representative regulated variables and sensors relating to systemic homeostasis are provided below.

| Regulated Variable | Sensor |
|---|---|
| Blood Pressure/Blood | Aortic Body (Aorta), Carotid Body (Carotid |
| Volume/Na⁺ Conc./ | Artery), Atrial Volume Receptors (Heart), |
| | Juxtaglomerular Apparatus (Kidney) |
| Ca²⁺/Mg²⁺/PO₄³⁻ Conc. | Chief Cells (Parathyroid Gland) |
| Glucose | Islet of Langerhans (Pancreas) |
| Osmolarity | Circumventricular Organs (Hypothalamus) |
| pO₂, pCO₂, and pH | Aortic Body (Aorta), Carotid Body |
| | (Carotid Artery), Ventrolateral |
| | Medulla (Medulla) |
| Temperature | Thermosensory neurons (Skin), Preoptic |
| | Area (Hypothalamus) |

In an aspect of a disclosed method, a disclosed biotherapeutic, a disclosed secreted factor, a disclosed consortium of probiotics, a disclosed probiotic, or any combination thereof can be administered in the absence of identifying the one or more pathogenic bacteria and/or in the absence of characterizing the microbiome. In an aspect of a disclosed method, a disclosed biotherapeutic, a disclosed secreted factor, a disclosed consortium of probiotics, a disclosed probiotic, or any combination thereof can be administered on demand.

In an aspect of a disclosed method of promoting respiratory health, a disclosed biotherapeutic can be formulated for administration as a pharmaceutical formulation. In an aspect, a disclosed pharmaceutical formulation comprising a probiotic, a consortium of probiotics, factors secreted from a probiotic, factors secreted from a consortium of probiotics, or any combination thereof, and a pharmaceutically acceptable carrier and/or excipient can be used in any disclosed method of promoting respiratory health. Any pharmaceutical formulation disclosed herein can be used in a disclosed method of promoting respiratory health.

### E. Methods of Reducing the Risk of Developing an Infection

Disclosed herein is a method of reducing the risk of developing a pathogenic bacterial infection, the method comprising administering to a subject in need thereof a therapeutically effective amount of a biotherapeutic, thereby preventing and/or inhibiting the colonization of the pathogenic bacteria.

Disclosed herein is a method of reducing the risk of developing a S. pneumoniae infection, the method comprising administering to a subject in need thereof a therapeutically effective amount of a biotherapeutic, thereby preventing and/or inhibiting the colonization of S. pneumoniae.

In an aspect of a disclosed method of reducing the risk of developing a pathogenic bacterial and/or S. pneumoniae infection, a subject can be any subject. In an aspect, a subject that does not have an infection and/or is not suspected of having an infection.

In an aspect, following administration, the colonization of a pathogenic bacteria and/or S. pneumoniae in the subject's respiratory system can be inhibited and/or prevented.

In an aspect, a disclosed method of reducing the risk of developing a pathogenic bacterial and/or S. pneumoniae infection can further comprise promoting respiratory health in a subject.

In an aspect, a disclosed biotherapeutic can inhibit and/or prevent the growth of one or more pathogenic bacteria and/or S. pneumoniae in one or more parts of the subject's respiratory system.

In an aspect, a disclosed method of reducing the risk of developing a pathogenic bacterial and/or S. pneumoniae infection can further comprise characterizing the microbiome of a biological sample. In an aspect, a disclosed microbiome can comprise the nasopharyngeal microbiome, the nasal microbiome, or both.

In an aspect, a disclosed method of reducing the risk of developing a pathogenic bacterial and/or S. pneumoniae infection can further comprise obtaining a biological sample from the subject. In an aspect, a disclosed biological sample can comprise a nasal swab or lavage, a nasopharyngeal swab or lavage, or a pharyngeal swab or lavage, or any combination thereof. In an aspect, a disclosed biological sample can comprise nasal secretion and/or cough droplets.

In an aspect of a disclosed method of reducing the risk of developing a pathogenic bacterial and/or S. pneumoniae infection, characterizing the microbiome can comprise (i) collecting a biological sample from the subject; (ii) extracting nucleic acid from the subject's biological sample; and (iii) sequencing the extracted nucleic acid. In an aspect, sequencing the extracted nucleic acid can generate sequence data. In an aspect, characterizing the microbiome can further comprise analyzing the sequence data using taxonomic classification. In an aspect, using taxonomic classification can comprise PCR amplification. In an aspect, PCR amplification can comprise using primers targeting the 16S rRNA gene. In an aspect, PCR amplification can comprise primers targeting the V4 variable region of the 16S rRNA gene. In an aspect, disclosed primers can comprise a pair of primers. In an aspect, a disclosed pair of primers can comprise a primer having the sequence of SEQ ID NO:01 and a primer having the sequence of SEQ ID NO:02. In an aspect, a disclosed pair of primers can comprise a primer having the sequence of SEQ ID NO:03 and a primer having the sequence of SEQ ID NO:04. In an aspect, a disclosed pair of primers can comprise a primer having the sequence of SEQ ID NO:05 and a primer having the sequence of SEQ ID NO:06.

In an aspect, a disclosed method of reducing the risk of developing a pathogenic bacterial and/or S. pneumoniae infection can further comprise identifying the one or more pathogenic bacteria. In an aspect, the disclosed pathogenic bacteria can comprise Streptococcus sp. In an aspect, Streptococcus sp. can comprise S. pneumoniae. In an aspect, the disclosed pathogenic bacteria can comprise Staphylococcus sp. In an aspect, Staphylococcus sp. can comprise methicillin-sensitive S. aureus or methicillin-resistant S. aureus. In an aspect, the disclosed pathogenic bacteria can comprise Haemophilus influenzae or Moraxella catarrhalis.

In an aspect, a disclosed method of reducing the risk of developing a pathogenic bacterial and/or S. pneumoniae infection can further comprise diagnosing the subject with an infection. In an aspect, a diagnosed infection can comprise a respiratory infection. In an aspect, a diagnosed infection can affect the subject's mouth, nose, ears, sinuses, pharynx, trachea, bronchial tubes, lungs, alveoli, bronchioles, capillaries, lung lobes, pleura, cilia, epiglottis, larynx, or any combination thereof.

In an aspect, a disclosed method of reducing the risk of developing a pathogenic bacterial and/or S. pneumoniae infection can further comprise diagnosing the subject with pneumonia, otitis media, sinusitis, rhinosinusitis, or any combination thereof. In an aspect, otitis media (OM) can be considered a general term for middle-ear inflammation and can be classified clinically as acute otitis media (AOM), recurrent AOM, OM with effusion (OME), or chronic OME, chronic suppurative OM (CSOM), or middle ear effusion (MEE) (discussed supra).

In an aspect, sinusitis can occur when the tissue lining of the sinuses becomes swollen or inflamed. Sinusitis can comprise acute sinusitis (e.g., when symptoms are present for 4 weeks or less), chronic sinusitis (e.g., when the swelling of the sinuses is present for longer than 3 months), or subacute sinusitis (e.g., when the swelling is present between one and three months).

In an aspect, rhinosinusitis can occur when the mucosal lining in the paranasal sinuses and nasal cavity becomes inflamed. Most cases of acute rhinosinusitis are caused by viral upper respiratory infections.

In an aspect, a disclosed biotherapeutic can comprise a probiotic, a consortium of probiotics, factors secreted from a probiotic, factors secrete from a consortium of probiotics, or any combination thereof.

In an aspect, a disclosed biotherapeutic can comprise pharmaceutical formulation comprising a probiotic, a consortium of probiotics, factors secreted from a probiotic, factors secreted from a consortium of probiotics, or any combination thereof, and a pharmaceutically acceptable carrier and/or excipient. In an aspect, a disclosed pharmaceutical formulation can comprise at least one lyoprotectant. A disclosed lyoprotectant can comprise peptone, glycerol, lactose, gelatin, glucose, sucrose, trehalose, dextran, maltodextrin, adonitol, sodium glutamate, or any combination thereof.

In an aspect, a disclosed biotherapeutic can be lyophilized or freeze-dried such that, for example, a disclosed probiotic, a disclosed consortium of probiotics, disclosed factors secreted from a probiotic, disclosed factors secrete from a consortium of probiotics, or any combination thereof are all lyophilized or freeze-dried.

In an aspect, a disclosed biotherapeutic can comprise one or more probiotics. In an aspect, a disclosed consortium of probiotics can comprise at least one strain from the bacterial genus Corynebacterium, at least one strain from the bacterial genus Dolosigranulum, at least one strain from the bacterial genus Streptococcus, and at least one strain from the bacterial genus Lactobacillus.

In an aspect, the at least one strain from the bacterial genus Corynebacterium can comprise one or more strains of Corynebacterium accolens. In an aspect, the at least one strain from the bacterial genus Dolosigranulum can comprise one or more strains of Dolosigranulum pigrum. In an aspect, the at least one strain from the bacterial genus Streptococcus can comprise one or more strains of Streptococcus mitis, Streptococcus salivarius, Streptococcus oralis, or Streptococcus thermophilus. In an aspect, the at least one strain from the bacterial genus Lactobacillus can comprise one or more strains of Lactobacillus rhamnosus or Lactobacillus acidophilus.

In an aspect, one or more disclosed probiotics can comprise at least one strain from the bacterial genus one Corynebacterium sp. In an aspect, one or more disclosed probiotics can comprise at least one strain from the bacterial genus Corynebacterium and at least one strain from the bacterial genus Dolosigranulum. In an aspect, one or more disclosed probiotics can comprise at least one strain from the bacterial genus Corynebacterium, at least one strain from the bacterial genus Dolosigranulum, and at least one strain from the bacterial genus Streptococcus. In an aspect, one or more disclosed probiotics can comprise at least one strain from the bacterial genus Corynebacterium, at least one strain from the bacterial genus Dolosigranulum, and at least one strain from the bacterial genus Lactobacillus. In an aspect, one or more disclosed probiotics can comprise at least one strain from the bacterial genus Corynebacterium and at least one at least one strain from the bacterial genus Streptococcus. In an aspect, one or more disclosed probiotics can comprise at least one strain from the bacterial genus Corynebacterium, at least one strain from the bacterial genus Streptococcus, and at least one strain from the bacterial genus Lactobacillus. In an aspect, one or more probiotics can comprise at least one strain from the bacterial genus Corynebacterium and at least one strain from the bacterial genus Lactobacillus. In an aspect, one or more probiotics can comprise at least one strain from the bacterial genus Corynebacterium, at least one strain from the bacterial genus Dolosigranulum, at least one strain from the bacterial genus Streptococcus, and at least one strain from the bacterial genus Lactobacillus.

In an aspect of a disclosed method of reducing the risk of developing a pathogenic bacterial and/or S. pneumoniae infection, a disclosed biotherapeutic can comprise one or more factors secreted by a disclosed probiotic or secreted by a disclosed consortium of probiotics. In an aspect, a disclosed biotherapeutic can comprise one or more factors secreted by at least one strain from the bacterial genus Corynebacterium, at least one strain from the bacterial genus Dolosigranulum, at least one strain from the bacterial genus Streptococcus, and at least one strain from the bacterial genus Lactobacillus. In an aspect, a disclosed biotherapeutic can comprise one or more factors secreted by at least one strain from the bacterial genus Corynebacterium. In an aspect, a disclosed biotherapeutic can comprise one or more factors secreted by at least one strain from the bacterial genus Corynebacterium and at least one strain from the bacterial genus Dolosigranulum. In an aspect, a disclosed biotherapeutic can comprise one or more factors secreted by at least one strain from the bacterial genus Corynebacterium, at least one strain from the bacterial genus Dolosigranulum, and at least one train from the bacterial genus Streptococcus. In an aspect, a disclosed biotherapeutic can comprise one or more factors secreted by at least one strain from the bacterial genus Corynebacterium, at least one strain from the bacterial genus Dolosigranulum, and at least one strain from the bacterial genus Lactobacillus. In an aspect, a disclosed biotherapeutic can comprise one or more factors secreted by at least one strain from the bacterial genus Corynebacterium and at least one strain from the bacterial genus Streptococcus. In an aspect, a disclosed biotherapeutic can comprise comprises one or more factors secreted by at least one strain from the bacterial genus Corynebacterium, at least one strain from the bacterial genus Streptococcus, and at least one strain from the bacterial genus Lactobacillus. In an aspect, a disclosed biotherapeutic can comprise one or more factors secreted by at least one strain from the bacterial genus Corynebacterium and at least one strain from the bacterial genus Lactobacillus.

In an aspect, a disclosed Corynebacterium can comprise C. accolens, C. afermentans subsp. afermentans, C. afermentans subsp. lipophilum, C. ammoniagenes, C. amycolatum, C. appendicis, C. aquaticum, C. argentoratense, C. atypicum, C. aurimucosum, C. auris, C. bovis, C. canis, C. confusum, C. coyleae, C. diphtheriae, C. durum, C. efficiens, C. equi (now Rhodococcus equi), C. falsenii, C. flavescens, C. freiburgense, C. freneyi, C. glucuronolyticum, C. glutamicum, C. granulosum, C. haemolyticum, C. halofytica, C. kroppenstedtii, C. hansenii, C. imitans, C. jeikeium (group JK), C. kroppenstedtii, C. kutscheri, C. lipophiloflavum, C. macginleyi, C. massiliense, C. mastitidis-like, C. matruchotii, C. minutissimum, C. mucifaciens, C. mycetoides, C. ovis, C. parvum (Propionibacterium acnes), C. paurometabolum, C. pilbarense, C. propinquum, C. pseudodiphtheriticum (C. hofmannii), C. pseudotuberculosis, C. pyogenes-Trueperella pyogenes, C. pyruviciproducens, C. renale, C. resistans, C. riegelii, C. simulans, C. singular, C. spec, C. sputi, C. stationis, C. striatum, C. sundsvallense, C. tenuis, C. thomsenii, C. timonense, C. tuberculostearicum, C. tuscaniense, C. ulcerans, C. urealyticum (group D2), C. urealyticum, C. xerosis, Corynebacterium_BWA136, Corynebacterium_BWA297, Corynebacterium_DU041, Corynebacterium_DU044, or any combination thereof. In an aspect of a disclosed pharmaceutical formulation, a disclosed strain from the bacterial genus Corynebacterium can comprise C. accolens, C. coyleae, C. propinquum, C. pseudodiphtheriticum, C. amycolatum, C. glutamicum, C. aurimucosum, C. tuberculostearicum, C. afermentans, C. striatum, Corynebacterium_DU041, Corynebacterium_DU044, Corynebacterium_BWA136, Corynebacterium_BWA297, or any combination thereof. In an aspect, a disclosed strain from the bacterial genus Corynebacterium can comprise any species identified in Table 13.

In an aspect of a disclosed method of reducing the risk of developing a pathogenic bacterial and/or S. pneumoniae infection, a disclosed therapeutically effective dose can comprise a total of at least 100 bacterial CFUs of the one or more probiotics, a total of at least 200 to at least 300 bacterial CFUs of the one or more probiotics, a total of at least 300 to at least 400 bacterial CFUs of the one or more probiotics, a total of at least 400 to at least 500 bacterial CFUs of the one or more probiotics, a total of at least 500 to at least 600 bacterial CFUs of the one or more probiotics, a total of at least 600 to at least 700 bacterial CFUs of the one or more probiotics, a total of at least 700 to at least 800 bacterial CFUs of the one or more probiotics, a total of at least 800 to at least 900 bacterial CFUs of the one or more probiotics, a total of at least 900 to at least 1000 bacterial CFUs of the one or more probiotics, or a total of at least 1000 bacterial CFUs of one or more disclosed probiotics (such as, for example, Corynebacterium sp., Dolosigranulum sp., Streptococcus sp., Lactobacillus sp., or any combination thereof).

In an aspect of a disclosed method of reducing the risk of developing a pathogenic bacterial and/or S. pneumoniae infection, a disclosed therapeutically effective dose can comprise a total of at least 1000 to at least 2000 bacterial CFUs of the one or more probiotics, a total of at least 2000 to at least 3000 bacterial CFUs of the one or more probiotics, a total of at least 3000 to at least 4000 bacterial CFUs of the one or more probiotics, a total of at least 4000 to at least 5000 bacterial CFUs of the one or more probiotics, a total of at least 5000 to at least 6000 bacterial CFUs of the one or more probiotics, a total of at least 6000 to at least 7000 bacterial CFUs of the one or more probiotics, a total of at least 8000 to at least 9000 bacterial CFUs of the one or more probiotics, or a total of at least 10,000 bacterial CFUs of one or more disclosed probiotics (such as, for example, Corynebacterium sp., Dolosigranulum sp., Streptococcus sp., Lactobacillus sp., or any combination thereof).

In an aspect of a disclosed method of reducing the risk of developing a pathogenic bacterial and/or S. pneumoniae infection, a disclosed therapeutically effective dose can comprise a total of at least 100 bacterial CFUs of Corynebacterium sp., Dolosigranulum sp., Streptococcus sp., Lactobacillus sp., or any combination thereof, a total of at least 200 to at least 300 bacterial CFUs of Corynebacterium sp., Dolosigranulum sp., Streptococcus sp., Lactobacillus sp., or any combination thereof, a total of at least 300 to at least 400 bacterial CFUs of Corynebacterium sp., Dolosigranulum sp., Streptococcus sp., Lactobacillus sp., or any combination thereof, a total of at least 400 to at least 500 bacterial CFUs of Corynebacterium sp., Dolosigranulum sp., Streptococcus sp., Lactobacillus sp., or any combination thereof, a total of at least 500 to at least 600 bacterial CFUs of Corynebacterium sp., Dolosigranulum sp., Streptococcus sp., Lactobacillus sp., or any combination thereof, a total of at least 600 to at least 700 bacterial CFUs of Corynebacterium sp., Dolosigranulum sp., Streptococcus sp., Lactobacillus sp., or any combination thereof, a total of at least 700 to at least 800 bacterial CFUs of Corynebacterium sp., Dolosigranulum sp., Streptococcus sp., Lactobacillus sp., or any combination thereof, a total of at least 800 to at least 900 bacterial CFUs of Corynebacterium sp., Dolosigranulum sp., Streptococcus sp., Lactobacillus sp., or any combination thereof, a total of at least 900 to at least 1000 bacterial CFUs of Corynebacterium sp., Dolosigranulum sp., Streptococcus sp., Lactobacillus sp., or any combination thereof, or a total of at least 1000 bacterial CFUs of Corynebacterium sp., Dolosigranulum sp., Streptococcus sp., Lactobacillus sp., or any combination thereof.

In an aspect of a disclosed method of reducing the risk of developing a pathogenic bacterial and/or S. pneumoniae infection, a disclosed therapeutically effective dose can comprise a total of at least 1000 to at least 2000 bacterial CFUs of Corynebacterium sp., Dolosigranulum sp., Streptococcus sp., Lactobacillus sp., or any combination thereof, a total of at least 2000 to at least 3000 bacterial CFUs of Corynebacterium sp., Dolosigranulum sp., Streptococcus sp., Lactobacillus sp., or any combination thereof, a total of at least 3000 to at least 4000 bacterial CFUs of Corynebacterium sp., Dolosigranulum sp., Streptococcus sp., Lactobacillus sp., or any combination thereof, a total of at least 4000 to at least 5000 bacterial CFUs of the Corynebacterium sp., Dolosigranulum sp., Streptococcus sp., Lactobacillus sp., or any combination thereof, a total of at least 5000 to at least 6000 bacterial CFUs of Corynebacterium sp., Dolosigranulum sp., Streptococcus sp., Lactobacillus sp., or any combination thereof, a total of at least 6000 to at least 7000 bacterial CFUs of Corynebacterium sp., Dolosigranulum sp., Streptococcus sp., Lactobacillus sp., or any combination thereof, a total of at least 8000 to at least 9000 bacterial CFUs of Corynebacterium sp., Dolosigranulum sp., Streptococcus sp., Lactobacillus sp., or any combination thereof, or a total of at least 10,000 bacterial CFUs of Corynebacterium sp., Dolosigranulum sp., Streptococcus sp., Lactobacillus sp., or any combination thereof.

In an aspect of a disclosed method of reducing the risk of developing a pathogenic bacterial and/or S. pneumoniae infection, a disclosed therapeutically effective dose can comprise a total of at least 10⁴ to at least 10⁵ bacterial CFUs of the one or more probiotics, a total of at least 10⁵ to at least 10⁶ bacterial CFUs of the one or more probiotics, a total of at least 10⁶ to at least 10⁷ bacterial CFUs of the one or more probiotics, a total of at least 10⁷ to at least 10⁸ bacterial CFUs of the one or more probiotics, a total of at least 10⁸ to at least 10⁹ bacterial CFUs of the one or more probiotics, a total of at least 10⁹ to at least 10¹⁰ bacterial CFUs of the one or more probiotics, a total of at least 10¹⁰ to at least 10¹¹ bacterial CFUs of the one or more probiotics, a total of at least 10¹¹ to at least 10¹² bacterial CFUs of the one or more probiotics, a total of at least 10¹² bacterial CFUs of the one or more probiotics, or more than 10¹² bacterial CFUs of one or more disclosed probiotics (such as, for example, Corynebacterium sp., Dolosigranulum sp., Streptococcus sp., Lactobacillus sp., or any combination thereof).

In an aspect, a disclosed therapeutically effective dose can comprise at least 10³, 10⁴, 10⁵, 10⁶, 10⁷, 10⁸, 10⁹, 10¹⁰, 10¹¹, or 10¹² CFUs of a disclosed probiotic or a consortium of disclosed probiotics, preferably at least 1.2×10³, 1.4×10³, 2×10³, or 3×10³ CFUs of a disclosed probiotic or a consortium of disclosed probiotics, at least 5×10³, 5.02×10³, 5.04×10³, 5.2×10³, or 5.4×10³ CFUs of a disclosed probiotic or a consortium of disclosed probiotics, at least 1.02×10⁴, 1.04×10⁴, 1.2×10⁴, 1.4×10⁴, 1.5×10⁴, 2×10⁴, or 3×10⁴ CFUs of a disclosed probiotic or a consortium of disclosed probiotics, at least 5×10⁴, 5.02×10⁴, 5.04×10⁴, 5.2×10⁴, or 5.4×10⁴ CFUs of a disclosed probiotic or a consortium of disclosed probiotics, at least 1.02×10⁵, 1.04×10⁵, 1.2×10⁵, 1.4×10⁵, 1.5×10⁵, 2×10⁵, or 3×10⁵ CFUs of a disclosed probiotic or a consortium of disclosed probiotics, at least 1.02×10⁶, 1.04×10⁶, 1.2×10⁶, 1.4×10⁶, 2×10⁶, or 3×10⁶ CFUs of a disclosed probiotic or a consortium of disclosed probiotics, at least 1.02×10⁷, 1.04×10⁷, 1.2×10⁷, 1.4×10⁷, 2×10⁷, or 3×10⁷ CFUs of a disclosed probiotic or a consortium of disclosed probiotics, at least 1.02×10⁸, 1.04×10⁸, 1.2×10⁸, 1.4×10⁸, 2×10⁸, or 3×10⁸ CFUs of a disclosed probiotic or a consortium of disclosed probiotics, at least 1.02×10⁹, 1.04×10⁹, 1.2×10⁹, 1.4×10⁹, 2×10⁹, or 3×10⁹ CFUs of a disclosed probiotic or a consortium of disclosed probiotics, at least 10¹⁰ CFUs of a disclosed probiotic or a consortium of disclosed probiotics, at least 1.02×10¹⁰, 1.04×10¹⁰, 1.2×10¹⁰, 1.4×10¹⁰, 2×10¹⁰, or 3×10¹⁰ CFUs of a disclosed probiotic or a consortium of disclosed probiotics, at least 1.02×10¹¹, 1.04×10¹¹, 1.2×10¹¹, 1.4×10¹¹, 2×10¹¹, or 3×10¹¹ CFUs of a disclosed probiotic or a consortium of disclosed probiotics, or at least 1.02×10¹², 1.04×10¹², 1.2×10¹², 1.4×10¹², 2×10¹², or 3×10¹² CFUs of a disclosed probiotic or a consortium of disclosed probiotics per dose.

In an aspect, a disclosed therapeutically effective dose can comprise any amount or an unlimited amount of bacterial CFUs of the one or more probiotics.

In an aspect of a disclosed method of reducing the risk of developing a pathogenic bacterial and/or S. pneumoniae infection, a disclosed therapeutically effective dose can comprise Corynebacterium sp., Dolosigranulum sp., Streptococcus sp., and Lactobacillus sp. in a ratio of about 1:0.01:0.01:0.01 to about 1:1:1:1. In an aspect, a disclosed therapeutically effective dose can comprise Corynebacterium sp., Dolosigranulum sp., Streptococcus sp., and Lactobacillus sp. in any ratio. In an aspect of a disclosed pharmaceutical formulation, a disclosed therapeutically effective dose can comprise a higher dose of Corynebacterium sp., or a higher dose of Dolosigranulum sp., or a higher dose of Streptococcus sp., or a higher dose of Lactobacillus sp. as compared to any other bacteria in the consortium. In an aspect, a disclosed therapeutically effective dose can comprise a dose of Lactobacillus sp. that is about double that of Corynebacterium sp.

In an aspect, a disclosed method of reducing the risk of developing a pathogenic bacterial and/or S. pneumoniae infection can further comprise treating the subject. In an aspect, treating the subject can comprise treating the subject's respiratory infection. In an aspect, treating the subject can comprise treating the subject's non-respiratory infection.

In an aspect, a disclosed method of reducing the risk of developing a pathogenic bacterial and/or S. pneumoniae infection can comprise further comprising administering to the subject a therapeutically effective amount of one or more anti-bacterial agents. Anti-bacterial agents and combinations of anti-bacterial agents are known to the art and discussed supra in Part VII(C)(1).

In an aspect, a disclosed method of reducing the risk of developing a pathogenic bacterial and/or S. pneumoniae infection can comprise further comprising administering to the subject a therapeutically effective amount of (i) one or more active agents, (ii) one or more biologically active agents, (iii) one or more pharmaceutically active agents, (iv) one or more immune-based therapeutic agents, (v) one or more clinically approved agents, or (vi) any combination thereof. In an aspect, a therapeutically effective amount of an active agent, a biologically active agent, a pharmaceutically active agent, an immune-based therapeutic active agent, a clinically approved agent, or an anti-bacterial agent can be about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method of reducing the risk of developing a pathogenic bacterial and/or S. pneumoniae infection can further comprise increasing the relative abundance of one or more probiotics in the subject's microbiome. In an aspect, a disclosed method of reducing the risk of developing a pathogenic bacterial and/or S. pneumoniae infection can further comprise increasing the relative abundance of one or more of Corynebacterium sp., Dolosigranulum sp., Streptococcus sp., and Lactobacillus sp. in the subject's microbiome. In an aspect, a disclosed method of reducing the risk of developing a pathogenic bacterial and/or S. pneumoniae infection can further comprise increasing the relative abundance of one or more of at least one strain from the bacterial genus Corynebacterium, at least one strain from the bacterial genus Dolosigranulum, at least one strain from the bacterial genus Streptococcus, and at least one strain from the bacterial genus Lactobacillus, or any combination thereof in the subject's microbiome. In an aspect, a disclosed method of reducing the risk of developing a pathogenic bacterial and/or S. pneumoniae infection can further comprise increasing the relative abundance of at least one strain from the bacterial genus Corynebacterium in the subject's microbiome. In an aspect, a disclosed method of reducing the risk of developing a pathogenic bacterial and/or S. pneumoniae infection can further comprise increasing the relative abundance of at least one strain from the bacterial genus Dolosigranulum in the subject's microbiome. In an aspect, a disclosed method of reducing the risk of developing a pathogenic bacterial and/or S. pneumoniae infection can further comprise increasing the relative abundance of at least one strain from the bacterial genus Streptococcus in the subject's microbiome. In an aspect, a disclosed method of reducing the risk of developing a pathogenic bacterial and/or S. pneumoniae infection can further comprise increasing the relative abundance of at least one strain from the bacterial genus Lactobacillus in the subject's microbiome.

In an aspect, a disclosed method of reducing the risk of developing a pathogenic bacterial and/or S. pneumoniae infection can further comprise decreasing the relative abundance of one or more pathogenic bacteria in the subject's microbiome. In an aspect, a disclosed method of reducing the risk of developing a pathogenic bacterial and/or S. pneumoniae infection can further comprise decreasing the relative abundance of S. pneumoniae in the subject's microbiome. In an aspect, a disclosed method of reducing the risk of developing a pathogenic bacterial and/or S. pneumoniae infection can further comprise decreasing the relative abundance of a S. aureus in the subject's microbiome. In an aspect, a disclosed method of reducing the risk of developing a pathogenic bacterial and/or S. pneumoniae infection can further comprise decreasing the relative abundance of Haemophilus influenzae and/or Moraxella catarrhalis in the subject's microbiome.

In an aspect, a disclosed method of reducing the risk of developing a pathogenic bacterial and/or S. pneumoniae infection can further comprise administering to the subject one or more therapeutic agents. In an aspect, a disclosed therapeutic agent can comprise a biologically active agent, a pharmaceutically active agent, an anti-bacterial agent, an anti-fungal agent, an anti-viral agent, a corticosteroid, an analgesic, an immunostimulant, an immune-based product, or any combination thereof. In an aspect of a disclosed method of promoting respiratory health, administering can comprise intranasal administration, oral administration, sublingual administration, or any combination thereof.

In an aspect of a disclosed method of reducing the risk of developing a pathogenic bacterial and/or S. pneumoniae infection, a subject can be a healthy subject, for example, a subject that does not have an infection and/or is not suspected of having an infection.

In an aspect, a subject can be an adult, a child, or an infant. In an aspect, a subject can be a neonate. In an aspect, a subject can be a premature infant. In an aspect, a subject can be immune-compromised. In an aspect, a subject can have diabetes or a chronic disease (e.g., heart disease, kidney disease, or liver disease). In an aspect, a subject can have HIV. In an aspect, a subject can have cancer or has had cancer. In an aspect, a subject can be the recipient of one or more solid organ transplants. In an aspect, a subject can have nephrotic syndrome. In an aspect, a subject can be alcoholic or can smoke cigarettes. In an aspect, a subject can have a chronic lung disease. In an aspect, a disclosed chronic lung disease can comprise emphysema, chronic obstructive lung disease, chronic obstructive pulmonary disease, asthma, or any combination thereof.

In an aspect, a subject can be diagnosed with an infection of one or more parts of the respiratory system. In an aspect, a subject can be suspected of having an infection in one or more parts of the respiratory system. In an aspect, a disclosed therapeutically effective amount of a biotherapeutic can comprise an amount sufficient to inhibit and/or prevent the growth of one or more pathogenic bacteria in one or more parts of the subject's respiratory system. In an aspect, a disclosed therapeutically effective amount of a biotherapeutic can comprise an amount sufficient to inhibit and/or prevent the spread of one or more pathogenic bacteria from a first part of the subject's respiratory system to a second part of the subject's respiratory system.

In an aspect, one or more disclosed probiotics can inhibit and/or prevent the growth and/or spread of one or more pathogenic bacteria using competitive adherence and/or non-adherence mechanisms (e.g., secretion of antimicrobial factors). In an aspect, one or more disclosed probiotics can inhibit and/or prevent the adherence and/or colonization of one or more pathogenic bacteria. In an aspect, one or more disclosed probiotics can modulate the endocytosis or paracellular migration of one or more pathogenic bacteria. In an aspect, one or more disclosed probiotics can inhibit and/or prevent the spread of one or more pathogenic bacteria by competitive adherence and/or niche occupancy.

In an aspect, one or more disclosed probiotics can inhibit and/or prevent the growth and/or spread of S. pneumoniae using competitive adherence and/or non-adherence mechanisms (e.g., secretion of antimicrobial factors). In an aspect, one or more disclosed probiotics can inhibit and/or prevent the adherence and/or colonization of S. pneumoniae. In an aspect, one or more disclosed probiotics can modulate the endocytosis or paracellular migration of S. pneumoniae. In an aspect, one or more disclosed probiotics can inhibit and/or prevent the spread S. pneumoniae by competitive adherence and/or niche occupancy.

In an aspect of a disclosed method of reducing the risk of developing a pathogenic bacterial and/or S. pneumoniae infection, one or more disclosed probiotics can secrete one or more factors. In an aspect, a disclosed secreted factor can be species-dependent and/or strain-dependent. In an aspect, a disclosed secreted factor can inhibit and/or prevent the growth and/or spread of one or more pathogenic bacteria. In an aspect, a disclosed secreted factor can inhibit and/or prevent the adherence and/or colonization of one or more pathogenic bacteria. In an aspect, a disclosed secreted factor can modulate the endocytosis or paracellular migration of one or more pathogenic bacteria. In an aspect, a disclosed secreted factor can inhibit and/or prevent the spread of one or more pathogenic bacteria by competitive adherence and/or niche occupancy.

In an aspect of a disclosed method of reducing the risk of developing a pathogenic bacterial and/or S. pneumoniae infection, a disclosed secreted factor can inhibit and/or prevent the growth and/or spread of S. pneumoniae. In an aspect, a disclosed secreted factor can inhibit and/or prevent the adherence and/or colonization of S. pneumoniae. In an aspect, a disclosed secreted factor can modulate the endocytosis or paracellular migration of S. pneumoniae. In an aspect, a disclosed secreted factor can inhibit and/or prevent the spread of S. pneumoniae by competitive adherence and/or niche occupancy.

In an aspect of a disclosed method of reducing the risk of developing a pathogenic bacterial and/or S. pneumoniae infection, Corynebacterium sp., Dolosigranulum sp., Streptococcus sp., Lactobacillus sp., or any combination thereof can secrete one or more factors that inhibit and/or prevent the growth and/or spread of one or more pathogenic bacteria. In an aspect of a disclosed method of reducing the risk of developing a pathogenic bacterial and/or S. pneumoniae infection, Corynebacterium sp., Dolosigranulum sp., Streptococcus sp., Lactobacillus sp., or any combination thereof can secrete one or more factors that inhibit and/or prevent the adherence and/or colonization of one or more pathogenic bacteria. In an aspect of a disclosed method of reducing the risk of developing a pathogenic bacterial and/or S. pneumoniae infection, Corynebacterium sp., Dolosigranulum sp., Streptococcus sp., Lactobacillus sp., or any combination thereof can secrete one or more secreted factors that modulate the endocytosis or paracellular migration of one or more pathogenic bacteria. In an aspect of a disclosed method of reducing the risk of developing a pathogenic bacterial and/or S. pneumoniae infection, Corynebacterium sp., Dolosigranulum sp., Streptococcus sp., Lactobacillus sp., or any combination thereof can secrete one or more secreted factors inhibit and/or prevent the spread of one or more pathogenic bacteria by competitive adherence and/or niche occupancy. In an aspect of a disclosed method of reducing the risk of developing a pathogenic bacterial and/or S. pneumoniae infection, Corynebacterium sp., Dolosigranulum sp., Streptococcus sp., Lactobacillus sp., or any combination thereof can secrete one or more secreted factors inhibit and/or prevent the growth and/or spread of S. pneumoniae. In an aspect of a disclosed method of reducing the risk of developing a pathogenic bacterial and/or S. pneumoniae infection, Corynebacterium sp., Dolosigranulum sp., Streptococcus sp., Lactobacillus sp., or any combination thereof can secrete one or more factors inhibit and/or prevent the adherence and/or colonization of S. pneumoniae. In an aspect, a disclosed method of reducing the risk of developing a pathogenic bacterial and/or S. pneumoniae infection, Corynebacterium sp., Dolosigranulum sp., Streptococcus sp., Lactobacillus sp., or any combination thereof secrete one or more factors can modulate the endocytosis or paracellular migration of S. pneumoniae. In an aspect of a disclosed method of reducing the risk of developing a pathogenic bacterial and/or S. pneumoniae infection, Corynebacterium sp., Dolosigranulum sp., Streptococcus sp., Lactobacillus sp., or any combination thereof can secrete one or more factors inhibit and/or prevent the spread of S. pneumoniae by competitive adherence and/or niche occupancy.

In an aspect of a disclosed method of reducing the risk of developing a pathogenic bacterial and/or S. pneumoniae infection, Corynebacterium sp. can secrete one or more factors that inhibit and/or prevent the growth and/or spread of one or more pathogenic bacteria. In an aspect of a disclosed method of reducing the risk of developing a pathogenic bacterial and/or S. pneumoniae infection, Corynebacterium sp. can secrete one or more factors that inhibit and/or prevent the adherence and/or colonization of one or more pathogenic bacteria. In an aspect of a disclosed method of reducing the risk of developing a pathogenic bacterial and/or S. pneumoniae infection, Corynebacterium sp. can secrete one or more secreted factors that modulate the endocytosis or paracellular migration of one or more pathogenic bacteria. In an aspect of a disclosed method of reducing the risk of developing a pathogenic bacterial and/or S. pneumoniae infection, Corynebacterium sp. can secrete one or more secreted factors that inhibit and/or prevent the spread of one or more pathogenic bacteria by competitive adherence and/or niche occupancy.

In an aspect of a disclosed method of reducing the risk of developing a pathogenic bacterial and/or S. pneumoniae infection, Corynebacterium sp. can secrete one or more secreted factors that inhibit and/or prevent the growth and/or spread of S. pneumoniae. In an aspect of a disclosed method of reducing the risk of developing a pathogenic bacterial and/or S. pneumoniae infection, Corynebacterium sp. can secrete one or more factors that inhibit and/or prevent the adherence and/or colonization of S. pneumoniae. In an aspect of a disclosed method of reducing the risk of developing a pathogenic bacterial and/or S. pneumoniae infection, Corynebacterium sp. can secrete one or more factors that modulate the endocytosis or paracellular migration of S. pneumoniae. In an aspect of a disclosed method of reducing the risk of developing a pathogenic bacterial and/or S. pneumoniae infection, Corynebacterium sp. can secrete one or more factors that inhibit and/or prevent the spread of S. pneumoniae by competitive adherence and/or niche occupancy.

In an aspect, a disclosed method of reducing the risk of developing a pathogenic bacterial and/or S. pneumoniae infection can comprise a 10%, 20%, 30%, 40%, 50%, 60%, 70%, 80%, 90%, 100%, or any amount of inhibiting and/or preventing the growth and/or spread of one or more pathogenic bacteria (e.g., S. pneumoniae) when compared to a control subject (such as, for example, a subject that has not received a disclosed biotherapeutic). In an aspect, a disclosed method of reducing the risk of developing a pathogenic bacterial and/or S. pneumoniae infection can comprise a 10-20%, 20-30%, 30-40%, 40-50%, 50-60%, 60-70%, 70-80%, 80-90%, or 90-100% or any amount of inhibiting and/or preventing the growth and/or spread of one or more pathogenic bacteria (e.g., S. pneumoniae) when compared to a control subject (such as, for example, a subject that has not received a disclosed biotherapeutic).

In an aspect, a disclosed method of reducing the risk of developing a pathogenic bacterial and/or S. pneumoniae infection can comprise a 10%, 20%, 30%, 40%, 50%, 60%, 70%, 80%, 90%, 100%, or any amount of inhibiting and/or preventing the adherence and/or colonization of one or more pathogenic bacteria (e.g., S. pneumoniae) when compared to a control subject (such as, for example, a subject that has not received a disclosed biotherapeutic). In an aspect, a disclosed method of reducing the risk of developing a pathogenic bacterial and/or S. pneumoniae infection can comprise a 10-20%, 20-30%, 30-40%, 40-50%, 50-60%, 60-70%, 70-80%, 80-90%, or 90-100% or any amount of inhibiting and/or preventing the adherence and/or colonization of one or more pathogenic bacteria (e.g., S. pneumoniae) when compared to a control subject (such as, for example, a subject that has not received a disclosed biotherapeutic).

In an aspect, a disclosed method of reducing the risk of developing a pathogenic bacterial and/or S. pneumoniae infection can comprise a 10%, 20%, 30%, 40%, 50%, 60%, 70%, 80%, 90%, 100%, or any amount of modulating the endocytosis or paracellular migration of one or more pathogenic bacteria (e.g., S. pneumoniae) when compared to a control subject (such as, for example, a subject that has not received a disclosed biotherapeutic). In an aspect, a disclosed method of reducing the risk of developing a pathogenic bacterial and/or S. pneumoniae infection can comprise a 10-20%, 20-30%, 30-40%, 40-50%, 50-60%, 60-70%, 70-80%, 80-90%, or 90-100% or any amount of modulating the endocytosis or paracellular migration of one or more pathogenic bacteria (e.g., S. pneumoniae) when compared to a control subject (such as, for example, a subject that has not received a disclosed biotherapeutic).

In an aspect, a disclosed method of reducing the risk of developing a pathogenic bacterial and/or S. pneumoniae infection can comprise a 10%, 20%, 30%, 40%, 50%, 60%, 70%, 80%, 90%, 100%, or any amount of inhibiting and/or preventing the spread of one or more pathogenic bacteria (e.g., S. pneumoniae) by competitive adherence and/or niche occupancy when compared to a control subject (such as, for example, a subject that has not received a disclosed biotherapeutic). In an aspect, a disclosed method of reducing the risk of developing a pathogenic bacterial and/or S. pneumoniae infection can comprise a 10-20%, 20-30%, 30-40%, 40-50%, 50-60%, 60-70%, 70-80%, 80-90%, or 90-100% or any amount of inhibiting and/or preventing the spread of one or more pathogenic bacteria (e.g., S. pneumoniae) by competitive adherence and/or niche occupancy when compared to a control subject (such as, for example, a subject that has not received a disclosed biotherapeutic).

In an aspect, a disclosed method of reducing the risk of developing a pathogenic bacterial and/or S. pneumoniae infection can comprise repeating the administering of one or more disclosed biotherapeutics, one or more disclosed secreted factors, a disclosed consortium of probiotics, one or more disclosed probiotics, or any combination thereof. For example, in an aspect, repeating the administering can comprise administering one or more times daily (e.g., 1, 2, 3, or 4 times). In an aspect, repeating the administering can comprise administering on several consecutive days (e.g., for 1, 2, 3, 4, 5, 6, 7, 8, 9, or 10 days, or more than 10 days), weeks (e.g., for 1, 2, 3, 4, 5, 6, 7, 8, 9, or 10 weeks, or more than 10 weeks), or months (e.g., for 1, 2, 3, 4, 5, or 6 months, or more than 6 months). In an aspect, a disclosed biotherapeutic, a disclosed secreted factor, a disclosed consortium of probiotics, a disclosed probiotic, or any combination thereof can be administered 1-3 times per day for about 7 to 21 days.

In an aspect, a disclosed method of reducing the risk of developing a pathogenic bacterial and/or S. pneumoniae infection can comprise monitoring the subject. In an aspect, monitoring the subject can comprise monitoring the subject for the development of adverse effects. In an aspect, in the absence of adverse effects, a disclosed method of reducing the risk of developing a pathogenic bacterial and/or S. pneumoniae infection can comprise continuing to treat the subject. In an aspect, continuing to treat the subject can comprise continuing to administer to the subject one or more disclosed biotherapeutics, one or more disclosed secreted factors, a disclosed consortium of probiotics, one or more disclosed probiotics, or any combination thereof. In an aspect, in the presence of adverse effects, a disclosed method of reducing the risk of developing a pathogenic bacterial and/or S. pneumoniae infection can comprise modifying one or more steps of the method. In an aspect, modifying one or more steps of a disclosed method can comprise modifying the administering step. In an aspect, modifying the administering step can comprise changing the amount of one or more disclosed biotherapeutics, one or more disclosed secreted factors, a disclosed consortium of probiotics, one or more disclosed probiotics, or any combination thereof administered to the subject, changing the frequency of administration of one or more disclosed biotherapeutics, one or more disclosed secreted factors, a disclosed consortium of probiotics, one or more disclosed probiotics, or any combination thereof, changing the duration of administration of one or more disclosed biotherapeutics, one or more disclosed secreted factors, a disclosed consortium of probiotics, one or more disclosed probiotics, or any combination thereof, changing the route of administration of one or more disclosed biotherapeutics, one or more disclosed secreted factors, a disclosed consortium of probiotics, one or more disclosed probiotics, or any combination thereof, or any combination of disclosed changes.

In an aspect, a disclosed method of reducing the risk of developing a pathogenic bacterial and/or S. pneumoniae infection can comprise manipulating the microbiome to inhibit and/or prevent infection caused by one or more pathogenic bacteria, such as an infection caused by S. pneumoniae. In an aspect, a disclosed method of reducing the risk of developing a pathogenic bacterial and/or S. pneumoniae infection can comprise manipulating the microbiome to decrease and/or minimize the risk of infection caused by one or more pathogenic bacteria, such as an infection caused by S. pneumoniae. For example, in an aspect, when the relative abundance of one or more disclosed Corynebacterium sp. is below a threshold level of 1.2×10⁶ 16S rRNA gene copies per biological sample, then the risk of colonization by S. pneumoniae is increased and/or higher and/or enhanced. In an aspect, when the relative abundance of Corynebacterium sp. is below a threshold level of 1.2×10⁶ 16S rRNA gene copies per biological sample, then a disclosed method can further comprise administering and/or continuing to administer a therapeutically effective amount of a disclosed biotherapeutic. In an aspect, when the relative abundance of Corynebacterium sp. is above 1.2×10⁶ 16S rRNA gene copies per biological sample, then a disclosed method can further comprise continuing to monitor the subject.

For example, in an aspect, when the relative abundance of one or more disclosed Corynebacterium sp. is less than about 50%, about 60%, about 70%, about 80%, or 90% of the microbiome, then the risk of colonization by S. pneumoniae is increased and/or higher and/or enhanced. For example, in an aspect, when the relative abundance of one or more disclosed Corynebacterium sp. is about 50% to about 60%, about 60% to about 70%, about 70% to about 80%, about 80% to about 90% of the microbiome, then the risk of colonization by S. pneumoniae is increased and/or higher and/or enhanced. In an aspect, when the relative abundance of Corynebacterium sp. is about 50% to about 60%, about 60% to about 70%, about 70% to about 80%, about 80% to about 90% of the microbiome, then a disclosed method can further comprise administering and/or continuing to administer a therapeutically effective amount of a disclosed biotherapeutic. In an aspect, when the relative abundance of Corynebacterium sp. is less than about 50%, about 60%, about 70%, about 80%, or 90% of the microbiome, then a disclosed method can further comprise administering and/or continuing to administer a therapeutically effective amount of a disclosed biotherapeutic. In an aspect, when the relative abundance of Corynebacterium sp. is about 50% to about 60%, about 60% to about 70%, about 70% to about 80%, about 80% to about 90% of the microbiome, then a disclosed method can further comprise continuing to monitor the subject. In an aspect, when the relative abundance of Corynebacterium sp. is less than about 50%, about 60%, about 70%, about 80%, or 90%, then a disclosed method can further comprise continuing to monitor the subject.

In an aspect, a disclosed method of reducing the risk of developing a pathogenic bacterial and/or S. pneumoniae infection can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect of a disclosed method of reducing the risk of developing a pathogenic bacterial and/or S. pneumoniae infection, techniques to monitor, measure, and/or assess the restoring one or more aspects of respiratory health can comprise qualitative (or subjective) means as well as quantitative (or objective) means. These means are known to the skilled person. For example, representative regulated variables and sensors relating to systemic homeostasis are discussed supra.

In an aspect of a disclosed method of reducing the risk of developing a pathogenic bacterial and/or S. pneumoniae infection, a disclosed biotherapeutic, a disclosed secreted factor, a disclosed consortium of probiotics, a disclosed probiotic, or any combination thereof can be administered in the absence of identifying the one or more pathogenic bacteria and/or in the absence of characterizing the microbiome. In an aspect of a disclosed method, a disclosed biotherapeutic, a disclosed secreted factor, a disclosed consortium of probiotics, a disclosed probiotic, or any combination thereof can be administered on demand.

In an aspect of a disclosed method of reducing the risk of developing a pathogenic bacterial and/or S. pneumoniae infection, a disclosed biotherapeutic can be formulated for administration as a pharmaceutical formulation. In an aspect, a disclosed pharmaceutical formulation comprising a probiotic, a consortium of probiotics, factors secreted from a probiotic, factors secreted from a consortium of probiotics, or any combination thereof, and a pharmaceutically acceptable carrier and/or excipient can be used in any disclosed method of reducing the risk of developing a pathogenic bacterial and/or S. pneumoniae infection. Any pharmaceutical formulation disclosed herein can be used in a disclosed method of reducing the risk of developing a pathogenic bacterial and/or S. pneumoniae infection.

### F. Methods of Treating S. pneumoniae Colonization and/or Infection

Disclosed herein is a method of treating S. pneumoniae colonization and/or infection in a subject, the method comprising administering a therapeutically effective amount of a biotherapeutic to the subject in need thereof, wherein the colonization of S. pneumoniae in the subject's respiratory system is inhibited and/or prevented following administration.

Disclosed herein is a method of treating S. pneumoniae colonization in a subject, the method comprising administering a therapeutically effective amount of a biotherapeutic to the subject in need thereof, thereby inhibiting and/or preventing colonization of S. pneumoniae in the subject's respiratory system.

In an aspect, following administration, the colonization and/or infection of S. pneumoniae in the subject's respiratory system can be inhibited and/or prevented.

In an aspect, following administration, the spread of colonization and/or the spread infection of S. pneumoniae in the subject's respiratory system can be inhibited and/or prevented.

In an aspect, following administration, the risk of developing a S. pneumoniae infection in one or more parts in the subject's respiratory system can be inhibited and/or prevented.

In an aspect, a disclosed method of treating S. pneumoniae colonization and/or infection can further comprise promoting respiratory health in a subject.

In an aspect, a disclosed biotherapeutic can inhibit and/or prevent the growth of S. pneumoniae in one or more parts of the subject's respiratory system.

In an aspect, a disclosed method of treating S. pneumoniae colonization and/or infection can further comprise characterizing the microbiome of a biological sample. In an aspect, a disclosed microbiome can comprise the nasopharyngeal microbiome, the nasal microbiome, or both.

In an aspect, a disclosed method of treating S. pneumoniae colonization and/or infection can further comprise obtaining a biological sample from the subject. In an aspect, a disclosed biological sample can comprise a nasal swab or lavage, a nasopharyngeal swab or lavage, or a pharyngeal swab or lavage, or any combination thereof. In an aspect, a disclosed biological sample can comprise nasal secretion and/or cough droplets.

In an aspect of a disclosed method of treating S. pneumoniae colonization and/or infection, characterizing the microbiome can comprise (i) collecting a biological sample from the subject; (ii) extracting nucleic acid from the subject's biological sample; and (iii) sequencing the extracted nucleic acid. In an aspect, sequencing the extracted nucleic acid can generate sequence data. In an aspect, characterizing the microbiome can further comprise analyzing the sequence data using taxonomic classification. In an aspect, using taxonomic classification can comprise PCR amplification. In an aspect, PCR amplification can comprise using primers targeting the 16S rRNA gene. In an aspect, PCR amplification can comprise primers targeting the V4 variable region of the 16S rRNA gene. In an aspect, disclosed primers can comprise a pair of primers. In an aspect, a disclosed pair of primers can comprise a primer having the sequence of SEQ ID NO:01 and a primer having the sequence of SEQ ID NO:02. In an aspect, a disclosed pair of primers can comprise a primer having the sequence of SEQ ID NO:03 and a primer having the sequence of SEQ ID NO:04. In an aspect, a disclosed pair of primers can comprise a primer having the sequence of SEQ ID NO:05 and a primer having the sequence of SEQ ID NO:06.

In an aspect, a disclosed method of treating S. pneumoniae colonization and/or infection can further comprise identifying the one or more pathogenic bacteria. In an aspect, the disclosed pathogenic bacteria can comprise Streptococcus sp. In an aspect, Streptococcus sp. can comprise S. pneumoniae. In an aspect, the disclosed pathogenic bacteria can comprise Staphylococcus sp. In an aspect, Staphylococcus sp. can comprise methicillin-sensitive S. aureus or methicillin-resistant S. aureus. In an aspect, the disclosed pathogenic bacteria can comprise Haemophilus influenzae or Moraxella catarrhalis.

In an aspect, a disclosed method of treating S. pneumoniae colonization and/or infection can further comprise diagnosing the subject with an infection. In an aspect, a diagnosed infection can comprise a respiratory infection. In an aspect, a diagnosed infection can affect the subject's mouth, nose, ears, sinuses, pharynx, trachea, bronchial tubes, lungs, alveoli, bronchioles, capillaries, lung lobes, pleura, cilia, epiglottis, larynx, or any combination thereof.

In an aspect, a disclosed method of treating S. pneumoniae colonization and/or infection can further comprise diagnosing the subject with pneumonia, otitis media, sinusitis, rhinosinusitis, or any combination thereof. In an aspect, otitis media (OM) can be considered a general term for middle-ear inflammation and can be classified clinically as acute otitis media (AOM), recurrent AOM, OM with effusion (OME), or chronic OME, chronic suppurative OM (CSOM), or middle ear effusion (MEE) (discussed supra).

In an aspect, sinusitis can occur when the tissue lining of the sinuses becomes swollen or inflamed. Sinusitis can comprise acute sinusitis (e.g., when symptoms are present for 4 weeks or less), chronic sinusitis (e.g., when the swelling of the sinuses is present for longer than 3 months), or subacute sinusitis (e.g., when the swelling is present between one and three months).

In an aspect, rhinosinusitis can occur when the mucosal lining in the paranasal sinuses and nasal cavity becomes inflamed. Most cases of acute rhinosinusitis are caused by viral upper respiratory infections.

In an aspect, a disclosed biotherapeutic can comprise a probiotic, a consortium of probiotics, factors secreted from a probiotic, factors secreted from a consortium of probiotics, or any combination thereof.

In an aspect, a disclosed biotherapeutic can comprise pharmaceutical formulation comprising a probiotic, a consortium of probiotics, factors secreted from a probiotic, factors secreted from a consortium of probiotics, or any combination thereof, and a pharmaceutically acceptable carrier and/or excipient. In an aspect, a disclosed pharmaceutical formulation can comprise at least one lyoprotectant. A disclosed lyoprotectant can comprise peptone, glycerol, lactose, gelatin, glucose, sucrose, trehalose, dextran, maltodextrin, adonitol, sodium glutamate, or any combination thereof.

In an aspect, a disclosed biotherapeutic can be lyophilized or freeze-dried such that, for example, a disclosed probiotic, a disclosed consortium of probiotics, disclosed factors secreted from a probiotic, disclosed factors secreted from a consortium of probiotics, or any combination thereof are all lyophilized or freeze-dried.

In an aspect, a disclosed consortium of probiotics can comprise at least one strain from the bacterial genus Corynebacterium, at least one strain from the bacterial genus Dolosigranulum, at least one strain from the bacterial genus Streptococcus, and at least one strain from the bacterial genus Lactobacillus.

In an aspect, the at least one strain from the bacterial genus Corynebacterium can comprise one or more strains of Corynebacterium accolens. In an aspect, the at least one strain from the bacterial genus Dolosigranulum can comprise one or more strains of Dolosigranulum pigrum. In an aspect, the at least one strain from the bacterial genus Streptococcus can comprise one or more strains of Streptococcus mitis, Streptococcus salivarius, Streptococcus oralis, or Streptococcus thermophilus. In an aspect, the at least one strain from the bacterial genus Lactobacillus can comprise one or more strains of Lactobacillus rhamnosus or Lactobacillus acidophilus.

In an aspect of a disclosed method of treating S. pneumoniae colonization and/or infection, a disclosed biotherapeutic can comprises one or more probiotics.

In an aspect, one or more disclosed probiotics can comprise at least one strain from the bacterial genus one Corynebacterium sp. In an aspect of a disclosed pharmaceutical formulation, one or more disclosed probiotics can comprise at least one strain from the bacterial genus Corynebacterium and at least one strain from the bacterial genus Dolosigranulum. In an aspect of a disclosed pharmaceutical formulation, one or more disclosed probiotics can comprise at least one strain from the bacterial genus Corynebacterium, at least one strain from the bacterial genus Dolosigranulum, and at least one strain from the bacterial genus Streptococcus. In an aspect of a disclosed pharmaceutical formulation, one or more disclosed probiotics can comprise at least one strain from the bacterial genus Corynebacterium, at least one strain from the bacterial genus Dolosigranulum, and at least one strain from the bacterial genus Lactobacillus. In an aspect of a disclosed pharmaceutical formulation, one or more disclosed probiotics can comprise at least one strain from the bacterial genus Corynebacterium and at least one at least one strain from the bacterial genus Streptococcus. In an aspect of a disclosed pharmaceutical formulation, one or more disclosed probiotics can comprise at least one strain from the bacterial genus Corynebacterium, at least one strain from the bacterial genus Streptococcus, and at least one strain from the bacterial genus Lactobacillus. In an aspect of a disclosed pharmaceutical formulation, one or more probiotics can comprise at least one strain from the bacterial genus Corynebacterium and at least one strain from the bacterial genus Lactobacillus. In an aspect of a disclosed pharmaceutical formulation, one or more probiotics can comprise at least one strain from the bacterial genus Corynebacterium, at least one strain from the bacterial genus Dolosigranulum, at least one strain from the bacterial genus Streptococcus, and at least one strain from the bacterial genus Lactobacillus.

In an aspect of a disclosed method of treating S. pneumoniae colonization and/or infection, a disclosed biotherapeutic can comprise one or more factors secreted by a disclosed probiotic or secreted by a disclosed consortium of probiotics. In an aspect of a disclosed pharmaceutical formulation, a disclosed biotherapeutic can comprise one or more factors secreted by at least one strain from the bacterial genus Corynebacterium, at least one strain from the bacterial genus Dolosigranulum, at least one strain from the bacterial genus Streptococcus, and at least one strain from the bacterial genus Lactobacillus. In an aspect of a disclosed pharmaceutical formulation, a disclosed biotherapeutic can comprise one or more factors secreted by at least one strain from the bacterial genus Corynebacterium. In an aspect of a disclosed pharmaceutical formulation, a disclosed biotherapeutic can comprise one or more factors secreted by at least one strain from the bacterial genus Corynebacterium and at least one strain from the bacterial genus Dolosigranulum. In an aspect of a disclosed pharmaceutical formulation, a disclosed biotherapeutic can comprise one or more factors secreted by at least one strain from the bacterial genus Corynebacterium, at least one strain from the bacterial genus Dolosigranulum, and at least one train from the bacterial genus Streptococcus. In an aspect of a disclosed pharmaceutical formulation, a disclosed biotherapeutic can comprise one or more factors secreted by at least one strain from the bacterial genus Corynebacterium, at least one strain from the bacterial genus Dolosigranulum, and at least one strain from the bacterial genus Lactobacillus. In an aspect of a disclosed pharmaceutical formulation, a disclosed biotherapeutic can comprise one or more factors secreted by at least one strain from the bacterial genus Corynebacterium and at least one strain from the bacterial genus Streptococcus. In an aspect of a disclosed pharmaceutical formulation, a disclosed biotherapeutic can comprise comprises one or more factors secreted by at least one strain from the bacterial genus Corynebacterium, at least one strain from the bacterial genus Streptococcus, and at least one strain from the bacterial genus Lactobacillus. In an aspect of a disclosed pharmaceutical formulation, a disclosed biotherapeutic can comprise one or more factors secreted by at least one strain from the bacterial genus Corynebacterium and at least one strain from the bacterial genus Lactobacillus.

In an aspect, a disclosed Corynebacterium can comprise C. accolens, C. afermentans subsp. afermentans, C. afermentans subsp. lipophilum, C. ammoniagenes, C. amycolatum, C. appendicis, C. aquaticum, C. argentoratense, C. atypicum, C. aurimucosum, C. auris, C. bovis, C. canis, C. confusum, C. coyleae, C. diphtheriae, C. durum, C. efficiens, C. equi (now Rhodococcus equi), C. falsenii, C. flavescens, C. freiburgense, C. freneyi, C. glucuronolyticum, C. glutamicum, C. granulosum, C. haemolyticum, C. halofytica, C. kroppenstedtii, C. hansenii, C. imitans, C. jeikeium (group JK), C. kroppenstedtii, C. kutscheri, C. lipophiloflavum, C. macginleyi, C. massiliense, C. mastitidis-like, C. matruchotii, C. minutissimum, C. mucifaciens, C. mycetoides, C. ovis, C. parvum (Propionibacterium acnes), C. paurometabolum, C. pilbarense, C. propinquum, C. pseudodiphtheriticum (C. hofmannii), C. pseudotuberculosis, C. pyogenes-Trueperella pyogenes, C. pyruviciproducens, C. renale, C. resistans, C. riegelii, C. simulans, C. singular, C. spec, C. sputi, C. stationis, C. striatum, C. sundsvallense, C. tenuis, C. thomsenii, C. timonense, C. tuberculostearicum, C. tuscaniense, C. ulcerans, C. urealyticum (group D2), C. urealyticum, C. xerosis, Corynebacterium_BWA136, Corynebacterium_BWA297, Corynebacterium_DU041, Corynebacterium_DU044, or any combination thereof. In an aspect of a disclosed pharmaceutical formulation, a disclosed strain from the bacterial genus Corynebacterium can comprise C. accolens, C. coyleae, C. propinquum, C. pseudodiphtheriticum, C. amycolatum, C. glutamicum, C. aurimucosum, C. tuberculostearicum, C. afermentans, C. striatum, Corynebacterium_DU041, Corynebacterium_DU044, Corynebacterium_BWA136, Corynebacterium_BWA297, or any combination thereof. In an aspect, a disclosed strain from the bacterial genus Corynebacterium can comprise any species identified in Table 13.

In an aspect of a disclosed method of treating S. pneumoniae colonization and/or infection, a disclosed therapeutically effective dose can comprise a total of at least 100 bacterial CFUs of the one or more probiotics, a total of at least 200 to at least 300 bacterial CFUs of the one or more probiotics, a total of at least 300 to at least 400 bacterial CFUs of the one or more probiotics, a total of at least 400 to at least 500 bacterial CFUs of the one or more probiotics, a total of at least 500 to at least 600 bacterial CFUs of the one or more probiotics, a total of at least 600 to at least 700 bacterial CFUs of the one or more probiotics, a total of at least 700 to at least 800 bacterial CFUs of the one or more probiotics, a total of at least 800 to at least 900 bacterial CFUs of the one or more probiotics, a total of at least 900 to at least 1000 bacterial CFUs of the one or more probiotics, or a total of at least 1000 bacterial CFUs of one or more disclosed probiotics (such as, for example, Corynebacterium sp., Dolosigranulum sp., Streptococcus sp., Lactobacillus sp., or any combination thereof).

In an aspect of a disclosed method of treating S. pneumoniae colonization and/or infection, a disclosed therapeutically effective dose can comprise a total of at least 1000 to at least 2000 bacterial CFUs of the one or more probiotics, a total of at least 2000 to at least 3000 bacterial CFUs of the one or more probiotics, a total of at least 3000 to at least 4000 bacterial CFUs of the one or more probiotics, a total of at least 4000 to at least 5000 bacterial CFUs of the one or more probiotics, a total of at least 5000 to at least 6000 bacterial CFUs of the one or more probiotics, a total of at least 6000 to at least 7000 bacterial CFUs of the one or more probiotics, a total of at least 8000 to at least 9000 bacterial CFUs of the one or more probiotics, or a total of at least 10,000 bacterial CFUs of one or more disclosed probiotics (such as, for example, Corynebacterium sp., Dolosigranulum sp., Streptococcus sp., Lactobacillus sp., or any combination thereof).

In an aspect of a disclosed method of treating S. pneumoniae colonization and/or infection, a disclosed therapeutically effective dose can comprise a total of at least 100 bacterial CFUs of Corynebacterium sp., Dolosigranulum sp., Streptococcus sp., Lactobacillus sp., or any combination thereof, a total of at least 200 to at least 300 bacterial CFUs of Corynebacterium sp., Dolosigranulum sp., Streptococcus sp., Lactobacillus sp., or any combination thereof, a total of at least 300 to at least 400 bacterial CFUs of Corynebacterium sp., Dolosigranulum sp., Streptococcus sp., Lactobacillus sp., or any combination thereof, a total of at least 400 to at least 500 bacterial CFUs of Corynebacterium sp., Dolosigranulum sp., Streptococcus sp., Lactobacillus sp., or any combination thereof, a total of at least 500 to at least 600 bacterial CFUs of Corynebacterium sp., Dolosigranulum sp., Streptococcus sp., Lactobacillus sp., or any combination thereof, a total of at least 600 to at least 700 bacterial CFUs of Corynebacterium sp., Dolosigranulum sp., Streptococcus sp., Lactobacillus sp., or any combination thereof, a total of at least 700 to at least 800 bacterial CFUs of Corynebacterium sp., Dolosigranulum sp., Streptococcus sp., Lactobacillus sp., or any combination thereof, a total of at least 800 to at least 900 bacterial CFUs of Corynebacterium sp., Dolosigranulum sp., Streptococcus sp., Lactobacillus sp., or any combination thereof, a total of at least 900 to at least 1000 bacterial CFUs of Corynebacterium sp., Dolosigranulum sp., Streptococcus sp., Lactobacillus sp., or any combination thereof, or a total of at least 1000 bacterial CFUs of Corynebacterium sp., Dolosigranulum sp., Streptococcus sp., Lactobacillus sp., or any combination thereof.

In an aspect of a disclosed method of treating S. pneumoniae colonization and/or infection, a disclosed therapeutically effective dose can comprise a total of at least 1000 to at least 2000 bacterial CFUs of Corynebacterium sp., Dolosigranulum sp., Streptococcus sp., Lactobacillus sp., or any combination thereof, a total of at least 2000 to at least 3000 bacterial CFUs of Corynebacterium sp., Dolosigranulum sp., Streptococcus sp., Lactobacillus sp., or any combination thereof, a total of at least 3000 to at least 4000 bacterial CFUs of Corynebacterium sp., Dolosigranulum sp., Streptococcus sp., Lactobacillus sp., or any combination thereof, a total of at least 4000 to at least 5000 bacterial CFUs of the Corynebacterium sp., Dolosigranulum sp., Streptococcus sp., Lactobacillus sp., or any combination thereof, a total of at least 5000 to at least 6000 bacterial CFUs of Corynebacterium sp., Dolosigranulum sp., Streptococcus sp., Lactobacillus sp., or any combination thereof, a total of at least 6000 to at least 7000 bacterial CFUs of Corynebacterium sp., Dolosigranulum sp., Streptococcus sp., Lactobacillus sp., or any combination thereof, a total of at least 8000 to at least 9000 bacterial CFUs of Corynebacterium sp., Dolosigranulum sp., Streptococcus sp., Lactobacillus sp., or any combination thereof, or a total of at least 10,000 bacterial CFUs of Corynebacterium sp., Dolosigranulum sp., Streptococcus sp., Lactobacillus sp., or any combination thereof.

In an aspect, a disclosed therapeutically effective dose can comprise a total of at least 10⁴ to at least 10⁵ bacterial CFUs of the one or more probiotics, a total of at least 10⁵ to at least 10⁶ bacterial CFUs of the one or more probiotics, a total of at least 10⁶ to at least 10⁷ bacterial CFUs of the one or more probiotics, a total of at least 10⁷ to at least 10⁸ bacterial CFUs of the one or more probiotics, a total of at least 10⁸ to at least 10⁹ bacterial CFUs of the one or more probiotics, a total of at least 10⁹ to at least 10¹⁰ bacterial CFUs of the one or more probiotics, a total of at least 10¹⁰ to at least 10¹¹ bacterial CFUs of the one or more probiotics, a total of at least 10¹¹ to at least 10¹² bacterial CFUs of the one or more probiotics, a total of at least 10¹² bacterial CFUs of the one or more probiotics, or more than 10¹² bacterial CFUs of one or more disclosed probiotics (such as, for example, at least one strain from the bacterial genus Corynebacterium, at least one strain from the bacterial genus Dolosigranulum, at least one strain from the bacterial genus Streptococcus, and at least one strain from the bacterial genus Lactobacillus, or any combination thereof).

In an aspect, a disclosed therapeutically effective dose can comprise at least 10³, 10⁴, 10⁵, 10⁶, 10⁷, 10⁸, 10⁹, 10¹⁰, 10¹¹, or 10¹² CFUs of a disclosed probiotic or a consortium of disclosed probiotics, preferably at least 1.2×10³, 1.4×10³, 2×10³, or 3×10³ CFUs of a disclosed probiotic or a consortium of disclosed probiotics, at least 5×10³, 5.02×10³, 5.04×10³, 5.2×10³, or 5.4×10³ CFUs of a disclosed probiotic or a consortium of disclosed probiotics, at least 1.02×10⁴, 1.04×10⁴, 1.2×10⁴, 1.4×10⁴, 1.5×10⁴, 2×10⁴, or 3×10⁴ CFUs of a disclosed probiotic or a consortium of disclosed probiotics, at least 5×10⁴, 5.02×10⁴, 5.04×10⁴, 5.2×10⁴, or 5.4×10⁴ CFUs of a disclosed probiotic or a consortium of disclosed probiotics, at least 1.02×10⁵, 1.04×10⁵, 1.2×10⁵, 1.4×10⁵, 1.5×10⁵, 2×10⁵, or 3×10⁵ CFUs of a disclosed probiotic or a consortium of disclosed probiotics, at least 1.02×10⁶, 1.04×10⁶, 1.2×10⁶, 1.4×10⁶, 2×10⁶, or 3×10⁶ CFUs of a disclosed probiotic or a consortium of disclosed probiotics, at least 1.02×10⁷, 1.04×10⁷, 1.2×10⁷, 1.4×10⁷, 2×10⁷, or 3×10⁷ CFUs of a disclosed probiotic or a consortium of disclosed probiotics, at least 1.02×10⁸, 1.04×10⁸, 1.2×10⁸, 1.4×10⁸, 2×10⁸, or 3×10⁸ CFUs of a disclosed probiotic or a consortium of disclosed probiotics, at least 1.02×10⁹, 1.04×10⁹, 1.2×10⁹, 1.4×10⁹, 2×10⁹, or 3×10⁹ CFUs of a disclosed probiotic or a consortium of disclosed probiotics, at least 10¹⁰ CFUs of a disclosed probiotic or a consortium of disclosed probiotics, at least 1.02×10¹⁰, 1.04×10¹⁰, 1.2×10¹⁰, 1.4×10¹⁰, 2×10¹⁰, or 3×10¹⁰ CFUs of a disclosed probiotic or a consortium of disclosed probiotics, at least 1.02×10¹¹, 1.04×10¹¹, 1.2×10¹¹, 1.4×10¹¹, 2×10¹¹, or 3×10¹¹ CFUs of a disclosed probiotic or a consortium of disclosed probiotics, or at least 1.02×10¹², 1.04×10¹², 1.2×10¹², 1.4×10¹², 2×10¹², or 3×10¹² CFUs of a disclosed probiotic or a consortium of disclosed probiotics per dose.

In an aspect, a disclosed therapeutically effective dose can comprise any amount or an unlimited amount of bacterial CFUs of the one or more probiotics.

In an aspect of a disclosed method of treating S. pneumoniae colonization and/or infection, a disclosed therapeutically effective dose can comprise Corynebacterium sp., Dolosigranulum sp., Streptococcus sp., and Lactobacillus sp. in a ratio of about 1:0.01:0.01:0.01 to about 1:1:1:1. In an aspect, a disclosed therapeutically effective dose can comprise Corynebacterium sp., Dolosigranulum sp., Streptococcus sp., and Lactobacillus sp. in any ratio. In an aspect, a disclosed therapeutically effective dose can comprise a higher dose of Corynebacterium sp., or a higher dose of Dolosigranulum sp., or a higher dose of Streptococcus sp., or a higher dose of Lactobacillus sp. as compared to any other bacteria in the consortium. In an aspect, a disclosed therapeutically effective dose can comprise a dose of Lactobacillus sp. that is about double that of Corynebacterium sp.

In an aspect, a disclosed method of treating S. pneumoniae colonization and/or infection can further comprise treating the subject. In an aspect, treating the subject can comprise treating the subject's respiratory infection. In an aspect, treating the subject can comprise treating the subject's non-respiratory infection.

In an aspect, a disclosed method of treating S. pneumoniae colonization and/or infection can comprise further comprising administering to the subject a therapeutically effective amount of one or more anti-bacterial agents. Anti-bacterial agents and combinations of anti-bacterial agents are known to the art and discussed supra in Part VII(C)(1).

In an aspect, a disclosed method of treating S. pneumoniae colonization and/or infection can comprise further comprising administering to the subject a therapeutically effective amount of (i) one or more active agents, (ii) one or more biologically active agents, (iii) one or more pharmaceutically active agents, (iv) one or more immune-based therapeutic agents, (v) one or more clinically approved agents, or (vi) any combination thereof. In an aspect, a therapeutically effective amount of an active agent, a biologically active agent, a pharmaceutically active agent, an immune-based therapeutic active agent, a clinically approved agent, or an anti-bacterial agent can be about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, administering a disclosed anti-bacterial agent can comprise systemic or direct administration. In an aspect, administering a disclosed anti-bacterial agent can comprise oral administration, sublingual administration, intravenous administration, intranasal administration, intraperitoneal administration, or any combination thereof. In an aspect, administering a disclosed anti-bacterial agent can be administered by any method of administration disclosed herein. In an aspect, a disclosed anti-bacterial agent can be administered via multiple routes either concurrently or sequentially. A skilled clinician can determine the best route of administration for a disclosed anti-bacterial agent to a subject at a given time. In an aspect, a disclosed anti-bacterial agent can inherently encompass a pharmaceutically acceptable salt thereof.

In an aspect, a disclosed method of treating S. pneumoniae colonization and/or infection can further comprise increasing the relative abundance of one or more probiotics in the subject's microbiome. In an aspect, a disclosed method of treating S. pneumoniae colonization and/or infection can further comprise increasing the relative abundance of one or more of Corynebacterium sp., Dolosigranulum sp., Streptococcus sp., and Lactobacillus sp. in the subject's microbiome. In an aspect, a disclosed method of treating S. pneumoniae colonization and/or infection can further comprise increasing the relative abundance of one or more of at least one strain from the bacterial genus Corynebacterium, at least one strain from the bacterial genus Dolosigranulum, at least one strain from the bacterial genus Streptococcus, and at least one strain from the bacterial genus Lactobacillus, or any combination thereof in the subject's microbiome. In an aspect, a disclosed method of treating S. pneumoniae colonization and/or infection can further comprise increasing the relative abundance of at least one strain from the bacterial genus Corynebacterium in the subject's microbiome. In an aspect, a disclosed method of treating S. pneumoniae colonization and/or infection can further comprise increasing the relative abundance of at least one strain from the bacterial genus Dolosigranulum in the subject's microbiome. In an aspect, a disclosed method of treating S. pneumoniae colonization and/or infection can further comprise increasing the relative abundance of at least one strain from the bacterial genus Streptococcus in the subject's microbiome. In an aspect, a disclosed method of treating S. pneumoniae colonization and/or infection can further comprise increasing the relative abundance of at least one strain from the bacterial genus Lactobacillus in the subject's microbiome.

In an aspect, a disclosed method of treating S. pneumoniae colonization and/or infection can further comprise decreasing the relative abundance of one or more pathogenic bacteria in the subject's microbiome. In an aspect, a disclosed method of treating S. pneumoniae colonization and/or infection can further comprise decreasing the relative abundance of S. pneumoniae in the subject's microbiome. In an aspect, a disclosed method of treating S. pneumoniae colonization and/or infection can further comprise decreasing the relative abundance of a S. aureus in the subject's microbiome. In an aspect, a disclosed method of treating S. pneumoniae colonization and/or infection can further comprise decreasing the relative abundance of Haemophilus influenzae and/or Moraxella catarrhalis in the subject's microbiome.

In an aspect, a disclosed method of treating S. pneumoniae colonization and/or infection can further comprise administering to the subject one or more therapeutic agents. In an aspect, a disclosed therapeutic agent can comprise a biologically active agent, a pharmaceutically active agent, an anti-bacterial agent, an anti-fungal agent, an anti-viral agent, a corticosteroid, an analgesic, an immunostimulant, an immune-based product, or any combination thereof. In an aspect of a disclosed method of promoting respiratory health, administering can comprise intranasal administration, oral administration, sublingual administration, or any combination thereof.

In an aspect of a disclosed method of treating S. pneumoniae colonization and/or infection, a subject can be a healthy subject, for example, a subject that does not have an infection and/or is not suspected of having an infection.

In an aspect, a subject can be an adult, a child, or an infant. In an aspect, a subject can be a neonate. In an aspect, a subject can be a premature infant. In an aspect, a subject can be immune-compromised. In an aspect, a subject can have diabetes or a chronic disease (e.g., heart disease, kidney disease, or liver disease). In an aspect, a subject can have HIV. In an aspect, a subject can have cancer or has had cancer. In an aspect, a subject can be the recipient of one or more solid organ transplants. In an aspect, a subject can have nephrotic syndrome. In an aspect, a subject can be alcoholic or can smoke cigarettes. In an aspect, a subject can have a chronic lung disease. In an aspect, a disclosed chronic lung disease can comprise emphysema, chronic obstructive lung disease, chronic obstructive pulmonary disease, asthma, or any combination thereof.

In an aspect, a subject can be diagnosed with an infection of one or more parts of the respiratory system. In an aspect, a subject can be suspected of having an infection in one or more parts of the respiratory system. In an aspect, a disclosed therapeutically effective amount of a biotherapeutic can comprise an amount sufficient to inhibit and/or prevent the growth of one or more pathogenic bacteria in one or more parts of the subject's respiratory system. In an aspect, a disclosed therapeutically effective amount of a biotherapeutic can comprise an amount sufficient to inhibit and/or prevent the spread of one or more pathogenic bacteria from a first part of the subject's respiratory system to a second part of the subject's respiratory system.

In an aspect, one or more disclosed probiotics can inhibit and/or prevent the growth and/or spread of one or more pathogenic bacteria using competitive adherence and/or non-adherence mechanisms (e.g., secretion of antimicrobial factors). In an aspect, one or more disclosed probiotics can inhibit and/or prevent the adherence and/or colonization of one or more pathogenic bacteria. In an aspect, one or more disclosed probiotics can modulate the endocytosis or paracellular migration of one or more pathogenic bacteria. In an aspect, one or more disclosed probiotics can inhibit and/or prevent the spread of one or more pathogenic bacteria by competitive adherence and/or niche occupancy.

In an aspect, one or more disclosed probiotics can inhibit and/or prevent the growth and/or spread of S. pneumoniae using competitive adherence and/or non-adherence mechanisms (e.g., secretion of antimicrobial factors). In an aspect, one or more disclosed probiotics can inhibit and/or prevent the adherence and/or colonization of S. pneumoniae. In an aspect, one or more disclosed probiotics can modulate the endocytosis or paracellular migration of S. pneumoniae. In an aspect, one or more disclosed probiotics can inhibit and/or prevent the spread S. pneumoniae by competitive adherence and/or niche occupancy.

In an aspect of a disclosed method of treating S. pneumoniae colonization and/or infection, one or more disclosed probiotics can secrete one or more factors. In an aspect, a disclosed secreted factor can be species-dependent and/or strain-dependent. In an aspect, a disclosed secreted factor can inhibit and/or prevent the growth and/or spread of one or more pathogenic bacteria. In an aspect, a disclosed secreted factor can inhibit and/or prevent the adherence and/or colonization of one or more pathogenic bacteria. In an aspect, a disclosed secreted factor can modulate the endocytosis or paracellular migration of one or more pathogenic bacteria. In an aspect, a disclosed secreted factor can inhibit and/or prevent the spread of one or more pathogenic bacteria by competitive adherence and/or niche occupancy.

In an aspect of a disclosed method of treating S. pneumoniae colonization and/or infection, a disclosed secreted factor can inhibit and/or prevent the growth and/or spread of S. pneumoniae.

In an aspect, a disclosed secreted factor can inhibit and/or prevent the adherence and/or colonization of S. pneumoniae. In an aspect, a disclosed secreted factor can modulate the endocytosis or paracellular migration of S. pneumoniae. In an aspect, a disclosed secreted factor can inhibit and/or prevent the spread of S. pneumoniae by competitive adherence and/or niche occupancy.

In an aspect of a disclosed method of treating S. pneumoniae colonization and/or infection, Corynebacterium sp., Dolosigranulum sp., Streptococcus sp., Lactobacillus sp., or any combination thereof can secrete one or more factors that inhibit and/or prevent the growth and/or spread of one or more pathogenic bacteria. In an aspect of a disclosed method of treating S. pneumoniae colonization and/or infection, Corynebacterium sp., Dolosigranulum sp., Streptococcus sp., Lactobacillus sp., or any combination thereof can secrete one or more factors that inhibit and/or prevent the adherence and/or colonization of one or more pathogenic bacteria. In an aspect of a disclosed method of treating S. pneumoniae colonization and/or infection, Corynebacterium sp., Dolosigranulum sp., Streptococcus sp., Lactobacillus sp., or any combination thereof can secrete one or more secreted factors that modulate the endocytosis or paracellular migration of one or more pathogenic bacteria. In an aspect of a disclosed method of treating S. pneumoniae colonization and/or infection, Corynebacterium sp., Dolosigranulum sp., Streptococcus sp., Lactobacillus sp., or any combination thereof can secrete one or more secreted factors inhibit and/or prevent the spread of one or more pathogenic bacteria by competitive adherence and/or niche occupancy. In an aspect of a disclosed method of treating S. pneumoniae colonization and/or infection, Corynebacterium sp., Dolosigranulum sp., Streptococcus sp., Lactobacillus sp., or any combination thereof can secrete one or more secreted factors inhibit and/or prevent the growth and/or spread of S. pneumoniae. In an aspect of a disclosed method of treating S. pneumoniae colonization and/or infection, Corynebacterium sp., Dolosigranulum sp., Streptococcus sp., Lactobacillus sp., or any combination thereof can secrete one or more factors inhibit and/or prevent the adherence and/or colonization of S. pneumoniae. In an aspect, a disclosed method of treating S. pneumoniae colonization and/or infection, Corynebacterium sp., Dolosigranulum sp., Streptococcus sp., Lactobacillus sp., or any combination thereof secrete one or more factors can modulate the endocytosis or paracellular migration of S. pneumoniae. In an aspect of a disclosed method of treating S. pneumoniae colonization and/or infection, Corynebacterium sp., Dolosigranulum sp., Streptococcus sp., Lactobacillus sp., or any combination thereof can secrete one or more factors inhibit and/or prevent the spread of S. pneumoniae by competitive adherence and/or niche occupancy.

In an aspect of a disclosed method of treating S. pneumoniae colonization and/or infection, Corynebacterium sp. can secrete one or more factors that inhibit and/or prevent the growth and/or spread of one or more pathogenic bacteria. In an aspect of a disclosed method of treating S. pneumoniae colonization and/or infection, Corynebacterium sp. can secrete one or more factors that inhibit and/or prevent the adherence and/or colonization of one or more pathogenic bacteria.

In an aspect of a disclosed method of treating S. pneumoniae colonization and/or infection, Corynebacterium sp. can secrete one or more secreted factors that modulate the endocytosis or paracellular migration of one or more pathogenic bacteria. In an aspect of a disclosed method of treating S. pneumoniae colonization and/or infection, Corynebacterium sp. can secrete one or more secreted factors that inhibit and/or prevent the spread of one or more pathogenic bacteria by competitive adherence and/or niche occupancy.

In an aspect of a disclosed method of treating S. pneumoniae colonization and/or infection, Corynebacterium sp. can secrete one or more secreted factors that inhibit and/or prevent the growth and/or spread of S. pneumoniae. In an aspect of a disclosed method of treating S. pneumoniae colonization and/or infection, Corynebacterium sp. can secrete one or more factors that inhibit and/or prevent the adherence and/or colonization of S. pneumoniae. In an aspect of a disclosed method of treating S. pneumoniae colonization and/or infection, Corynebacterium sp. can secrete one or more factors that modulate the endocytosis or paracellular migration of S. pneumoniae. In an aspect of a disclosed method of treating S. pneumoniae colonization and/or infection, Corynebacterium sp. can secrete one or more factors that inhibit and/or prevent the spread of S. pneumoniae by competitive adherence and/or niche occupancy.

In an aspect, a disclosed method of treating S. pneumoniae colonization and/or infection can comprise a 10%, 20%, 30%, 40%, 50%, 60%, 70%, 80%, 90%, 100%, or any amount of inhibiting and/or preventing the growth and/or spread of one or more pathogenic bacteria (e.g., S. pneumoniae) when compared to a control subject (such as, for example, a subject that has not received a disclosed biotherapeutic). In an aspect, a disclosed method of treating S. pneumoniae colonization and/or infection can comprise a 10-20%, 20-30%, 30-40%, 40-50%, 50-60%, 60-70%, 70-80%, 80-90%, or 90-100% or any amount of inhibiting and/or preventing the growth and/or spread of one or more pathogenic bacteria (e.g., S. pneumoniae) when compared to a control subject (such as, for example, a subject that has not received a disclosed biotherapeutic).

In an aspect, a disclosed method of treating S. pneumoniae colonization and/or infection can comprise a 10%, 20%, 30%, 40%, 50%, 60%, 70%, 80%, 90%, 100%, or any amount of inhibiting and/or preventing the adherence and/or colonization of one or more pathogenic bacteria (e.g., S. pneumoniae) when compared to a control subject (such as, for example, a subject that has not received a disclosed biotherapeutic). In an aspect, a disclosed method of treating S. pneumoniae colonization and/or infection can comprise a 10-20%, 20-30%, 30-40%, 40-50%, 50-60%, 60-70%, 70-80%, 80-90%, or 90-100% or any amount of inhibiting and/or preventing the adherence and/or colonization of one or more pathogenic bacteria (e.g., S. pneumoniae) when compared to a control subject (such as, for example, a subject that has not received a disclosed biotherapeutic).

In an aspect, a disclosed method of treating S. pneumoniae colonization and/or infection can comprise a 10%, 20%, 30%, 40%, 50%, 60%, 70%, 80%, 90%, 100%, or any amount of modulating the endocytosis or paracellular migration of one or more pathogenic bacteria (e.g., S. pneumoniae) when compared to a control subject (such as, for example, a subject that has not received a disclosed biotherapeutic). In an aspect, a disclosed method of treating S. pneumoniae colonization and/or infection can comprise a 10-20%, 20-30%, 30-40%, 40-50%, 50-60%, 60-70%, 70-80%, 80-90%, or 90-100% or any amount of modulating the endocytosis or paracellular migration of one or more pathogenic bacteria (e.g., S. pneumoniae) when compared to a control subject (such as, for example, a subject that has not received a disclosed biotherapeutic).

In an aspect, a disclosed method of treating S. pneumoniae colonization and/or infection can comprise a 10%, 20%, 30%, 40%, 50%, 60%, 70%, 80%, 90%, 100%, or any amount of inhibiting and/or preventing the spread of one or more pathogenic bacteria (e.g., S. pneumoniae) by competitive adherence and/or niche occupancy when compared to a control subject (such as, for example, a subject that has not received a disclosed biotherapeutic). In an aspect, a disclosed method of treating S. pneumoniae colonization and/or infection can comprise a 10-20%, 20-30%, 30-40%, 40-50%, 50-60%, 60-70%, 70-80%, 80-90%, or 90-100% or any amount of inhibiting and/or preventing the spread of one or more pathogenic bacteria (e.g., S. pneumoniae) by competitive adherence and/or niche occupancy when compared to a control subject (such as, for example, a subject that has not received a disclosed biotherapeutic).

In an aspect, a disclosed method of treating S. pneumoniae colonization and/or infection can comprise repeating the administering of one or more disclosed biotherapeutics, one or more disclosed secreted factors, a disclosed consortium of probiotics, one or more disclosed probiotics, or any combination thereof. For example, in an aspect, repeating the administering can comprise administering one or more times daily (e.g., 1, 2, 3, or 4 times). In an aspect, repeating the administering can comprise administering on several consecutive days (e.g., for 1, 2, 3, 4, 5, 6, 7, 8, 9, or 10 days, or more than 10 days), weeks (e.g., for 1, 2, 3, 4, 5, 6, 7, 8, 9, or 10 weeks, or more than 10 weeks), or months (e.g., for 1, 2, 3, 4, 5, or 6 months, or more than 6 months). In an aspect, a disclosed biotherapeutic, a disclosed secreted factor, a disclosed consortium of probiotics, a disclosed probiotic, or any combination thereof can be administered 1-3 times per day for about 7 to 21 days.

In an aspect, a disclosed method of treating S. pneumoniae colonization and/or infection can comprise monitoring the subject. In an aspect, monitoring the subject can comprise monitoring the subject for the development of adverse effects. In an aspect, in the absence of adverse effects, a disclosed method of treating S. pneumoniae colonization and/or infection can comprise continuing to treat the subject. In an aspect, continuing to treat the subject can comprise continuing to administer to the subject one or more disclosed biotherapeutics, one or more disclosed secreted factors, a disclosed consortium of probiotics, one or more disclosed probiotics, or any combination thereof. In an aspect, in the presence of adverse effects, a disclosed method of treating S. pneumoniae colonization and/or infection can comprise modifying one or more steps of the method. In an aspect, modifying one or more steps of a disclosed method can comprise modifying the administering step. In an aspect, modifying the administering step can comprise changing the amount of one or more disclosed biotherapeutics, one or more disclosed secreted factors, a disclosed consortium of probiotics, one or more disclosed probiotics, or any combination thereof administered to the subject, changing the frequency of administration of one or more disclosed biotherapeutics, one or more disclosed secreted factors, a disclosed consortium of probiotics, one or more disclosed probiotics, or any combination thereof, changing the duration of administration of one or more disclosed biotherapeutics, one or more disclosed secreted factors, a disclosed consortium of probiotics, one or more disclosed probiotics, or any combination thereof, changing the route of administration of one or more disclosed biotherapeutics, one or more disclosed secreted factors, a disclosed consortium of probiotics, one or more disclosed probiotics, or any combination thereof, or any combination of disclosed changes.

In an aspect, a disclosed method of treating S. pneumoniae colonization and/or infection can comprise manipulating the microbiome to inhibit and/or prevent infection caused by one or more pathogenic bacteria, such as an infection caused by S. pneumoniae. In an aspect, a disclosed method of treating S. pneumoniae colonization and/or infection can comprise manipulating the microbiome to decrease and/or minimize the risk of infection caused by one or more pathogenic bacteria, such as an infection caused by S. pneumoniae. For example, in an aspect, when the relative abundance of one or more disclosed Corynebacterium sp. is below a threshold level of 1.2×10⁶ 16S rRNA gene copies per biological sample, then the risk of colonization by S. pneumoniae is increased and/or higher and/or enhanced. In an aspect, when the relative abundance of Corynebacterium sp. is below a threshold level of 1.2×10⁶ 16S rRNA gene copies per biological sample, then a disclosed method can further comprise administering and/or continuing to administer a therapeutically effective amount of a disclosed biotherapeutic. In an aspect, when the relative abundance of Corynebacterium sp. is above 1.2×10⁶ 16S rRNA gene copies per biological sample, then a disclosed method can further comprise continuing to monitor the subject.

In an aspect, a disclosed method of promoting respiratory health can comprise manipulating the microbiome to inhibit and/or prevent infection caused by one or more pathogenic bacteria, such as an infection caused by S. pneumoniae. In an aspect, a disclosed method of promoting respiratory health can comprise manipulating the microbiome to decrease and/or minimize the risk of infection caused by one or more pathogenic bacteria, such as an infection caused by S. pneumoniae. For example, in an aspect, when the relative abundance of one or more disclosed Corynebacterium sp. is below a threshold level of 1.2×10⁶ 16S rRNA gene copies per biological sample, then the risk of colonization by S. pneumoniae is increased and/or higher and/or enhanced. In an aspect, when the relative abundance of Corynebacterium sp. is below a threshold level of 1.2×10⁶ 16S rRNA gene copies per biological sample, then a disclosed method can further comprise administering and/or continuing to administer a therapeutically effective amount of a disclosed biotherapeutic. In an aspect, when the relative abundance of Corynebacterium sp. is above 1.2×10⁶ 16S rRNA gene copies per biological sample, then a disclosed method can further comprise continuing to monitor the subject.

For example, in an aspect, when the relative abundance of one or more disclosed Corynebacterium sp. is less than about 50%, about 60%, about 70%, about 80%, or 90% of the microbiome, then the risk of colonization by S. pneumoniae is increased and/or higher and/or enhanced. For example, in an aspect, when the relative abundance of one or more disclosed Corynebacterium sp. is about 50% to about 60%, about 60% to about 70%, about 70% to about 80%, about 80% to about 90% of the microbiome, then the risk of colonization by S. pneumoniae is increased and/or higher and/or enhanced. In an aspect, when the relative abundance of Corynebacterium sp. is about 50% to about 60%, about 60% to about 70%, about 70% to about 80%, about 80% to about 90% of the microbiome, then a disclosed method can further comprise administering and/or continuing to administer a therapeutically effective amount of a disclosed biotherapeutic. In an aspect, when the relative abundance of Corynebacterium sp. is less than about 50%, about 60%, about 70%, about 80%, or 90% of the microbiome, then a disclosed method can further comprise administering and/or continuing to administer a therapeutically effective amount of a disclosed biotherapeutic. In an aspect, when the relative abundance of Corynebacterium sp. is about 50% to about 60%, about 60% to about 70%, about 70% to about 80%, about 80% to about 90% of the microbiome, then a disclosed method can further comprise continuing to monitor the subject. In an aspect, when the relative abundance of Corynebacterium sp. is less than about 50%, about 60%, about 70%, about 80%, or 90%, then a disclosed method can further comprise continuing to monitor the subject.

In an aspect, a disclosed method of treating S. pneumoniae colonization and/or infection can comprise subjecting the subject to one or more invasive or non-invasive diagnostic assessments. Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect of a disclosed method of treating S. pneumoniae colonization and/or infection, techniques to monitor, measure, and/or assess the restoring one or more aspects of respiratory health can comprise qualitative (or subjective) means as well as quantitative (or objective) means. These means are known to the skilled person. For example, representative regulated variables and sensors relating to systemic homeostasis are discussed supra.

In an aspect of a disclosed method of treating S. pneumoniae colonization and/or infection, a disclosed biotherapeutic, a disclosed secreted factor, a disclosed consortium of probiotics, a disclosed probiotic, or any combination thereof can be administered in the absence of identifying the one or more pathogenic bacteria and/or in the absence of characterizing the microbiome. In an aspect of a disclosed method, a disclosed biotherapeutic, a disclosed secreted factor, a disclosed consortium of probiotics, a disclosed probiotic, or any combination thereof can be administered on demand.

In an aspect of a disclosed method of treating S. pneumoniae colonization and/or infection, a disclosed biotherapeutic can be formulated for administration as a pharmaceutical formulation.

In an aspect, a disclosed pharmaceutical formulation comprising a probiotic, a consortium of probiotics, factors secreted from a probiotic, factors secreted from a consortium of probiotics, or any combination thereof, and a pharmaceutically acceptable carrier and/or excipient can be used in any disclosed method of treating S. pneumoniae colonization and/or infection. Any pharmaceutical formulation disclosed herein can be used in a disclosed method of treating S. pneumoniae colonization and/or infection.

### G. Methods of Modulating Microbial Diversity and/or Composition

Disclosed herein is a method of modulating microbial diversity and/or composition in a subject, the method comprising characterizing the microbiome of a biological sample obtained from a subject; and administering one or more biotherapeutics to the subject, wherein following administration, the microbiome is modulated.

In an aspect of a disclosed method of modulating microbial diversity and/or composition, a subject can be any subject. In an aspect, a subject that does not have an infection and/or is not suspected of having an infection.

In an aspect, a disclosed microbiome can comprise the nasopharyngeal microbiome, the nasal microbiome, or both. In an aspect, a disclosed method of modulating microbial diversity and/or composition can further comprise reducing the subject's risk of developing a pathogenic bacterial infection. In an aspect, a disclosed method of modulating microbial diversity and/or composition can further comprise preventing and/or inhibiting the colonization of a pathogenic bacteria in the subject's respiratory system.

In an aspect, a disclosed method of modulating microbial diversity and/or composition can further comprise preventing and/or inhibiting the colonization of S. pneumoniae in the subject's respiratory system.

In an aspect of a disclosed method of modulating microbial diversity and/or composition, following administration, the colonization of a pathogenic bacteria and/or S. pneumoniae in the subject's respiratory system can be inhibited and/or prevented.

In an aspect, a disclosed method of modulating microbial diversity and/or composition can further comprise promoting respiratory health in a subject. In an aspect, a disclosed biotherapeutic can inhibit and/or prevent the growth of one or more pathogenic bacteria and/or S. pneumoniae in one or more parts of the subject's respiratory system.

In an aspect, a disclosed method of modulating microbial diversity and/or composition can further comprise obtaining a biological sample from the subject. In an aspect, a disclosed biological sample can comprise a nasal swab or lavage, a nasopharyngeal swab or lavage, or a pharyngeal swab or lavage, or any combination thereof. In an aspect, a disclosed biological sample can comprise nasal secretion and/or cough droplets.

In an aspect of a disclosed method of modulating microbial diversity and/or composition, characterizing the microbiome can comprise (i) collecting a biological sample from the subject; (ii) extracting nucleic acid from the subject's biological sample; and (iii) sequencing the extracted nucleic acid. In an aspect, sequencing the extracted nucleic acid can generate sequence data. In an aspect, characterizing the microbiome can further comprise analyzing the sequence data using taxonomic classification. In an aspect, using taxonomic classification can comprise PCR amplification. In an aspect, PCR amplification can comprise using primers targeting the 16S rRNA gene. In an aspect, PCR amplification can comprise primers targeting the V4 variable region of the 16S rRNA gene. In an aspect, disclosed primers can comprise a pair of primers. In an aspect, a disclosed pair of primers can comprise a primer having the sequence of SEQ ID NO:01 and a primer having the sequence of SEQ ID NO:02. In an aspect, a disclosed pair of primers can comprise a primer having the sequence of SEQ ID NO:03 and a primer having the sequence of SEQ ID NO:04. In an aspect, a disclosed pair of primers can comprise a primer having the sequence of SEQ ID NO:05 and a primer having the sequence of SEQ ID NO:06.

In an aspect, a disclosed method of modulating microbial diversity and/or composition can further comprise identifying the one or more pathogenic bacteria. In an aspect, the disclosed pathogenic bacteria can comprise Streptococcus sp. In an aspect, Streptococcus sp. can comprise S. pneumoniae. In an aspect, the disclosed pathogenic bacteria can comprise Staphylococcus sp. In an aspect, Staphylococcus sp. can comprise methicillin-sensitive S. aureus or methicillin-resistant S. aureus. In an aspect, the disclosed pathogenic bacteria can comprise Haemophilus influenzae or Moraxella catarrhalis.

In an aspect, a disclosed method of modulating microbial diversity and/or composition can further comprise diagnosing the subject with an infection. In an aspect, a diagnosed infection can comprise a respiratory infection. In an aspect, a diagnosed infection can affect the subject's mouth, nose, ears, sinuses, pharynx, trachea, bronchial tubes, lungs, alveoli, bronchioles, capillaries, lung lobes, pleura, cilia, epiglottis, larynx, or any combination thereof.

In an aspect, a disclosed method of modulating microbial diversity and/or composition can further comprise diagnosing the subject with pneumonia, otitis media, sinusitis, rhinosinusitis, or any combination thereof. In an aspect, otitis media (OM) can be considered a general term for middle-ear inflammation and can be classified clinically as acute otitis media (AOM), recurrent AOM, OM with effusion (OME), or chronic OME, chronic suppurative OM (CSOM), or middle ear effusion (MEE) (discussed supra).

In an aspect, sinusitis can occur when the tissue lining of the sinuses becomes swollen or inflamed. Sinusitis can comprise acute sinusitis (e.g., when symptoms are present for 4 weeks or less), chronic sinusitis (e.g., when the swelling of the sinuses is present for longer than 3 months), or subacute sinusitis (e.g., when the swelling is present between one and three months).

In an aspect, rhinosinusitis can occur when the mucosal lining in the paranasal sinuses and nasal cavity becomes inflamed. Most cases of acute rhinosinusitis are caused by viral upper respiratory infections.

In an aspect, a disclosed biotherapeutic can comprise a probiotic, a consortium of probiotics, factors secreted from a probiotic, factors secreted from a consortium of probiotics, or any combination thereof.

In an aspect, a disclosed biotherapeutic can comprise pharmaceutical formulation comprising a probiotic, a consortium of probiotics, factors secreted from a probiotic, factors secreted from a consortium of probiotics, or any combination thereof, and a pharmaceutically acceptable carrier and/or excipient. In an aspect, a disclosed pharmaceutical formulation can comprise at least one lyoprotectant. A disclosed lyoprotectant can comprise peptone, glycerol, lactose, gelatin, glucose, sucrose, trehalose, dextran, maltodextrin, adonitol, sodium glutamate, or any combination thereof.

In an aspect, a disclosed biotherapeutic can be lyophilized or freeze-dried such that, for example, a disclosed probiotic, a disclosed consortium of probiotics, disclosed factors secreted from a probiotic, disclosed factors secreted from a consortium of probiotics, or any combination thereof are all lyophilized or freeze-dried.

In an aspect, a disclosed consortium of probiotics can comprise at least one strain from the bacterial genus Corynebacterium, at least one strain from the bacterial genus Dolosigranulum, at least one strain from the bacterial genus Streptococcus, and at least one strain from the bacterial genus Lactobacillus. In an aspect of a disclosed method of promoting respiratory health, a disclosed biotherapeutic can comprises one or more probiotics.

In an aspect, the at least one strain from the bacterial genus Corynebacterium can comprise one or more strains of Corynebacterium accolens. In an aspect, the at least one strain from the bacterial genus Dolosigranulum can comprise one or more strains of Dolosigranulum pigrum. In an aspect, the at least one strain from the bacterial genus Streptococcus can comprise one or more strains of Streptococcus mitis, Streptococcus salivarius, Streptococcus oralis, or Streptococcus thermophilus. In an aspect, the at least one strain from the bacterial genus Lactobacillus can comprise one or more strains of Lactobacillus rhamnosus or Lactobacillus acidophilus.

In an aspect, one or more disclosed probiotics can comprise at least one strain from the bacterial genus one Corynebacterium sp. In an aspect of a disclosed pharmaceutical formulation, one or more disclosed probiotics can comprise at least one strain from the bacterial genus Corynebacterium and at least one strain from the bacterial genus Dolosigranulum. In an aspect of a disclosed pharmaceutical formulation, one or more disclosed probiotics can comprise at least one strain from the bacterial genus Corynebacterium, at least one strain from the bacterial genus Dolosigranulum, and at least one strain from the bacterial genus Streptococcus. In an aspect of a disclosed pharmaceutical formulation, one or more disclosed probiotics can comprise at least one strain from the bacterial genus Corynebacterium, at least one strain from the bacterial genus Dolosigranulum, and at least one strain from the bacterial genus Lactobacillus. In an aspect of a disclosed pharmaceutical formulation, one or more disclosed probiotics can comprise at least one strain from the bacterial genus Corynebacterium and at least one at least one strain from the bacterial genus Streptococcus. In an aspect of a disclosed pharmaceutical formulation, one or more disclosed probiotics can comprise at least one strain from the bacterial genus Corynebacterium, at least one strain from the bacterial genus Streptococcus, and at least one strain from the bacterial genus Lactobacillus. In an aspect of a disclosed pharmaceutical formulation, one or more probiotics can comprise at least one strain from the bacterial genus Corynebacterium and at least one strain from the bacterial genus Lactobacillus. In an aspect of a disclosed pharmaceutical formulation, one or more probiotics can comprise at least one strain from the bacterial genus Corynebacterium, at least one strain from the bacterial genus Dolosigranulum, at least one strain from the bacterial genus Streptococcus, and at least one strain from the bacterial genus Lactobacillus.

In an aspect of a disclosed method of modulating microbial diversity and/or composition, a disclosed biotherapeutic can comprise one or more factors secreted by a disclosed probiotic or secreted by a disclosed consortium of probiotics. In an aspect, a disclosed biotherapeutic can comprise one or more factors secreted by at least one strain from the bacterial genus Corynebacterium, at least one strain from the bacterial genus Dolosigranulum, at least one strain from the bacterial genus Streptococcus, and at least one strain from the bacterial genus Lactobacillus. In an aspect, a disclosed biotherapeutic can comprise one or more factors secreted by at least one strain from the bacterial genus Corynebacterium. In an aspect, a disclosed biotherapeutic can comprise one or more factors secreted by at least one strain from the bacterial genus Corynebacterium and at least one strain from the bacterial genus Dolosigranulum. In an aspect, a disclosed biotherapeutic can comprise one or more factors secreted by at least one strain from the bacterial genus Corynebacterium, at least one strain from the bacterial genus Dolosigranulum, and at least one train from the bacterial genus Streptococcus. In an aspect, a disclosed biotherapeutic can comprise one or more factors secreted by at least one strain from the bacterial genus Corynebacterium, at least one strain from the bacterial genus Dolosigranulum, and at least one strain from the bacterial genus Lactobacillus. In an aspect, a disclosed biotherapeutic can comprise one or more factors secreted by at least one strain from the bacterial genus Corynebacterium and at least one strain from the bacterial genus Streptococcus. In an aspect, a disclosed biotherapeutic can comprise comprises one or more factors secreted by at least one strain from the bacterial genus Corynebacterium, at least one strain from the bacterial genus Streptococcus, and at least one strain from the bacterial genus Lactobacillus. In an aspect, a disclosed biotherapeutic can comprise one or more factors secreted by at least one strain from the bacterial genus Corynebacterium and at least one strain from the bacterial genus Lactobacillus.

In an aspect, a disclosed Corynebacterium can comprise C. accolens, C. afermentans subsp. afermentans, C. afermentans subsp. lipophilum, C. ammoniagenes, C. amycolatum, C. appendicis, C. aquaticum, C. argentoratense, C. atypicum, C. aurimucosum, C. auris, C. bovis, C. canis, C. confusum, C. coyleae, C. diphtheriae, C. durum, C. efficiens, C. equi (now Rhodococcus equi), C. falsenii, C. flavescens, C. freiburgense, C. freneyi, C. glucuronolyticum, C. glutamicum, C. granulosum, C. haemolyticum, C. halofytica, C. kroppenstedtii, C. hansenii, C. imitans, C. jeikeium (group JK), C. kroppenstedtii, C. kutscheri, C. lipophiloflavum, C. macginleyi, C. massiliense, C. mastitidis-like, C. matruchotii, C. minutissimum, C. mucifaciens, C. mycetoides, C. ovis, C. parvum (Propionibacterium acnes), C. paurometabolum, C. pilbarense, C. propinquum, C. pseudodiphtheriticum (C. hofmannii), C. pseudotuberculosis, C. pyogenes-Trueperella pyogenes, C. pyruviciproducens, C. renale, C. resistans, C. riegelii, C. simulans, C. singular, C. spec, C. sputi, C. stationis, C. striatum, C. sundsvallense, C. tenuis, C. thomsenii, C. timonense, C. tuberculostearicum, C. tuscaniense, C. ulcerans, C. urealyticum (group D2), C. urealyticum, C. xerosis, Corynebacterium_BWA136, Corynebacterium_BWA297, Corynebacterium_DU041, Corynebacterium_DU044, or any combination thereof. In an aspect of a disclosed pharmaceutical formulation, a disclosed strain from the bacterial genus Corynebacterium can comprise C. accolens, C. coyleae, C. propinquum, C. pseudodiphtheriticum, C. amycolatum, C. glutamicum, C. aurimucosum, C. tuberculostearicum, C. afermentans, C. striatum, Corynebacterium_DU041, Corynebacterium_DU044, Corynebacterium_BWA136, Corynebacterium_BWA297, or any combination thereof. In an aspect, a disclosed strain from the bacterial genus Corynebacterium can comprise any species identified in Table 13.

In an aspect of a disclosed method of modulating microbial diversity and/or composition, a disclosed therapeutically effective dose can comprise a total of at least 100 bacterial CFUs of the one or more probiotics, a total of at least 200 to at least 300 bacterial CFUs of the one or more probiotics, a total of at least 300 to at least 400 bacterial CFUs of the one or more probiotics, a total of at least 400 to at least 500 bacterial CFUs of the one or more probiotics, a total of at least 500 to at least 600 bacterial CFUs of the one or more probiotics, a total of at least 600 to at least 700 bacterial CFUs of the one or more probiotics, a total of at least 700 to at least 800 bacterial CFUs of the one or more probiotics, a total of at least 800 to at least 900 bacterial CFUs of the one or more probiotics, a total of at least 900 to at least 1000 bacterial CFUs of the one or more probiotics, or a total of at least 1000 bacterial CFUs of one or more disclosed probiotics (such as, for example, Corynebacterium sp., Dolosigranulum sp., Streptococcus sp., Lactobacillus sp., or any combination thereof).

In an aspect of a disclosed method of modulating microbial diversity and/or composition, a disclosed therapeutically effective dose can comprise a total of at least 1000 to at least 2000 bacterial CFUs of the one or more probiotics, a total of at least 2000 to at least 3000 bacterial CFUs of the one or more probiotics, a total of at least 3000 to at least 4000 bacterial CFUs of the one or more probiotics, a total of at least 4000 to at least 5000 bacterial CFUs of the one or more probiotics, a total of at least 5000 to at least 6000 bacterial CFUs of the one or more probiotics, a total of at least 6000 to at least 7000 bacterial CFUs of the one or more probiotics, a total of at least 8000 to at least 9000 bacterial CFUs of the one or more probiotics, or a total of at least 10,000 bacterial CFUs of one or more disclosed probiotics (such as, for example, Corynebacterium sp., Dolosigranulum sp., Streptococcus sp., Lactobacillus sp., or any combination thereof).

In an aspect of a disclosed method of modulating microbial diversity and/or composition, a disclosed therapeutically effective dose can comprise a total of at least 100 bacterial CFUs of Corynebacterium sp., Dolosigranulum sp., Streptococcus sp., Lactobacillus sp., or any combination thereof, a total of at least 200 to at least 300 bacterial CFUs of Corynebacterium sp., Dolosigranulum sp., Streptococcus sp., Lactobacillus sp., or any combination thereof, a total of at least 300 to at least 400 bacterial CFUs of Corynebacterium sp., Dolosigranulum sp., Streptococcus sp., Lactobacillus sp., or any combination thereof, a total of at least 400 to at least 500 bacterial CFUs of Corynebacterium sp., Dolosigranulum sp., Streptococcus sp., Lactobacillus sp., or any combination thereof, a total of at least 500 to at least 600 bacterial CFUs of Corynebacterium sp., Dolosigranulum sp., Streptococcus sp., Lactobacillus sp., or any combination thereof, a total of at least 600 to at least 700 bacterial CFUs of Corynebacterium sp., Dolosigranulum sp., Streptococcus sp., Lactobacillus sp., or any combination thereof, a total of at least 700 to at least 800 bacterial CFUs of Corynebacterium sp., Dolosigranulum sp., Streptococcus sp., Lactobacillus sp., or any combination thereof, a total of at least 800 to at least 900 bacterial CFUs of Corynebacterium sp., Dolosigranulum sp., Streptococcus sp., Lactobacillus sp., or any combination thereof, a total of at least 900 to at least 1000 bacterial CFUs of Corynebacterium sp., Dolosigranulum sp., Streptococcus sp., Lactobacillus sp., or any combination thereof, or a total of at least 1000 bacterial CFUs of Corynebacterium sp., Dolosigranulum sp., Streptococcus sp., Lactobacillus sp., or any combination thereof.

In an aspect of a disclosed method of modulating microbial diversity and/or composition, a disclosed therapeutically effective dose can comprise a total of at least 1000 to at least 2000 bacterial CFUs of Corynebacterium sp., Dolosigranulum sp., Streptococcus sp., Lactobacillus sp., or any combination thereof, a total of at least 2000 to at least 3000 bacterial CFUs of Corynebacterium sp., Dolosigranulum sp., Streptococcus sp., Lactobacillus sp., or any combination thereof, a total of at least 3000 to at least 4000 bacterial CFUs of Corynebacterium sp., Dolosigranulum sp., Streptococcus sp., Lactobacillus sp., or any combination thereof, a total of at least 4000 to at least 5000 bacterial CFUs of the Corynebacterium sp., Dolosigranulum sp., Streptococcus sp., Lactobacillus sp., or any combination thereof, a total of at least 5000 to at least 6000 bacterial CFUs of Corynebacterium sp., Dolosigranulum sp., Streptococcus sp., Lactobacillus sp., or any combination thereof, a total of at least 6000 to at least 7000 bacterial CFUs of Corynebacterium sp., Dolosigranulum sp., Streptococcus sp., Lactobacillus sp., or any combination thereof, a total of at least 8000 to at least 9000 bacterial CFUs of Corynebacterium sp., Dolosigranulum sp., Streptococcus sp., Lactobacillus sp., or any combination thereof, or a total of at least 10,000 bacterial CFUs of Corynebacterium sp., Dolosigranulum sp., Streptococcus sp., Lactobacillus sp., or any combination thereof.

In an aspect, a disclosed therapeutically effective dose can comprise a total of at least 10⁴ to at least 10⁵ bacterial CFUs of the one or more probiotics, a total of at least 10⁵ to at least 10⁶ bacterial CFUs of the one or more probiotics, a total of at least 10⁶ to at least 10⁷ bacterial CFUs of the one or more probiotics, a total of at least 10⁷ to at least 10⁸ bacterial CFUs of the one or more probiotics, a total of at least 10⁸ to at least 10⁹ bacterial CFUs of the one or more probiotics, a total of at least 10⁹ to at least 10¹⁰ bacterial CFUs of the one or more probiotics, a total of at least 10¹⁰ to at least 10¹¹ bacterial CFUs of the one or more probiotics, a total of at least 10¹¹ to at least 10¹² bacterial CFUs of the one or more probiotics, a total of at least 10¹² bacterial CFUs of the one or more probiotics, or more than 10¹² bacterial CFUs of one or more disclosed probiotics (such as, for example, at least one strain from the bacterial genus Corynebacterium, at least one strain from the bacterial genus Dolosigranulum, at least one strain from the bacterial genus Streptococcus, and at least one strain from the bacterial genus Lactobacillus, or any combination thereof).

In an aspect, a disclosed therapeutically effective dose can comprise at least 10³, 10⁴, 10⁵, 10⁶, 10⁷, 10⁸, 10⁹, 10¹⁰, 10¹¹, or 10¹² CFUs of a disclosed probiotic or a consortium of disclosed probiotics, preferably at least 1.2×10³, 1.4×10³, 2×10³, or 3×10³ CFUs of a disclosed probiotic or a consortium of disclosed probiotics, at least 5×10³, 5.02×10³, 5.04×10³, 5.2×10³, or 5.4×10³ CFUs of a disclosed probiotic or a consortium of disclosed probiotics, at least 1.02×10⁴, 1.04×10⁴, 1.2×10⁴, 1.4×10⁴, 1.5×10⁴, 2×10⁴, or 3×10⁴ CFUs of a disclosed probiotic or a consortium of disclosed probiotics, at least 5×10⁴, 5.02×10⁴, 5.04×10⁴, 5.2×10⁴, or 5.4×10⁴ CFUs of a disclosed probiotic or a consortium of disclosed probiotics, at least 1.02×10⁵, 1.04×10⁵, 1.2×10⁵, 1.4×10⁵, 1.5×10⁵, 2×10⁵, or 3×10⁵ CFUs of a disclosed probiotic or a consortium of disclosed probiotics, at least 1.02×10⁶, 1.04×10⁶, 1.2×10⁶, 1.4×10⁶, 2×10⁶, or 3×10⁶ CFUs of a disclosed probiotic or a consortium of disclosed probiotics, at least 1.02×10⁷, 1.04×10⁷, 1.2×10⁷, 1.4×10⁷, 2×10⁷, or 3×10⁷ CFUs of a disclosed probiotic or a consortium of disclosed probiotics, at least 1.02×10⁸, 1.04×10⁸, 1.2×10⁸, 1.4×10⁸, 2×10⁸, or 3×10⁸ CFUs of a disclosed probiotic or a consortium of disclosed probiotics, at least 1.02×10⁹, 1.04×10⁹, 1.2×10⁹, 1.4×10⁹, 2×10⁹, or 3×10⁹ CFUs of a disclosed probiotic or a consortium of disclosed probiotics, at least 10¹⁰ CFUs of a disclosed probiotic or a consortium of disclosed probiotics, at least 1.02×10¹⁰, 1.04×10¹⁰, 1.2×10¹⁰, 1.4×10¹⁰, 2×10¹⁰, or 3×10¹⁰ CFUs of a disclosed probiotic or a consortium of disclosed probiotics, at least 1.02×10¹¹, 1.04×10¹¹, 1.2×10¹¹, 1.4×10¹¹, 2×10¹¹, or 3×10¹¹ CFUs of a disclosed probiotic or a consortium of disclosed probiotics, or at least 1.02×10¹², 1.04×10¹², 1.2×10¹², 1.4×10¹², 2×10¹², or 3×10¹² CFUs of a disclosed probiotic or a consortium of disclosed probiotics per dose.

In an aspect, a disclosed therapeutically effective dose can comprise any amount or an unlimited amount of bacterial CFUs of the one or more probiotics.

In an aspect of a disclosed method of modulating microbial diversity and/or composition, a disclosed therapeutically effective dose can comprise Corynebacterium sp., Dolosigranulum sp., Streptococcus sp., and Lactobacillus sp. in a ratio of about 1:0.01:0.01:0.01 to about 1:1:1:1. In an aspect, a disclosed therapeutically effective dose can comprise Corynebacterium sp., Dolosigranulum sp., Streptococcus sp., and Lactobacillus sp. in a ratio of about 1:0.01:0.01:0.01 to about 1:1:1:1. In an aspect, a disclosed therapeutically effective dose can comprise Corynebacterium sp., Dolosigranulum sp., Streptococcus sp., and Lactobacillus sp. in any ratio.

In an aspect, a disclosed therapeutically effective dose can comprise a higher dose of Corynebacterium sp., or a higher dose of Dolosigranulum sp., or a higher dose of Streptococcus sp., or a higher dose of Lactobacillus sp. as compared to any other bacteria in the consortium. In an aspect, a disclosed therapeutically effective dose can comprise a dose of Lactobacillus sp. that is about double that of Corynebacterium sp.

In an aspect, a disclosed method of modulating microbial diversity and/or composition can further comprise treating the subject. In an aspect, treating the subject can comprise treating the subject's respiratory infection. In an aspect, treating the subject can comprise treating the subject's non-respiratory infection.

In an aspect, a disclosed method of modulating microbial diversity and/or composition can comprise further comprising administering to the subject a therapeutically effective amount of one or more anti-bacterial agents. Anti-bacterial agents and combinations of anti-bacterial agents are known to the art and discussed supra in Part VII(C)(1).

In an aspect of a disclosed method of modulating microbial diversity and/or composition, a therapeutically effective amount of a disclosed anti-bacterial agent can comprise a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method of modulating microbial diversity and/or composition can comprise further administering to the subject a therapeutically effective amount of (i) one or more active agents, (ii) one or more biologically active agents, (iii) one or more pharmaceutically active agents, (iv) one or more immune-based therapeutic agents, (v) one or more clinically approved agents, or (vi) any combination thereof. In an aspect, a therapeutically effective amount of an active agent, a biologically active agent, a pharmaceutically active agent, an immune-based therapeutic active agent, a clinically approved agent, or an anti-bacterial agent can be about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, administering a disclosed anti-bacterial agent can comprise systemic or direct administration. In an aspect, administering a disclosed anti-bacterial agent can comprise oral administration, sublingual administration, intravenous administration, intranasal administration, intraperitoneal administration, or any combination thereof. In an aspect, administering a disclosed anti-bacterial agent can be administered by any method of administration disclosed herein. In an aspect, a disclosed anti-bacterial agent can be administered via multiple routes either concurrently or sequentially. A skilled clinician can determine the best route of administration for a disclosed anti-bacterial agent to a subject at a given time. In an aspect, a disclosed anti-bacterial agent can inherently encompass a pharmaceutically acceptable salt thereof.

In an aspect, disclosed method of modulating microbial diversity and/or composition can further comprise increasing the relative abundance of one or more probiotics in the subject's microbiome. In an aspect, disclosed method of modulating microbial diversity and/or composition can further comprise increasing the relative abundance of one or more of Corynebacterium sp., Dolosigranulum sp., Streptococcus sp., and Lactobacillus sp. in the subject's microbiome. In an aspect, disclosed method of modulating microbial diversity and/or composition can further comprise increasing the relative abundance of one or more of at least one strain from the bacterial genus Corynebacterium, at least one strain from the bacterial genus Dolosigranulum, at least one strain from the bacterial genus Streptococcus, and at least one strain from the bacterial genus Lactobacillus, or any combination thereof in the subject's microbiome. In an aspect, disclosed method of modulating microbial diversity and/or composition can further comprise increasing the relative abundance of at least one strain from the bacterial genus Corynebacterium in the subject's microbiome. In an aspect, disclosed method of modulating microbial diversity and/or composition can further comprise increasing the relative abundance of at least one strain from the bacterial genus Dolosigranulum in the subject's microbiome. In an aspect, disclosed method of modulating microbial diversity and/or composition can further comprise increasing the relative abundance of at least one strain from the bacterial genus Streptococcus in the subject's microbiome. In an aspect, disclosed method of modulating microbial diversity and/or composition can further comprise increasing the relative abundance of at least one strain from the bacterial genus Lactobacillus in the subject's microbiome.

In an aspect, a disclosed method of modulating microbial diversity and/or composition can further comprise decreasing the relative abundance of one or more pathogenic bacteria in the subject's microbiome. In an aspect, a disclosed method of modulating microbial diversity and/or composition can further comprise decreasing the relative abundance of S. pneumoniae in the subject's microbiome. In an aspect, a disclosed method of modulating microbial diversity and/or composition can further comprise decreasing the relative abundance of a S. aureus in the subject's microbiome. In an aspect, a disclosed method of modulating microbial diversity and/or composition can further comprise decreasing the relative abundance of Haemophilus influenzae and/or Moraxella catarrhalis in the subject's microbiome.

In an aspect, a disclosed method of modulating microbial diversity and/or composition can further comprise administering to the subject one or more therapeutic agents. In an aspect, a disclosed therapeutic agent can comprise a biologically active agent, a pharmaceutically active agent, an anti-bacterial agent, an anti-fungal agent, an anti-viral agent, a corticosteroid, an analgesic, an immunostimulant, an immune-based product, or any combination thereof. In an aspect of a disclosed method of promoting respiratory health, administering can comprise intranasal administration, oral administration, sublingual administration, or any combination thereof.

In an aspect of a disclosed method of modulating microbial diversity and/or composition, a subject can be a healthy subject, for example, a subject that does not have an infection and/or is not suspected of having an infection.

In an aspect, a subject can be an adult, a child, or an infant. In an aspect, a subject can be a neonate. In an aspect, a subject can be a premature infant. In an aspect, a subject can be immune-compromised. In an aspect, a subject can have diabetes or a chronic disease (e.g., heart disease, kidney disease, or liver disease). In an aspect, a subject can have HIV. In an aspect, a subject can have cancer or has had cancer. In an aspect, a subject can be the recipient of one or more solid organ transplants. In an aspect, a subject can have nephrotic syndrome. In an aspect, a subject can be alcoholic or can smoke cigarettes. In an aspect, a subject can have a chronic lung disease. In an aspect, a disclosed chronic lung disease can comprise emphysema, chronic obstructive lung disease, chronic obstructive pulmonary disease, asthma, or any combination thereof.

In an aspect, a subject can be diagnosed with an infection of one or more parts of the respiratory system. In an aspect, a subject can be suspected of having an infection in one or more parts of the respiratory system. In an aspect, a disclosed therapeutically effective amount of a biotherapeutic can comprise an amount sufficient to inhibit and/or prevent the growth of one or more pathogenic bacteria in one or more parts of the subject's respiratory system. In an aspect, a disclosed therapeutically effective amount of a biotherapeutic can comprise an amount sufficient to inhibit and/or prevent the spread of one or more pathogenic bacteria from a first part of subject's respiratory system to a second part of the subject's respiratory system.

In an aspect, one or more disclosed probiotics can inhibit and/or prevent the growth and/or spread of one or more pathogenic bacteria using competitive adherence and/or non-adherence mechanisms (e.g., secretion of antimicrobial factors). In an aspect, one or more disclosed probiotics can inhibit and/or prevent the adherence and/or colonization of one or more pathogenic bacteria. In an aspect, one or more disclosed probiotics can modulate the endocytosis or paracellular migration of one or more pathogenic bacteria. In an aspect, one or more disclosed probiotics can inhibit and/or prevent the spread of one or more pathogenic bacteria by competitive adherence and/or niche occupancy.

In an aspect, one or more disclosed probiotics can inhibit and/or prevent the growth and/or spread of S. pneumoniae using competitive adherence and/or non-adherence mechanisms (e.g., secretion of antimicrobial factors). In an aspect, one or more disclosed probiotics can inhibit and/or prevent the adherence and/or colonization of S. pneumoniae. In an aspect, one or more disclosed probiotics can modulate the endocytosis or paracellular migration of S. pneumoniae. In an aspect, one or more disclosed probiotics can inhibit and/or prevent the spread S. pneumoniae by competitive adherence and/or niche occupancy.

In an aspect of a disclosed method of modulating microbial diversity and/or composition, one or more disclosed probiotics can secrete one or more factors. In an aspect, a disclosed secreted factor can be species-dependent and/or strain-dependent. In an aspect, a disclosed secreted factor can inhibit and/or prevent the growth and/or spread of one or more pathogenic bacteria. In an aspect, a disclosed secreted factor can inhibit and/or prevent the adherence and/or colonization of one or more pathogenic bacteria. In an aspect, a disclosed secreted factor can modulate the endocytosis or paracellular migration of one or more pathogenic bacteria. In an aspect, a disclosed secreted factor can inhibit and/or prevent the spread of one or more pathogenic bacteria by competitive adherence and/or niche occupancy.

In an aspect of a disclosed method of modulating microbial diversity and/or composition, a disclosed secreted factor can inhibit and/or prevent the growth and/or spread of S. pneumoniae.

In an aspect, a disclosed secreted factor can inhibit and/or prevent the adherence and/or colonization of S. pneumoniae. In an aspect, a disclosed secreted factor can modulate the endocytosis or paracellular migration of S. pneumoniae. In an aspect, a disclosed secreted factor can inhibit and/or prevent the spread of S. pneumoniae by competitive adherence and/or niche occupancy.

In an aspect of a disclosed method of modulating microbial diversity and/or composition, Corynebacterium sp., Dolosigranulum sp., Streptococcus sp., Lactobacillus sp., or any combination thereof can secrete one or more factors that inhibit and/or prevent the growth and/or spread of one or more pathogenic bacteria. In an aspect of a disclosed method of modulating microbial diversity and/or composition, Corynebacterium sp., Dolosigranulum sp., Streptococcus sp., Lactobacillus sp., or any combination thereof can secrete one or more factors that inhibit and/or prevent the adherence and/or colonization of one or more pathogenic bacteria. In an aspect of a disclosed method of modulating microbial diversity and/or composition, Corynebacterium sp., Dolosigranulum sp., Streptococcus sp., Lactobacillus sp., or any combination thereof can secrete one or more secreted factors that modulate the endocytosis or paracellular migration of one or more pathogenic bacteria. In an aspect of a disclosed method of modulating microbial diversity and/or composition, Corynebacterium sp., Dolosigranulum sp., Streptococcus sp., Lactobacillus sp., or any combination thereof can secrete one or more secreted factors inhibit and/or prevent the spread of one or more pathogenic bacteria by competitive adherence and/or niche occupancy. In an aspect of a disclosed method of modulating microbial diversity and/or composition, Corynebacterium sp., Dolosigranulum sp., Streptococcus sp., Lactobacillus sp., or any combination thereof can secrete one or more secreted factors inhibit and/or prevent the growth and/or spread of S. pneumoniae. In an aspect of a disclosed method of modulating microbial diversity and/or composition, Corynebacterium sp., Dolosigranulum sp., Streptococcus sp., Lactobacillus sp., or any combination thereof can secrete one or more factors inhibit and/or prevent the adherence and/or colonization of S. pneumoniae. In an aspect, a disclosed method of modulating microbial diversity and/or composition, Corynebacterium sp., Dolosigranulum sp., Streptococcus sp., Lactobacillus sp., or any combination thereof secrete one or more factors can modulate the endocytosis or paracellular migration of S. pneumoniae. In an aspect of a disclosed method of modulating microbial diversity and/or composition, Corynebacterium sp., Dolosigranulum sp., Streptococcus sp., Lactobacillus sp., or any combination thereof can secrete one or more factors inhibit and/or prevent the spread of S. pneumoniae by competitive adherence and/or niche occupancy.

In an aspect of a disclosed method of modulating microbial diversity and/or composition, Corynebacterium sp. can secrete one or more factors that inhibit and/or prevent the growth and/or spread of one or more pathogenic bacteria. In an aspect of a disclosed method of modulating microbial diversity and/or composition, Corynebacterium sp. can secrete one or more factors that inhibit and/or prevent the adherence and/or colonization of one or more pathogenic bacteria. In an aspect of a disclosed method of modulating microbial diversity and/or composition, Corynebacterium sp. can secrete one or more secreted factors that modulate the endocytosis or paracellular migration of one or more pathogenic bacteria. In an aspect of a disclosed method of modulating microbial diversity and/or composition, Corynebacterium sp. can secrete one or more secreted factors that inhibit and/or prevent the spread of one or more pathogenic bacteria by competitive adherence and/or niche occupancy.

In an aspect of a disclosed method of modulating microbial diversity and/or composition, Corynebacterium sp. can secrete one or more secreted factors that inhibit and/or prevent the growth and/or spread of S. pneumoniae. In an aspect of a disclosed method of modulating microbial diversity and/or composition, Corynebacterium sp. can secrete one or more factors that inhibit and/or prevent the adherence and/or colonization of S. pneumoniae. In an aspect of a disclosed method of modulating microbial diversity and/or composition, Corynebacterium sp. can secrete one or more factors that modulate the endocytosis or paracellular migration of S. pneumoniae. In an aspect of a disclosed method of modulating microbial diversity and/or composition, Corynebacterium sp. can secrete one or more factors that inhibit and/or prevent the spread of S. pneumoniae by competitive adherence and/or niche occupancy.

In an aspect, a disclosed method of modulating microbial diversity and/or composition can comprise a 10%, 20%, 30%, 40%, 50%, 60%, 70%, 80%, 90%, 100%, or any amount of inhibiting and/or preventing the growth and/or spread of one or more pathogenic bacteria (e.g., S. pneumoniae) when compared to a control subject (such as, for example, a subject that has not received a disclosed biotherapeutic). In an aspect, a disclosed method of modulating microbial diversity and/or composition can comprise a 10-20%, 20-30%, 30-40%, 40-50%, 50-60%, 60-70%, 70-80%, 80-90%, or 90-100% or any amount of inhibiting and/or preventing the growth and/or spread of one or more pathogenic bacteria (e.g., S. pneumoniae) when compared to a control subject (such as, for example, a subject that has not received a disclosed biotherapeutic).

In an aspect, a disclosed method of modulating microbial diversity and/or composition can comprise a 10%, 20%, 30%, 40%, 50%, 60%, 70%, 80%, 90%, 100%, or any amount of inhibiting and/or preventing the adherence and/or colonization of one or more pathogenic bacteria (e.g., S. pneumoniae) when compared to a control subject (such as, for example, a subject that has not received a disclosed biotherapeutic). In an aspect, a disclosed method of modulating microbial diversity and/or composition can comprise a 10-20%, 20-30%, 30-40%, 40-50%, 50-60%, 60-70%, 70-80%, 80-90%, or 90-100% or any amount of inhibiting and/or preventing the adherence and/or colonization of one or more pathogenic bacteria (e.g., S. pneumoniae) when compared to a control subject (such as, for example, a subject that has not received a disclosed biotherapeutic).

In an aspect, a disclosed method of modulating microbial diversity and/or composition can comprise a 10%, 20%, 30%, 40%, 50%, 60%, 70%, 80%, 90%, 100%, or any amount of modulating the endocytosis or paracellular migration of one or more pathogenic bacteria (e.g., S. pneumoniae) when compared to a control subject (such as, for example, a subject that has not received a disclosed biotherapeutic). In an aspect, a disclosed method of modulating microbial diversity and/or composition can comprise a 10-20%, 20-30%, 30-40%, 40-50%, 50-60%, 60-70%, 70-80%, 80-90%, or 90-100% or any amount of modulating the endocytosis or paracellular migration of one or more pathogenic bacteria (e.g., S. pneumoniae) when compared to a control subject (such as, for example, a subject that has not received a disclosed biotherapeutic).

In an aspect, a disclosed method of modulating microbial diversity and/or composition can comprise a 10%, 20%, 30%, 40%, 50%, 60%, 70%, 80%, 90%, 100%, or any amount of inhibiting and/or preventing the spread of one or more pathogenic bacteria (e.g., S. pneumoniae) by competitive adherence and/or niche occupancy when compared to a control subject (such as, for example, a subject that has not received a disclosed biotherapeutic). In an aspect, a disclosed method of modulating microbial diversity and/or composition can comprise a 10-20%, 20-30%, 30-40%, 40-50%, 50-60%, 60-70%, 70-80%, 80-90%, or 90-100% or any amount of inhibiting and/or preventing the spread of one or more pathogenic bacteria (e.g., S. pneumoniae) by competitive adherence and/or niche occupancy when compared to a control subject (such as, for example, a subject that has not received a disclosed biotherapeutic).

In an aspect, a disclosed method of modulating microbial diversity and/or composition can comprise repeating the administering of one or more disclosed biotherapeutics, one or more disclosed secreted factors, a disclosed consortium of probiotics, one or more disclosed probiotics, or any combination thereof. For example, in an aspect, repeating the administering can comprise administering one or more times daily (e.g., 1, 2, 3, or 4 times). In an aspect, repeating the administering can comprise administering on several consecutive days (e.g., for 1, 2, 3, 4, 5, 6, 7, 8, 9, or 10 days, or more than 10 days), weeks (e.g., for 1, 2, 3, 4, 5, 6, 7, 8, 9, or 10 weeks, or more than 10 weeks), or months (e.g., for 1, 2, 3, 4, 5, or 6 months, or more than 6 months). In an aspect, a disclosed biotherapeutic, a disclosed secreted factor, a disclosed consortium of probiotics, a disclosed probiotic, or any combination thereof can be administered 1-3 times per day for about 7 to 21 days.

In an aspect, a disclosed method of modulating microbial diversity and/or composition can comprise monitoring the subject. In an aspect, monitoring the subject can comprise monitoring the subject for the development of adverse effects. In an aspect, in the absence of adverse effects, a disclosed method of modulating microbial diversity and/or composition can comprise continuing to treat the subject. In an aspect, continuing to treat the subject can comprise continuing to administer to the subject one or more disclosed biotherapeutics, one or more disclosed secreted factors, a disclosed consortium of probiotics, one or more disclosed probiotics, or any combination thereof. In an aspect, in the presence of adverse effects, a disclosed method of modulating microbial diversity and/or composition can comprise modifying one or more steps of the method.

In an aspect, modifying one or more steps of a disclosed method can comprise modifying the administering step. In an aspect, modifying the administering step can comprise changing the amount of one or more disclosed biotherapeutics, one or more disclosed secreted factors, a disclosed consortium of probiotics, one or more disclosed probiotics, or any combination thereof administered to the subject, changing the frequency of administration of one or more disclosed biotherapeutics, one or more disclosed secreted factors, a disclosed consortium of probiotics, one or more disclosed probiotics, or any combination thereof, changing the duration of administration of one or more disclosed biotherapeutics, one or more disclosed secreted factors, a disclosed consortium of probiotics, one or more disclosed probiotics, or any combination thereof, changing the route of administration of one or more disclosed biotherapeutics, one or more disclosed secreted factors, a disclosed consortium of probiotics, one or more disclosed probiotics, or any combination thereof, or any combination of disclosed changes.

In an aspect, a disclosed method of modulating microbial diversity and/or composition can comprise manipulating the microbiome to inhibit and/or prevent infection caused by one or more pathogenic bacteria, such as an infection caused by S. pneumoniae. In an aspect, a disclosed method of modulating microbial diversity and/or composition can comprise manipulating the microbiome to decrease and/or minimize the risk of infection caused by one or more pathogenic bacteria, such as an infection caused by S. pneumoniae. For example, in an aspect, when the relative abundance of one or more disclosed Corynebacterium sp. is below a threshold level of 1.2×10⁶ 16S rRNA gene copies per biological sample, then the risk of colonization by S. pneumoniae is increased and/or higher and/or enhanced. In an aspect, when the relative abundance of Corynebacterium sp. is below a threshold level of 1.2×10⁶ 16S rRNA gene copies per biological sample, then a disclosed method can further comprise administering and/or continuing to administer a therapeutically effective amount of a disclosed biotherapeutic. In an aspect, when the relative abundance of Corynebacterium sp. is above 1.2×10⁶ 16S rRNA gene copies per biological sample, then a disclosed method can further comprise continuing to monitor the subject.

In an aspect, a disclosed method of promoting respiratory health can comprise manipulating the microbiome to inhibit and/or prevent infection caused by one or more pathogenic bacteria, such as an infection caused by S. pneumoniae. In an aspect, a disclosed method of promoting respiratory health can comprise manipulating the microbiome to decrease and/or minimize the risk of infection caused by one or more pathogenic bacteria, such as an infection caused by S. pneumoniae. For example, in an aspect, when the relative abundance of one or more disclosed Corynebacterium sp. is below a threshold level of 1.2×10⁶ 16S rRNA gene copies per biological sample, then the risk of colonization by S. pneumoniae is increased and/or higher and/or enhanced. In an aspect, when the relative abundance of Corynebacterium sp. is below a threshold level of 1.2×10⁶ 16S rRNA gene copies per biological sample, then a disclosed method can further comprise administering and/or continuing to administer a therapeutically effective amount of a disclosed biotherapeutic. In an aspect, when the relative abundance of Corynebacterium sp. is above 1.2×10⁶ 16S rRNA gene copies per biological sample, then a disclosed method can further comprise continuing to monitor the subject.

For example, in an aspect, when the relative abundance of one or more disclosed Corynebacterium sp. is less than about 50%, about 60%, about 70%, about 80%, or 90% of the microbiome, then the risk of colonization by S. pneumoniae is increased and/or higher and/or enhanced. For example, in an aspect, when the relative abundance of one or more disclosed Corynebacterium sp. is about 50% to about 60%, about 60% to about 70%, about 70% to about 80%, about 80% to about 90% of the microbiome, then the risk of colonization by S. pneumoniae is increased and/or higher and/or enhanced. In an aspect, when the relative abundance of Corynebacterium sp. is about 50% to about 60%, about 60% to about 70%, about 70% to about 80%, about 80% to about 90% of the microbiome, then a disclosed method can further comprise administering and/or continuing to administer a therapeutically effective amount of a disclosed biotherapeutic. In an aspect, when the relative abundance of Corynebacterium sp. is less than about 50%, about 60%, about 70%, about 80%, or 90% of the microbiome, then a disclosed method can further comprise administering and/or continuing to administer a therapeutically effective amount of a disclosed biotherapeutic. In an aspect, when the relative abundance of Corynebacterium sp. is about 50% to about 60%, about 60% to about 70%, about 70% to about 80%, about 80% to about 90% of the microbiome, then a disclosed method can further comprise continuing to monitor the subject. In an aspect, when the relative abundance of Corynebacterium sp. is less than about 50%, about 60%, about 70%, about 80%, or 90%, then a disclosed method can further comprise continuing to monitor the subject.

In an aspect, a disclosed method of modulating microbial diversity and/or composition can comprise subjecting the subject to one or more invasive or non-invasive diagnostic assessments.

Diagnostic assessments are known to the art. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination thereof. In an aspect, a disclosed invasive diagnostic assessment can comprise a tissue biopsy or exploratory surgery.

In an aspect of a disclosed method of modulating microbial diversity and/or composition, techniques to monitor, measure, and/or assess the restoring one or more aspects of respiratory health can comprise qualitative (or subjective) means as well as quantitative (or objective) means. These means are known to the skilled person. For example, representative regulated variables and sensors relating to systemic homeostasis are discussed supra.

In an aspect of a disclosed method of modulating microbial diversity and/or composition, a disclosed biotherapeutic, a disclosed secreted factor, a disclosed consortium of probiotics, a disclosed probiotic, or any combination thereof can be administered in the absence of identifying the one or more pathogenic bacteria and/or in the absence of characterizing the microbiome. In an aspect of a disclosed method, a disclosed biotherapeutic, a disclosed secreted factor, a disclosed consortium of probiotics, a disclosed probiotic, or any combination thereof can be administered on demand.

In an aspect of a disclosed method of modulating microbial diversity and/or composition, a disclosed biotherapeutic can be formulated for administration as a pharmaceutical formulation.

In an aspect, a disclosed pharmaceutical formulation comprising a probiotic, a consortium of probiotics, factors secreted from a probiotic, factors secreted from a consortium of probiotics, or any combination thereof, and a pharmaceutically acceptable carrier and/or excipient can be used in any disclosed method of modulating microbial diversity and/or composition. Any pharmaceutical formulation disclosed herein can be used in a disclosed method of modulating microbial diversity and/or composition.

### H. Kits

Disclosed herein is a kit comprising one or more of a disclosed biotherapeutic, a disclosed secreted factor, a disclosed consortium of probiotics, a disclosed probiotic, or any combination.

In an aspect, a disclosed kit can comprise a disclosed pharmaceutical formulation comprising a disclosed biotherapeutic, a disclosed secreted factor, a disclosed consortium of probiotics, a disclosed probiotic, or any combination thereof.

Disclosed herein is a kit comprising at least one strain from the bacterial genus Corynebacterium. Disclosed herein is a kit comprising at least one strain from the bacterial genus Dolosigranulum. Disclosed herein is a kit comprising at least one strain from the bacterial genus Streptococcus. Disclosed herein is a kit comprising at least one at least one strain from the bacterial genus Lactobacillus. Disclosed herein is a kit comprising at least one strain from the bacterial genus Corynebacterium, at least one strain from the bacterial genus Dolosigranulum, at least one strain from the bacterial genus Streptococcus, and at least one strain from the bacterial genus Lactobacillus.

Disclosed herein is a kit comprising a pharmaceutical formulation comprising at least one Corynebacterium sp. Disclosed herein is a kit comprising a pharmaceutical formulation comprising at least one Dolosigranulum sp. Disclosed herein is a kit comprising a pharmaceutical formulation comprising at least one Streptococcus sp. Disclosed herein is a kit comprising a pharmaceutical formulation comprising at least one Lactobacillus sp. Disclosed herein is a kit comprising a pharmaceutical formulation comprising at least one Corynebacterium sp., at least one Dolosigranulum sp., at least one Streptococcus sp., and at least one Lactobacillus sp.

In an aspect, a disclosed kit can further comprise one or more anti-bacterial agents, one or more therapeutic agents, or any combination thereof. “Anti-bacterial agents” and “Therapeutic Agents” are known to the art and are described supra. For example, anti-bacterial agents and combinations of anti-bacterial agents are known to the art and discussed supra in Part VII(C)(1).

In an aspect, the one or more disclosed biotherapeutics, disclosed secreted factors, disclosed consortiums of probiotics, disclosed probiotics, or any combination thereof can promote respiratory health in a subject, can reduce the risk of developing an infection caused by pathogenic bacteria, can reduce the risk of developing a S. pneumoniae infection in a subject, can treat and/or prevent S. pneumoniae colonization and/or infection, can modulate microbial diversity and/or composition, or any combination thereof. In an aspect, the one or more disclosed therapeutic agents in a disclosed kit can treat, prevent, inhibit, and/or ameliorate one or more comorbidities in a subject.

In an aspect, a disclosed kit can comprise at least two components constituting the kit.

Together, the components constitute a functional unit for a given purpose (such as, for example, treating a subject diagnosed with or suspected of having an infection such as a respiratory infection). Individual member components may be physically packaged together or separately.

For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise (i) one or more of a disclosed biotherapeutic, a disclosed secreted factor, a disclosed consortium of probiotics, a disclosed probiotic, a disclosed pharmaceutical formulation, or any combination thereof, and (ii) a label or package insert with instructions for use. In an aspect, suitable containers include, for example, bottles, vials, syringes, blister pack, etc. The containers can be formed from a variety of materials such as glass or plastic. The container can hold one or more of a disclosed biotherapeutic, a disclosed secreted factor, a disclosed consortium of probiotics, a disclosed probiotic, or any combination, a disclosed pharmaceutical formulation, or any combination thereof, and can have a sterile access port (for example the container may be an intravenous solution bag or a vial having a stopper pierceable by a hypodermic injection needle). The label or package insert can indicate one or more of a disclosed biotherapeutic, a disclosed secreted factor, a disclosed consortium of probiotics, a disclosed probiotic, or any combination, a disclosed pharmaceutical formulation, or any combination thereof can be used for promoting respiratory health in a subject, for reducing the risk of developing an infection caused by pathogenic bacteria, for reducing the risk of developing a S. pneumoniae infection in a subject, for treating and/or preventing S. pneumoniae colonization and/or infection, for modulating microbial diversity and/or composition, or any combination thereof. A kit can comprise additional components necessary for administration such as, for example, other buffers, diluents, filters, needles, and syringes. In an aspect, a disclosed kit can be used to promote respiratory health in a subject, to reduce the risk of developing an infection caused by pathogenic bacteria, to reduce the risk of developing a S. pneumoniae infection in a subject, to treat and/or prevent S. pneumoniae colonization and/or infection, to modulate microbial diversity and/or composition, or any combination thereof

### I. Miscellaneous

Disclosed herein is a method of preventing and/or reducing the risk of a respiratory infection in a subject, the method comprising: (a) obtaining a biological sample from the subject; (b) generating a microbiome dataset based upon sequencing the microorganism portion of the sample; (c) determining the level of Corynebacterium in the sample as compared to a control sample; and (d) increasing the relative abundance of Corynebacterium in the subject if the level of Corynebacterium is less than that of the control.

In an aspect of a disclosed method of preventing and/or reducing the risk of a respiratory infection, the respiratory infection can comprise bacterial pneumonia. In an aspect, a disclosed bacterial pneumonia can be caused by Staphylococcus aureus including but not limited to methicillin-resistant Staphylococcus aureus (MRSA), Streptococcus pneumoniae (S. pneumoniae) including penicillin-resistant Streptococcus pneumoniae (PRSP), Haemophilus influenzae, Moraxella catarrhalis, Klebsiella pneumoniae, Legionella pneumophila, Chlamydophila pneumoniae, Mycoplasma pneumoniae, Chlamydophila psittaci, Coxiella burnetii, Escherichia coli, or any combination thereof. In an aspect of a disclosed method of preventing and/or reducing the risk of a respiratory infection, the bacterial pneumonia can be caused by S. pneumoniae. In an aspect of a disclosed method of preventing and/or reducing the risk of a respiratory infection, a disclosed biological sample can comprise a nasal swab or nasal lavage. In an aspect of a disclosed method of preventing and/or reducing the risk of a respiratory infection, a disclosed microbiome can comprise the nasopharyngeal microbiome.

Disclosed herein is a method of determining the risk of a subject developing a respiratory infection, the method comprising: (a) obtaining a biological sample from the subject; (b) generating a microbiome dataset based upon sequencing the microorganism portion of the sample; (c) determining the level of Corynebacterium in the sample as compared to a control sample wherein a relative abundance of Corynebacterium of less than that of the control is indicative of a higher likelihood of developing a respiratory infection; and (d) increasing the relative abundance of Corynebacterium in the subject if the level of Corynebacterium is less than that of the control.

In an aspect of a disclosed method of determining the risk of a subject developing a respiratory infection, the respiratory infection can comprise bacterial pneumonia. In an aspect, a disclosed bacterial pneumonia can be caused by Staphylococcus aureus including but not limited to methicillin-resistant Staphylococcus aureus (MRSA), Streptococcus pneumoniae (S. pneumoniae) including penicillin-resistant Streptococcus pneumoniae (PRSP), Haemophilus influenzae, Moraxella catarrhalis, Klebsiella pneumoniae, Legionella pneumophila, Chlamydophila pneumoniae, Mycoplasma pneumoniae, Chlamydophila psittaci, Coxiella burnetii, Escherichia coli, or any combination thereof. In an aspect of a disclosed method of determining the risk of a subject developing a respiratory infection, the bacterial pneumonia can be caused by S. pneumoniae. In an aspect of a disclosed method of determining the risk of a subject developing a respiratory infection, a disclosed biological sample can comprise a nasal swab or nasal lavage. In an aspect of a disclosed method of determining the risk of a subject developing a respiratory infection, a disclosed microbiome can comprise the nasopharyngeal microbiome.

Disclosed herein is a method of preventing and/or reducing the risk of a respiratory infection in an infant subject, the method comprising: (a) obtaining a biological sample from the subject; (b) generating a microbiome dataset based upon sequencing the microorganism portion of the sample; (c) determining the level of Corynebacterium in the sample as compared to a control sample; and (d) increasing the relative abundance of Corynebacterium in the subject by administering breast milk to the subject if the level of Corynebacterium is less than that of the control.

In an aspect of a disclosed method of preventing and/or reducing the risk of a respiratory infection in an infant subject, the respiratory infection can comprise bacterial pneumonia. In an aspect, a disclosed bacterial pneumonia can be caused by Staphylococcus aureus including but not limited to methicillin-resistant Staphylococcus aureus (MRSA), Streptococcus pneumoniae (S. pneumoniae) including penicillin-resistant Streptococcus pneumoniae (PRSP), Haemophilus influenzae, Moraxella catarrhalis, Klebsiella pneumoniae, Legionella pneumophila, Chlamydophila pneumoniae, Mycoplasma pneumoniae, Chlamydophila psittaci, Coxiella burnetii, Escherichia coli, or any combination thereof. In an aspect of a disclosed method of preventing and/or reducing the risk of a respiratory infection in an infant subject, the bacterial pneumonia can be caused by S. pneumoniae. In an aspect of a disclosed method of preventing and/or reducing the risk of a respiratory infection in an infant subject, a disclosed biological sample can comprise a nasal swab or nasal lavage. In an aspect of a disclosed method of preventing and/or reducing the risk of a respiratory infection in an infant subject, a disclosed microbiome can comprise the nasopharyngeal microbiome.

### EXAMPLES

There is thus an urgent need to develop alternative approaches to preventing infections caused by S. pneumoniae.

Streptococcus pneumoniae (also known as pneumococcus) is a leading cause of severe infections among children and adults, with the highest incidence of pneumococcal disease occurring during infancy (1). S. pneumoniae causes a broad range of infections ranging from mild respiratory illnesses, such as acute otitis media and acute sinusitis, to invasive pneumococcal disease (IPD), which includes serious illnesses such as bloodstream infection and meningitis (Bogaert D, et al (2004) Lancet Infect Dis. 4(3):144-154). Globally, S. pneumoniae is responsible for more than 300,000 child deaths each year, the overwhelming majority of which result from pneumonia (Wahl B, et al. (2018) Lancet Glob Health. 6(7):e744-e757) and occur in low- and middle-income countries (McAllister D A, et al. (2019) Lancet Glob Health. 7(1):e47-e57). Colonization of the nasopharynx precedes infections caused by S. pneumoniae and occurs in 25%-65% of children and <15% of adults, with substantial variation in prevalence by geography and socioeconomic factors (5-9). Pneumococcal conjugate vaccines effectively prevent IPD caused by vaccine serotypes (Kaplan S L, et al. (2004) Pediatrics. 113(3):443-449; Hammitt L L, et al. (2019) The Lancet. 393(10186):2146-2154) but are less effective in preventing non-invasive infections, such as pneumonia (12, 13) and otitis media (Eskola J, et al. (2001) N Engl J Med. 344(6):403-409). Moreover, the introduction of pneumococcal conjugate vaccines has been followed by the emergence of non-vaccine serotypes, some of which are highly virulent and multidrug resistant (15-17), and nonencapsulated pneumococci (Bradshaw J L, et al. (2019) PLoS Pathog. 15(8):e1007911), which together threaten to compromise the long-term benefits of these vaccines (19, 20).

A complex microbial community resides in the upper respiratory tract and has co-evolved with humans (Zaneveld J, et al. (2008) Curr Opin Chem Biol. 12(1):109-114). Over the last decade, accumulating evidence has emerged for this microbiome's role in the pathogenesis of respiratory infections (de Steenhuijsen Piters W A, et al. (2020) Cell Host Microbe. 28(2):223-232), with microbial communities at distinct anatomical sites within the upper respiratory tract contributing to resistance to colonization or infection by specific pathogens. Within the nasopharynx, resident microbes may resist colonization by S. pneumoniae through competition for nutrients and adhesion sites, secretion of antimicrobial factors, and modulation of host immune responses (Bogaert D, et al. (2004) The Lancet. 363(9424):1871-1872). Most prior studies focused on associations between S. pneumoniae and colonization by other major respiratory pathobionts, demonstrating primarily positive associations with Haemophilus influenzae and Moraxella catarrhalis (24-26) and negative associations with Staphylococcus aureus (25, 27).

More recently, negative associations were reported between S. pneumoniae and other streptococcal species (28, 29), Lactobacillus species (30, 31), non-diphtheriae Corynebacterium species (6, 32, 33), and Dolosigranulum pigrum (32, 33). The interspecies interactions that underlie these associations are often bidirectional, occur by diverse mechanisms, and may vary based on the local microenvironment. For instance, H. influenzae can inhibit S. pneumoniae by downregulating expression of pneumococcal adherence factors (Cope E K, et al. (2011) PloS one. 6(12):e28523) or by stimulating complement-dependent phagocytosis of S. pneumoniae (Lysenko E S, et al. (2005) PLoS pathogens. 1(1):e1). Still, these species often persist together in a multi-species biofilm that can confer protection to S. pneumoniae from antibiotics (36, 37). Similarly, inhibition of pneumococcal growth by D. pigrum appears to require the presence of specific Corynebacterium species (Brugger S D, et al. (2020) Msphere. 5(5)). At the same time, Corynebacterium accolens inhibits S. pneumoniae through hydrolysis of free fatty acids from host skin surface triacylglycerols (Bomar L, et al. (2016) mBio. 7(1):e01725-15). Although these laboratory studies have furthered the understanding of respiratory microbial community ecology, the extent to which these interspecies interactions contribute to population-level trends in S. pneumoniae colonization is unknown.

As described above, colonization of the nasopharynx is a necessary precursor to infections caused by S. pneumoniae, and is particularly prevalent among young children. Notably, the nasopharyngeal microbiome undergoes rapid shifts in composition during early childhood (39-41), primarily driven by environmental factors, such as delivery mode (42, 43), infant feeding practices (41, 44), contact with other children (39), season (40, 45), and antibiotic exposures (39).

Previous studies that evaluated associations between the nasopharyngeal microbiome and S. pneumoniae colonization in children were cross-sectional (6, 32, 33), and few data are available from low- and middle-income countries, where >80% of child deaths from S. pneumoniae occur (3) and household (46, 47) and environmental (48) exposures may differ from those in high-income countries. Longitudinal studies in these settings are necessary to determine the impact of environmental exposures on the developing infant nasopharyngeal microbiome and to identify microbiome features that facilitate or inhibit S. pneumoniae colonization.

This study sought to identify interspecies interactions that modify the risk of pneumococcal colonization during infancy and to describe nasopharyngeal microbiome development during the first year of life in a sub-Saharan African setting. The nasopharyngeal microbiomes of 179 mother-infant dyads in Botswana was studied using 16S ribosomal RNA (rRNA) gene sequencing and S. pneumoniae colonization with a species-specific PCR assay was identified. This work identified changes in microbiome diversity and composition during infancy, evaluated associations between the nasopharyngeal microbiome and pneumococcal colonization risk, and identified environmental factors that influenced nasopharyngeal microbiome composition during infancy.

The Examples that follow are illustrative of specific aspects of the invention, and various uses thereof. They set forth for explanatory purposes only and are not to be taken as limiting the invention.

### Materials and Methods Employed in Specific Examples

### 1. Clinical Settings

Botswana is a landlocked country in southern Africa with a semi-arid climate and a rainy summer season that typically occurs from November to March. The country's under-five child mortality rate was estimated to be 41.6 per 1,000 live births in 2019 (UN Inter-agency Group for Child Mortality Estimation. Levels & trends in child mortality, report 2020). Haemophilus influenzae type B (Hib) and 13-valent pneumococcal conjugate (PCV-13) vaccinations were included in the national immunization program in November 2010 and July 2012, respectively. Complete vaccine series coverage rates in 2019 were estimated to be 95% for Hib and 92% for PCV-13 (United Nations Children's Fund, World Health Organization. Botswana: WHO and UNICEF estimates of immunization coverage, 2019 revision). Botswana's capital and largest city, Gaborone, is in the country's South-East district and has a population of 231,626 based on a census conducted in 2011 (Statistics Botswana. 2011 Population and Housing Census Analytical Report).

The HIV prevalence among adults 15 to 49 years of age in Botswana was 20.7% in 2019 (Joint United Nations Programme on HIV/AIDS. UNAIDS estimates 2019: Botswana). More than 95% of pregnant women with HIV in Botswana receive antiretroviral therapy, and the mother-to-child HIV transmission rate is estimated to be less than 2% (Joint United Nations Programme on HIV/AIDS. UNAIDS estimates 2019: Botswana).

### 2. Data and Biospecimen Collection

Mother-infant dyads (n=178) were enrolled within 72 hours of delivery between February 2016 and December 2018 at three sites: (i) a referral hospital in Gaborone, (ii) a public clinic in a low-income urban neighborhood in Gaborone, and (iii) a public clinic in a rural village located˜15 km outside of Gaborone.

Exclusion criteria included maternal age less than 18 years, infant birth weight less than 2,000 g, multiple gestation pregnancy, and caesarian delivery. Participants were seen for monthly study visits until the infant was six months of age and every other month thereafter until the infant was 12 months of age. At all study visits, a caregiver questionnaire was administered, and nasopharyngeal swabs were collected from mothers and infants by trained study personnel. Nasopharyngeal samples were placed directly into MSwab medium (Copan Italia), transported to the National Health Laboratory in Gaborone, and frozen within 4 hours of collection to −80° C. Testing for S. pneumoniae was performed using a quantitative PCR assay targeting the autolysin gene (lytA), as previously described (6, 84). A dried blood spot was collected from HIV-exposed infants by heel prick at two months of age and, for infants who were breastfed for any duration, again at 12 months of age. These samples were tested for HIV-1 DNA using the Cobas AmpliPrep/Cobas TaqMan HIV-1 Qualitative Assay, version 2.0 (Roche) (Gueye S B, et al. (2016) J Virol Methods. 229:12-15). All study participants or their legal guardians provided written informed consent to participate in this study. The study protocol was approved by the Botswana Ministry of Health, the Princess Marina Hospital ethics committee, and institutional review boards at the University of Pennsylvania, Children's Hospital of Philadelphia, McMaster University, and Duke University.

### 3. Processing of Nasopharyngeal Samples for 16S Ribosomal RNA Gene Sequencing

The Duke Microbiome Core Facility extracted DNA from nasopharyngeal samples using Powersoil Pro extraction kits (Qiagen) following the manufacturer's instructions. DNA concentrations were determined using Qubit dsDNA high-sensitivity assay kits (Thermo Fisher Scientific). Negative extraction and PCR controls were amplified with all four batches of samples included in analyses to evaluate for background contamination. For the first two sample batches, these negative controls were verified to not have visible bands on gel electrophoresis. For the final two sample batches, sequencing was performed on these negative control samples. Bacterial community composition was characterized by PCR amplification of the V4 variable region of the 16S rRNA gene using the forward primer 515 and the reverse primer 806 following the Earth Microbiome Project protocol (Gilbert J A, et al. (2010) Stand Genomic Sci. 3(3):243-248). These primers carry unique barcodes that allow for multiplexed sequencing. Equimolar 16S rRNA PCR products from all samples were quantified and pooled prior to sequencing. Sequencing was performed by the Duke Sequencing and Genomic Technologies Core Facility on a MiSeq instrument (Illumina, Inc.) configured for 250 base-pair paired-end sequencing. Raw sequences were trimmed using Trimmomatic version 0.36 (Bolger A M, et al. (2014) Bioinformatics. 30(15):2114-2120), demultiplexed using QIIMIE2 tools (Caporaso J G, et al. (2010) Nature methods. 7(5):335-336), and analyzed through a pipeline that used DADA2 version 1.16 (Callahan B J, et al. (2016) Nature methods. 13(7):581).

ASVs were given taxonomic assignments based on alignment to the expanded Human Oral Microbiome Database (Escapa I, et al. (2020) Microbiome. 8:1-16). The most abundant genera in negative control samples were Delftia, Brevundimonas, Bacteroides, and Leptothrix. Presumed reagent contaminant ASVs (n=248) were identified and removed based on presence in negative control samples or negative correlation with DNA concentration using the frequency method (threshold=0.10) implemented in the decontam R package version 1.12.0 (Davis N M, et al. (2018) Microbiome. 6(1):226). Samples with less than 1,000 sequencing reads after quality filtering were excluded. Sequencing reads were classified into 7,167 ASVs representing 200 genera from 8 phyla. For each of the 279 ASVs assigned to the genus Corynebacterium, a standard nucleotide REFSEQ BLAST search was performed using the National Center for Biotechnology Information's Bacteria and Archaea 16S ribosomal RNA project database (Camacho C, et al. (2009) BMC Bioinformatics. 10:421). Species information was assigned to ASVs using a best-hit approach based on the E value with a minimum percent identity of 97%.

### 4. Statistical Methods for Analyzing Infant Nasopharyngeal Microbiome Diversity and Stability

Alpha (Shannon and Chaol indices) and beta diversity (Bray-Curtis dissimilarity) was calculated using the phyloseq R package version 1.36.0 (McMurdie P J, et al. (2013) PloS one. 8(4):e61217). Wilcoxon signed-rank tests were used to compare microbiome alpha diversity across infant ages and between paired infant and maternal samples. To evaluate associations between infant age and alpha diversity measures, negative binomial mixed effect models with subject as a random effect were used to account for repeated sampling of individuals. Beta diversity with PERMANOVA was compared using the adonis function within the vegan R package version 2.5.7 (Oksanen J, et al. (2007) Community Ecology Package. 10(631-637):719); for comparisons of infant samples collected at different ages, a variable containing a unique identifier for each infant was included as a blocking variable (Anderson M J, et al. (2001) Austral Ecology. 26(1):32-46).

For initial analyses of specific microbiome features, ASVs were aggregated at the genus level. Samples were classified as dominated when 50% or more of the sequencing reads generated from this sample were assigned to a single genus. Samples dominated by a genus other than the six most highly abundant genera were classified in a single “other” category. Samples for which no single genus accounted for the majority of the sequencing reads were classified as “biodiverse.” A sample's microbiome profile was considered to be “stable” when the next visit's sample from that infant was classified as the same biotype. Logistic regression was then used to evaluate associations between specific nasopharyneal biotypes and microbiome stability, adjusting for infant age in days.

### 5. Sequence Statistical Analyses for Analyzing Infant Nasopharyngeal Microbiome Composition

MaAsLin2 version 1.6.0 (Mallick H, et al. (2021) bioRxiv. 2021.03.28.437378) was used to fit log-transformed linear mixed models evaluating associations between sociodemographic factors and environmental exposures and the relative abundances of bacterial genera within the infant nasopharyngeal microbiome. These analyses considered the following variables identified based on a literature review: sex, low birth weight (<2500 g), HIV exposure status, location of residence (urban vs. rural), household use of solid fuels, number of other child household members (<5 years of age), season (summer vs. winter), breastfeeding, number of PCV-13 doses, and systemic antibiotic exposures (39-41, 44, 45, 95-97). MaAsLin2 analyses were limited to bacterial genera present in at least 10% of nasopharyngeal samples. The comparisons were corrected for the false discovery rate using the Benjamini-Hochberg procedure and a q value threshold for significance of 0.20. The models included subject as a random effect. To identify compositional features of the nasopharyngeal microbiome that influence the risk of pneumococcal colonization, a Cox proportional hazards model was fit evaluating the association between nasopharyngeal microbiome biotype and S. pneumoniae colonization detected at the subsequent study visit.

Given the observed negative association between a Corynebacterium-dominant biotype and pneumococcal colonization in this model, additional Cox proportional hazards models were fit to evaluate the association between the relative abundance of Corynebacterium, modeled on the genus level and separately for specific highly abundant species, and S. pneumoniae colonization. Two (1%) infants who were colonized with S. pneumoniae at the birth visit were excluded from these analyses. In addition, infants who were not colonized with S. pneumoniae at any study visit were censored at the last visit for which S. pneumoniae colonization data were available. Time was modeled as a continuous variable corresponding to infant age in days. All models were implemented using the survival R package version 3.2.11 (Therneau T (2015). A Package for Survival Analysis in S. version 2.38) and were adjusted for all previously specified sociodemographic factors and environmental exposures, with the season, breastfeeding, PCV-13 doses, and systemic antibiotic exposures modeled as time-dependent covariates.

### 6. Laboratory Experiments Evaluating for Pneumococcal Growth Inhibition by Corynebacterium Strains

To isolate Corynebacterium strains from infant nasopharyngeal samples, 10 μL of sample were streaked on plates containing brain heart infusion (BHI) medium (Sigma-Aldrich) supplemented with 50 μg/mL of fosfomycin disodium (Fisher Scientific) and 1% Tween 80 (VWR). Single bacterial colonies were subcultured to 5% sheep blood agar plates (Fisher Scientific) and identified bacterial species using a VITEK MS automated mass spectrometry microbial identification system (bioMerieux). Cell-free growth inhibition assays were used to screen Corynebacterium strains for the secretion of anti-pneumococcal factors. Corynebacterium strains were grown in either 20 mL of BHI medium supplemented with 0.2% Tween 80 for 12-18 hours at 37° C. and 5% CO₂. These cultures were centrifuged at 3000 rpm for 10 minutes to generate cell pellets, and the supernatants were sterile-filtered with a 0.22-μM filter. The resulting cell-free media was diluted 50% in tryptic soy broth (TSB; Fisher Scientific) and glycerol stocks of two strains of S. pneumoniae, one a reference strain (ATCC 6303; serotype 3) and the second strain isolated from an infant nasopharyngeal sample (05-160; serotype 11A), were separately diluted 1:50 into the diluted cell-free media. Growth was assessed by OD600 readings relative to blank media controls for each growth medium over 24 hours.

### SPECIFIC EXAMPLES 1-10

### Example 1

### The Nasopharyngeal Microbiome was a Low-Diversity Microbial Community Throughout Infancy

Nasopharyngeal swab samples were collected monthly (0-6 months) or bimonthly (6-12 months) from 179 mother-infant dyads recruited at urban and rural study sites in southern Botswana. Infants were born vaginally, had a median [interquartile range (IQR)] birth weight of 3120 grams (2855 grams, 3408 grams), and were predominantly breastfed (Table 3).

TABLE 3
Characteristics of the 179 Mother-Infant Dyads
Included in the Study Population.

| Subject Characteristics | N | % |
|---|---|---|
| Infant sex | | |
| Female | 99 | 55% |
| Male | 80 | 45% |
| Median (IQR) infant birth weight, g | 3120 | (2855, 3408) |
| Infant HIV status | | |
| HIV-exposed, uninfected | 51 | 28% |
| HIV-unexposed, uninfected | 128 | 72% |
| Location of residence | | |
| Rural | 72 | 40% |
| Urban | 107 | 60% |
| Electricity in the home | 119 | 66% |
| Household use of solid fuels | 104 | 58% |
| Maternal educational level | | |
| None or primary | 13 | 7% |
| Secondary | 144 | 80% |
| Tertiary | 22 | 12% |
| Median (IQR) number of household members | | |
| <5 years | 1 | (0, 1) |
| 5-17 years | 1 | (0, 3) |
| ≥18 years | 3 | (2, 5) |
| Enrollment season | | |
| Summer (November to March) | 67 | 37% |
| Winter (April to October) | 112 | 63% |
| One or more PCV-13 doses | 153 | 85% |
| Infant breastfeeding, any | 153 | 85% |
| Infant antibiotic exposure, any | 114 | 64% |
| Amoxicillin | 83 | 46% |
| Metronidazole | 23 | 13% |
| Trimethoprim-sulfamethoxazole | 43 | 24% |

IQR (interquartile range) PCV-13 (13-valent pneumococcal conjugate vaccine)

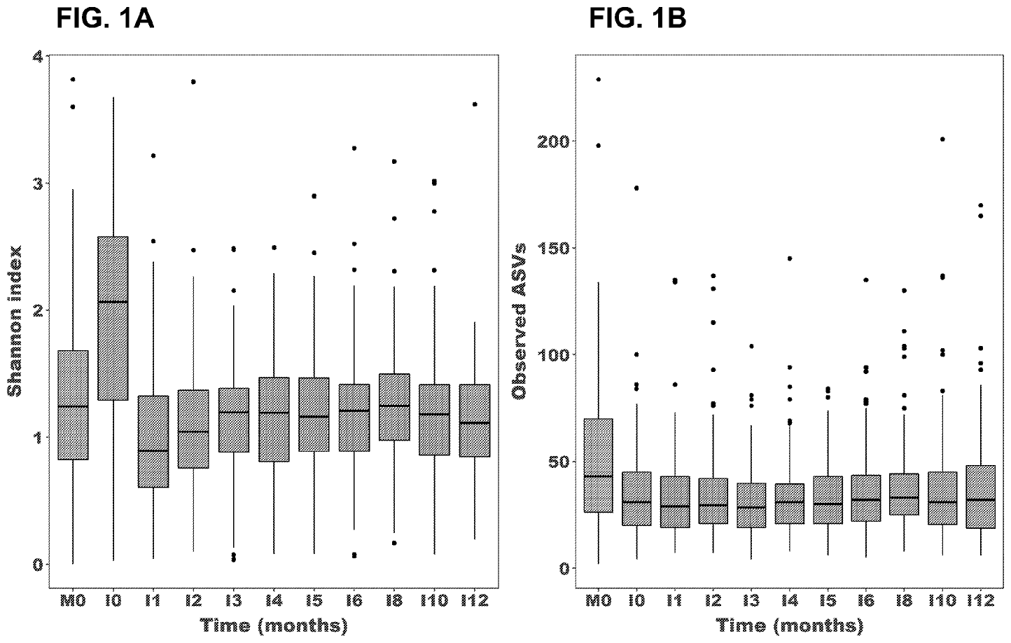

Infants were followed in this study to a median (IQR) age of 12.0 (8.0, 12.1) months. Of 51 mothers with HIV, 44 (86%) mothers received antiretroviral therapy during pregnancy for a median (IQR) duration of 9 months (5, 9); median (IQR) CD4 count was 461 cells/μL (312, 641). 16S rRNA sequencing of (i) infant nasopharyngeal samples was performed from all study visits, and (ii) maternal nasopharyngeal samples from the delivery visit. This resulted in 1368 infant and 172 maternal samples passing quality control procedures. The median (IQR) Shannon index and number of unique ASVs in these samples were 1.38 (1.03, 1.72) and 71 (50, 100), respectively. Infant nasopharyngeal microbiome diversity remained relatively stable during infancy and similar to maternal nasopharyngeal microbiome diversity (FIG. 1A-FIG. 1B), except at birth (I0), when diversity was higher than later in infancy (Wilcoxon signed-rank tests, p<0.0001) and compared to maternal samples (Wilcoxon signed-rank test, p<0.0001). Nasopharyngeal microbiome richness increased with age during infancy (negative binomial regression, p<0.0001) and did not differ from the richness of the maternal nasopharyngeal microbiome after five months of age (Wilcoxon signed-rank tests, p>0.05).

### Example 2

### Rapid Shifts in Nasopharyngeal Microbiome Composition Occurred During Early Infancy

Six bacterial genera—Corynebacterium, Dolosigranulum, Haemophilus, Moraxella, Staphylococcus, and Streptococcus—accounted for more than 90% of the sequencing reads identified in infant nasopharyngeal samples collected at or after one month of age (Table 4).

TABLE 4
Relative Abundances of Phyla and Highly Abundant Genera in Infant
Nasopharyngeal Samples at Birth and 1-12 Months of Age.

| Phylum/genus | Birth (n = 131) | Months 1-12 (n = 1,237) |
|---|---|---|
| Actinobacteriota | 12.5% | 22.4% |
| Corynebacterium | 4.6% | 19.5% |
| Gardnerella | 2.7% | <0.1% |
| Rothia | 0.2% | 0.1% |
| Bacteriodota | 10.5% | 0.7% |
| Alloprevotella | <0.1% | 0.2% |
| Prevotella | 3.2% | 0.2% |
| Fusobacteriota | 2.0% | 0.2% |
| Fusobacterium | 0.2% | 0.2% |
| Sneathia | 1.8% | <0.1% |
| Proteobacteria | 16.3% | 44.0% |
| Acinetobacter | 2.0% | 0.1% |
| Haemophilus | 1.3% | 8.9% |
| Moraxella | 1.6% | 29.6% |
| Neisseria | 0.2% | 0.3% |
| Pseudomonas | 0.6% | 0.4% |
| Firmicutes | 58.5% | 32.4% |
| Dolosigranulum | 1.3% | 14.3% |
| Gemella | 2.6% | 0.3% |
| Granulicatella | <0.1% | 0.1% |
| Lactobacillus | 4.1% | <0.1% |
| Megasphaera | 0.4% | <0.1% |
| Mycoplasma | 1.8% | <0.1% |
| Staphylococcus | 21.7% | 10.7% |
| Streptococcus | 1.7% | 10.4% |
| Veillonella | 0.3% | 0.2% |
| Other | 0.2% | 0.3% |

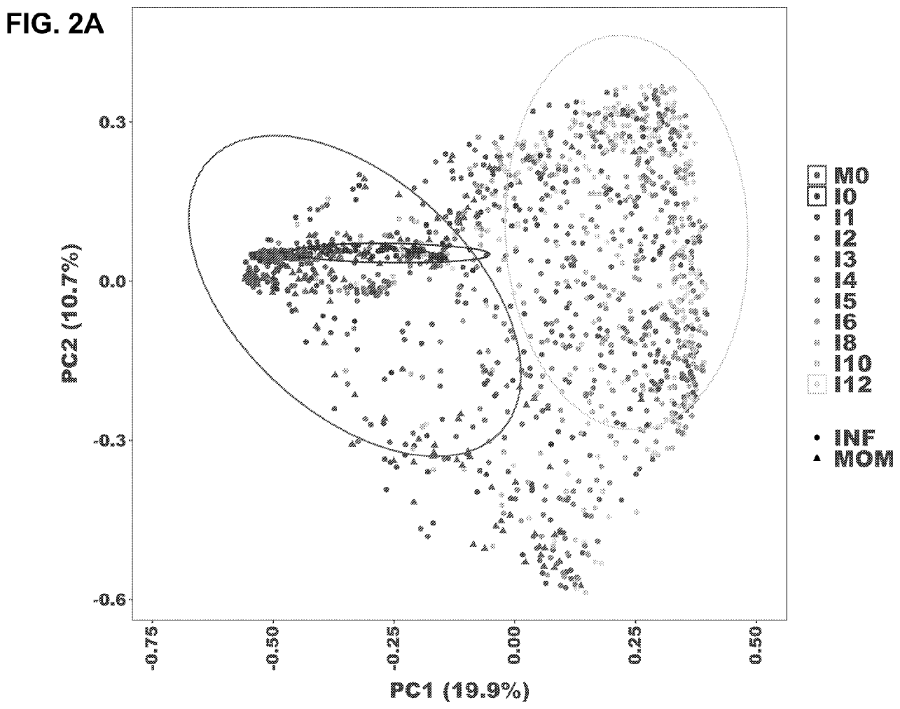

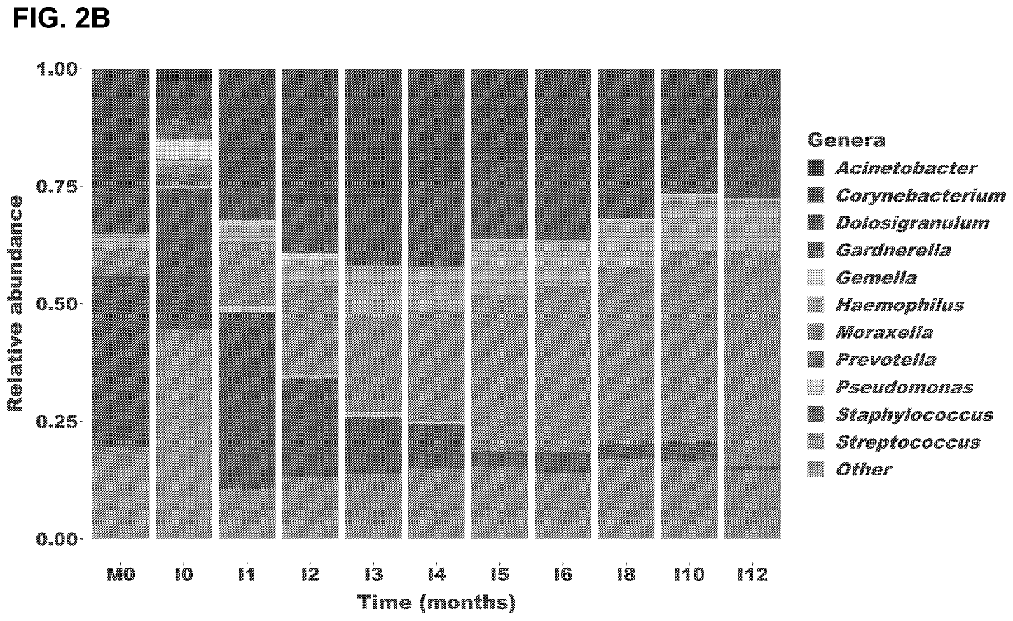

Only at the birth visit were other bacterial genera abundant, including Acinetobacter, Gardnerella, Lactobacillus, and Sneathia, likely reflecting colonization of the infant upper respiratory tract by pioneering microbes from the maternal gut (49, 50) and vaginal microbiomes (50, 51). Nasopharyngeal microbiome composition shifted dramatically during early infancy (FIG. 2A-FIG. 2B). Microbiome composition at each study visit differed from the preceding visit from birth through five months of age (PERMANOVA on Bray-Curtis dissimilarity, p<0.05), after which no significant differences in overall microbiome composition were observed between consecutive study visits. While maternal nasopharyngeal microbiome composition at the birth visit differed from infant microbiome composition at all study visits (PERMANOVA on Bray-Curtis dissimilarity, p<0.001), the dissimilarity of these microbiomes increased with age (linear mixed effects model, p<0.0001), indicating progressive divergence of the infant nasopharyngeal microbiome from an adult microbiome profile during the first 12 months of life.

To further describe shifts in microbiome composition during infancy, each infant nasopharyngeal sample was classified based upon whether a single genus comprised 5000 or more of the sequencing reads in that sample. Samples for which no genus met this relative abundance threshold were categorized as “biodiverse.” A single genus was dominant in 844 of 1368 (62) infant samples with biodiverse (n=524; 38%), Moraxella-dominant (n=343; 25), Corynebacterium-dominant (n=153; 11%), and Staphylococcus-dominant (n=142; 10) being the most common microbiome “biotypes” (Table 5).

TABLE 5
Prevalence of Specific Microbiome “Biotypes” in
1,368 Infant Nasopharyngeal Samples

| | Number | Sample |
|---|---|---|
| | of Samples | Prevalence (%) |
| Biodiverse | 524 | 38% |
| Dominated by a single genus | 844 | 62% |
| Corynebacterium | 153 | 11% |
| Dolosigranulum | 51 | 4% |
| Haemophilus | 85 | 6% |
| Moraxella | 343 | 25% |
| Staphylococcus | 142 | 10% |
| Streptococcus | 55 | 4% |
| Other genera* | 15 | 1% |

*Samples dominated by Pseudomonas (n = 4), Gemella (n = 2), Lactobacillus (n = 2), Mycoplasma (n = 2), Neisseria (n = 1), Acinetobacter (n = 1) Gardnerella (n = 1), Fusobacterium (n = 1), Sneathia (n = 1)

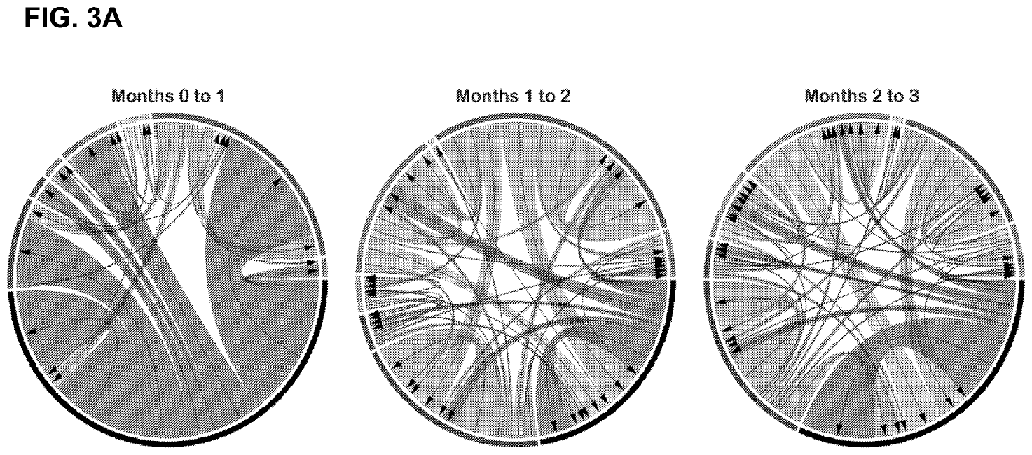

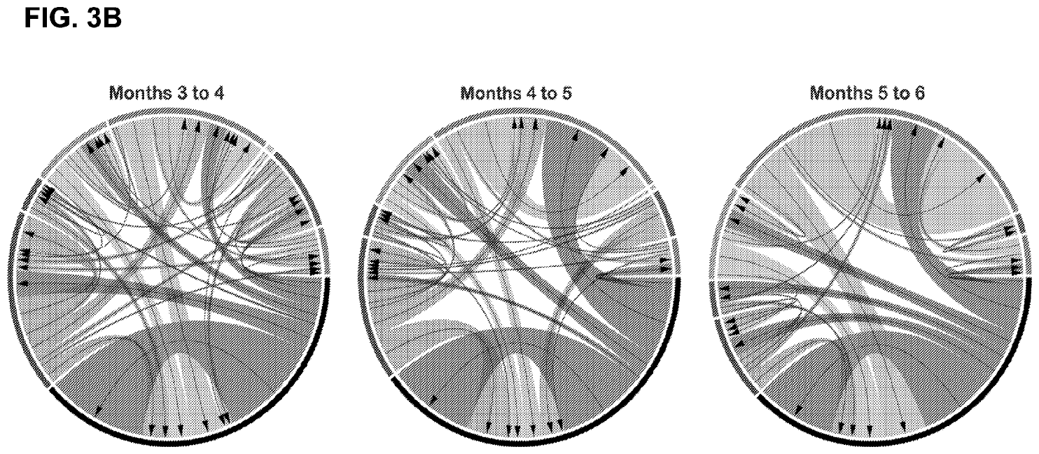

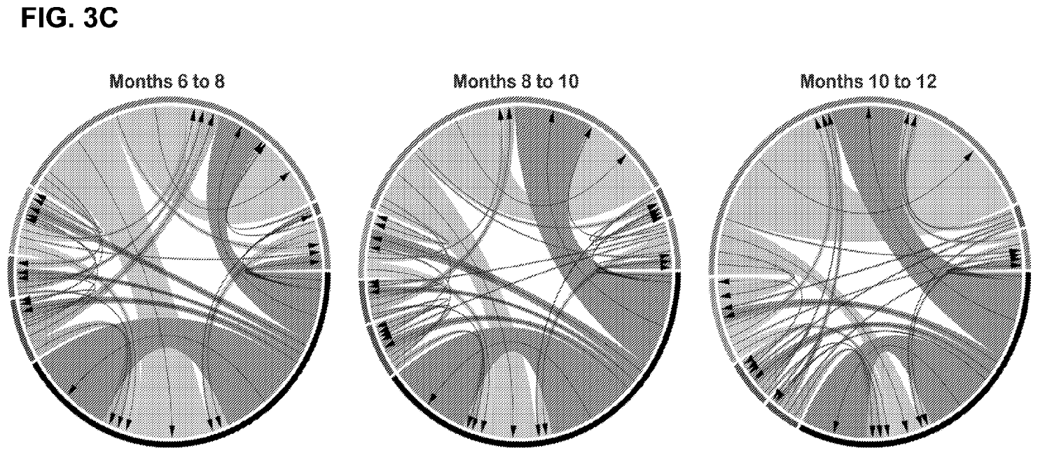

A per sample microbiome profile was considered “stable” when the next sample from that infant was classified as the same biotype. Based on this definition, within-infant stability of the nasopharyngeal microbiome varied by biotype (FIG. 3A-FIG. 3C and Table 6).

TABLE 6
Logistic Regression Model Analyses Evaluating the Stability of Specific
Nasopharyngeal Microbiome “Biotypes” During Infancy.

| | # of | Biotype | | Odds | | |
|---|---|---|---|---|---|---|
| | Samples | Transitions (N, %) | | Ratio | 95% CI | p |
| Microbiome biotype | | | | | | |
| Biodiverse | 471 | 283 | 60% | 1.00 | Ref | — |
| Dominated by a single genus | | | | | | |
| Corynebacterium | 144 | 97 | 67% | 1.34 | (0.91-2.00) | 0.15 |
| Dolosigranulum | 41 | 34 | 83% | 3.29 | (1.51-8.24) | 0.005 |
| Haemophilus | 73 | 58 | 79% | 2.66 | (1.50-5.00) | 0.001 |
| Moraxella | 275 | 142 | 52% | 0.75 | (0.55-1.02) | 0.07 |
| Staphylococcus | 134 | 94 | 70% | 1.45 | (0.95-2.22) | 0.08 |
| Streptococcus | 46 | 41 | 89% | 5.58 | (2.37-16.41) | 0.0003 |
| Other | 11 | 9 | 82% | 2.68 | (0.68-17.74) | 0.21 |

CI (confidence interval)
Analyses were adjusted for infant age and limited to samples for which nasopharyngeal microbiome data were available from the next study visit

Analyses were adjusted for infant age and limited to samples for which nasopharyngeal microbiome data were available from the next study visit. Compared to a biodiverse microbiome profile, a Moraxella-dominant biotype tended to be associated with higher stability (logistic regression model, p=0.07), while lower stability was observed with Dolosigranulum-dominant (p=0.005), Haemophilus-dominant (p=0.001), and Streptococcus-dominant (p=0.0003) biotypes.

### Example 3

### Environmental Exposures Influenced Nasopharyngeal Microbiome Composition During Infancy

To identify early life factors that influence the nasopharyngeal microbiome, MaAsLin2 (52) was used to fit generalized linear mixed models evaluating associations between sociodemographic factors and environmental exposures and the abundances of specific bacterial genera within the nasopharyngeal microbiome (Table 7).

TABLE 7
Significant Associations Between Sociodemographic
Factors and Environmental Exposures and the
Relative Abundances of Bacterial Genera from Generalized
Linear Mixed Models Implemented in MaAsLin2.

| | | Standard | |
|---|---|---|---|
| | Coefficient | Error | q |
| HIV-exposed, uninfected infant | | | |
| Acinetobacter | 1.14 | 0.37 | 0.12 |
| Household use of solid fuels | | | |
| Moraxella | 1.78 | 0.43 | 0.002 |
| Number of | | | |
| child household members | | | |
| Moraxella | 2.13 | 0.49 | 0.001 |
| Winter season | | | |
| Acinetobacter | −0.65 | 0.23 | 0.04 |
| Bergeyella | −0.66 | 0.22 | 0.03 |
| Corynebacterium | −0.60 | 0.18 | 0.02 |
| Haematobacter | −0.26 | 0.11 | 0.11 |
| Haemophilus | 1.04 | 0.29 | 0.01 |
| Janibacter | −0.31 | 0.13 | 0.10 |
| Microbacterium | −0.36 | 0.15 | 0.07 |
| Micrococcus | −0.57 | 0.16 | 0.01 |
| Stenotrophomonas | −0.49 | 0.17 | 0.04 |
| Number of PCV-13 doses | | | |
| Staphylococcus | −1.90 | 0.21 | <0.0001 |
| Dolosigranulum | 1.52 | 0.21 | <0.0001 |
| Moraxella | 1.50 | 0.22 | <0.0001 |
| Haemophilus | 0.99 | 0.26 | 0.002 |
| Lactobacillus | −0.65 | 0.19 | 0.008 |
| Streptococcus | 0.80 | 0.25 | 0.01 |
| Alloprevotella | 0.52 | 0.18 | 0.03 |
| Dermabacter | −0.43 | 0.15 | 0.04 |
| Anaerococcus | −0.42 | 0.16 | 0.05 |
| Butyribivibrio | −0.34 | 0.15 | 0.12 |
| Granulicatella | 0.41 | 0.18 | 0.12 |
| Stenotrophomonas | −0.34 | 0.16 | 0.17 |
| Finegoldia | −0.21 | 0.10 | 0.17 |
| Infant Breastfeeding | | | |
| Acinetobacter | −0.74 | 0.31 | 0.06 |
| Aggregatibacter | −0.26 | 0.14 | 0.16 |
| Alloprevotella | −0.65 | 0.23 | 0.02 |
| Anaerococcus | 0.42 | 0.21 | 0.15 |
| Corynebacterium | 0.85 | 0.24 | 0.003 |
| Dolosigranulum | −2.12 | 0.34 | <0.0001 |
| Fusobacterium | −0.47 | 0.20 | 0.07 |
| Granulicatella | −0.70 | 0.21 | 0.006 |
| Haemophilus | −2.15 | 0.40 | <0.0001 |
| Kluyvera | −0.53 | 0.23 | 0.07 |
| Leptotrichia | −0.67 | 0.13 | <0.0001 |
| Moraxella | −2.52 | 0.38 | <0.0001 |
| Mycoplasma | 0.44 | 0.23 | 0.16 |
| Neisseria | −0.65 | 0.24 | 0.03 |
| Porphyromonas | −0.82 | 0.24 | 0.005 |
| Staphylococcus | 2.48 | 0.38 | <0.0001 |
| Streptococcus | −0.92 | 0.35 | 0.03 |
| Receipt of antibiotics | | | |
| Actinomyces | −0.70 | 0.23 | 0.02 |
| Anaerococcus | −0.67 | 0.22 | 0.02 |
| Corynebacterium | −0.65 | 0.24 | 0.04 |
| Dolosigranulum | 1.12 | 0.33 | 0.007 |
| Haemophilus | 1.32 | 0.38 | 0.007 |
| Lactobacillus | −0.58 | 0.27 | 0.15 |
| Moraxella | 1.56 | 0.36 | 0.0004 |
| Peptoniphilus | −0.37 | 0.17 | 0.15 |
| Rothia | −0.45 | 0.22 | 0.15 |
| Staphylococcus | −1.96 | 0.36 | <0.0001 |
| Streptococcus | 0.94 | 0.35 | 0.04 |

In Table 7, positive (negative) coefficients correspond to relative increases (decreases) in genera relative abundances and q values correspond to false discovery rate-adjusted p values.

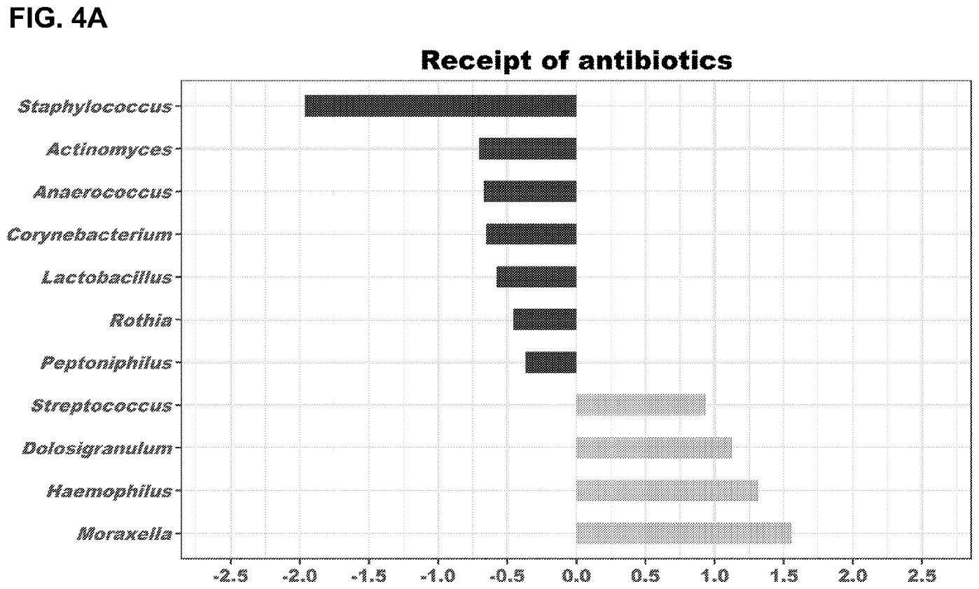

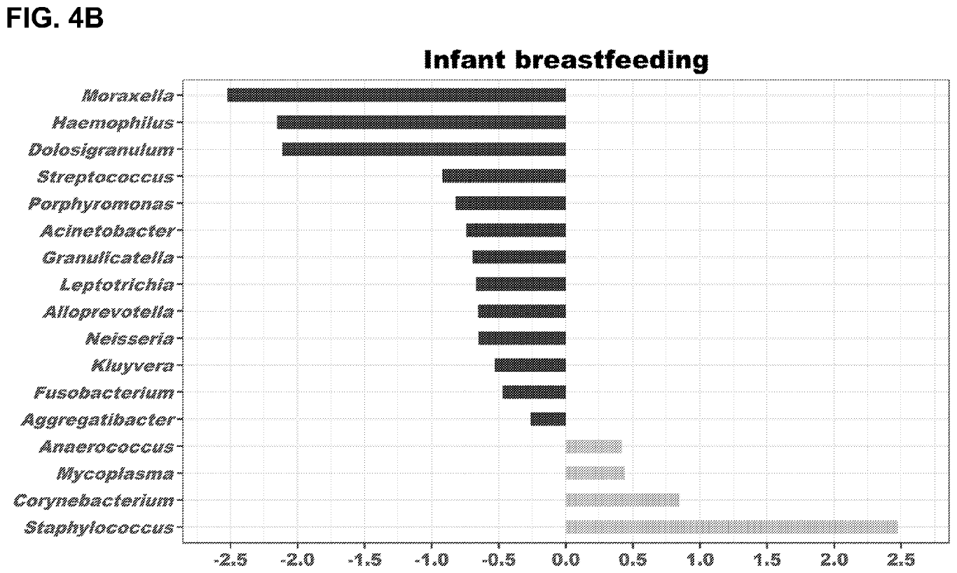

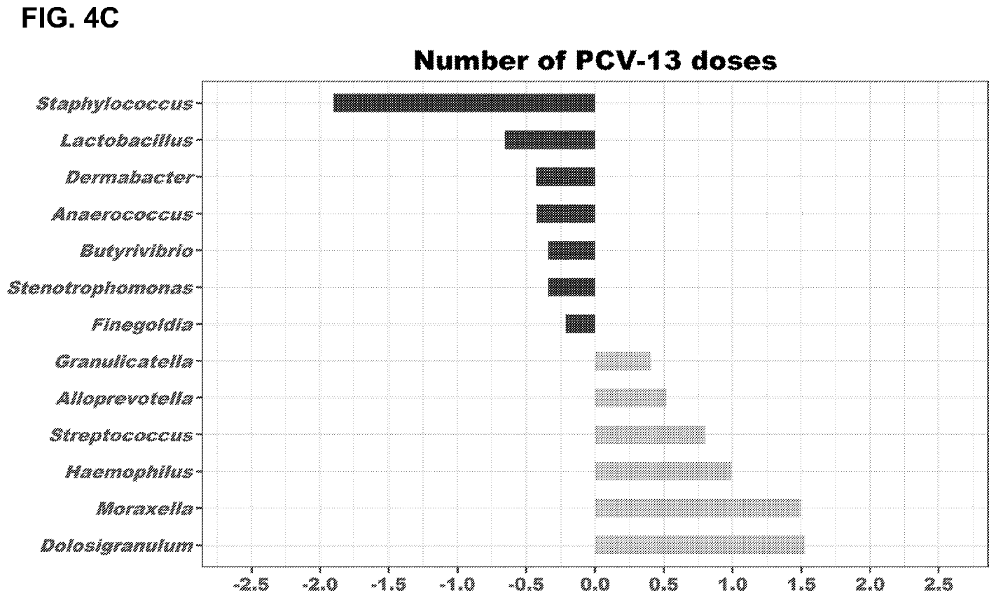

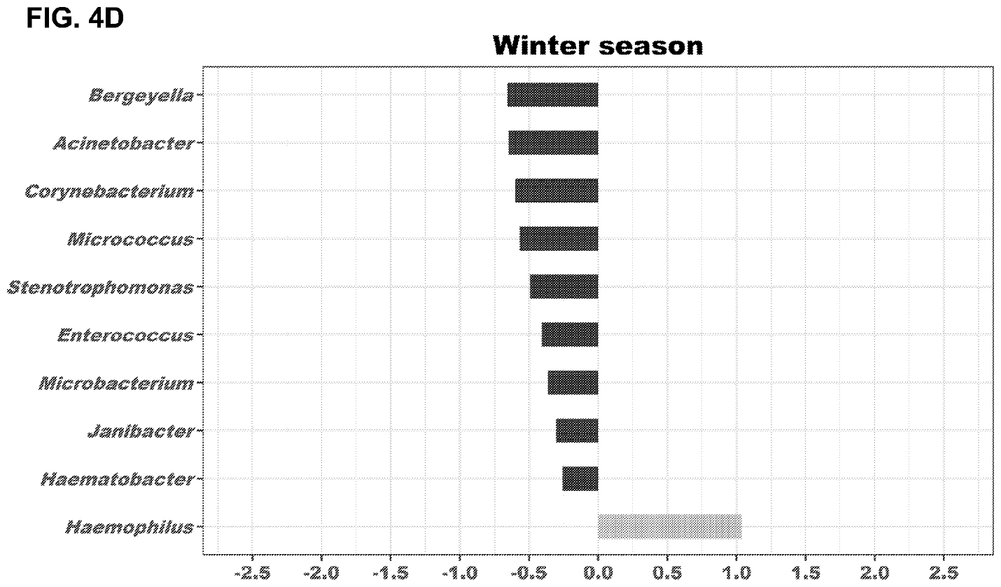

The most substantial microbiome composition changes were associated with recent antibiotic exposures, 13-valent pneumococcal conjugate vaccine (PCV-13) doses, breastfeeding, and the winter season (FIG. 4A-FIG. 4D). Antibiotic exposures were associated with decreases in the relative abundances of several bacterial genera generally associated with respiratory health, including Corynebacterium and Lactobacillus (31, 53-55), and increases in the relative abundances of genera containing common respiratory pathobionts (Haemophilus, Moraxella, Streptococcus). Similarly, during winter months, the relative abundance of Corynebacterium declined and was accompanied by an increase in the relative abundance of Haemophilus. In contrast, breastfeeding was associated with an increase in the relative abundance of Corynebacterium and decreases in the relative abundances of Haemophilus, Moraxella, and Streptococcus. No significant differences in nasopharyngeal microbiome composition were observed by sex, low birth weight status, and urban residence.

### Example 4

Corynebacterium Species were Associated with a Lower Risk of S. pneumoniae Colonization

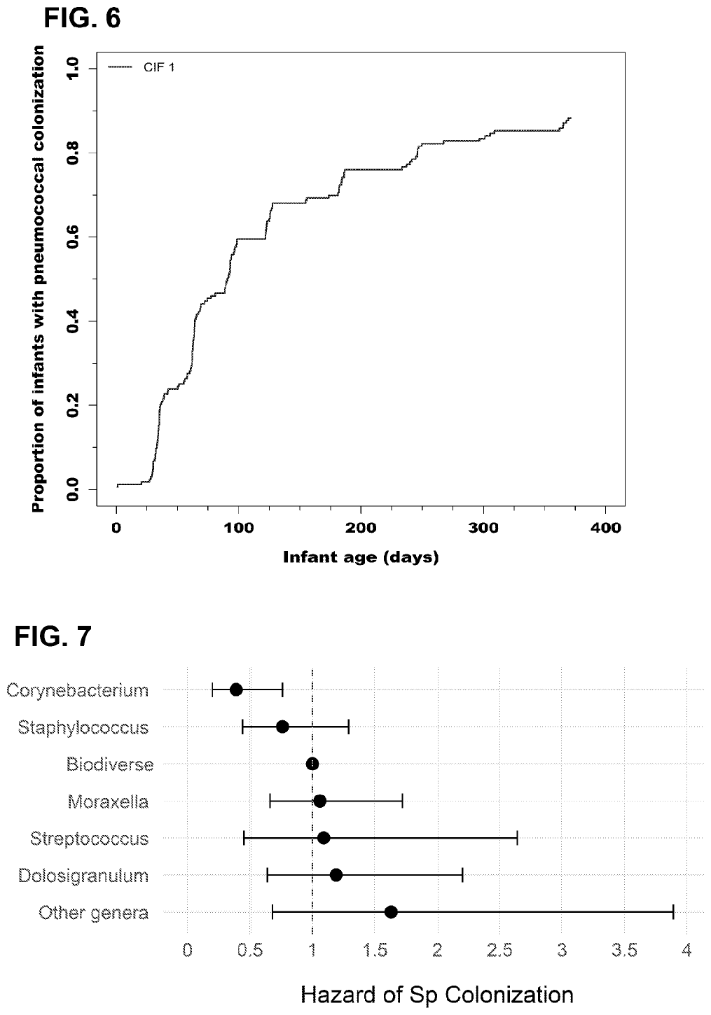

S. pneumoniae colonization was identified in 144 of 179 (80%) infants at a median (IQR) age of 71 (39, 126) days (FIG. 6). To identify nasopharyngeal microbiome features that precede S. pneumoniae colonization, a Cox proportional hazards model was first fit to evaluate associations between nasopharyngeal microbiome biotypes and acquisition of S. pneumoniae at the next study visit (Table 8).

TABLE 8
Cox Proportional Hazards Model Analyses Evaluating
Associations Between Microbiome “Biotypes”
Identified in Infant Nasopharyngeal Samples and the Risk
of Acquisition of S. pneumoniae Prior to the Next Study Visit.

| | Hazard | | |
|---|---|---|---|
| | Ratio | 95% CI | p |
| Microbiome biotype^{†} | | | |
| Biodiverse | 1.00 | Ref | — |
| Dominated by a single genus | | | |
| Corynebacterium | 0.43 | (0.23-0.80) | 0.008 |
| Dolosigranulum | 1.40 | (0.79-2.48) | 0.25 |
| Haemophilus | 1.32 | (0.49-3.54) | 0.58 |
| Moraxella | 1.15 | (0.73-1.82) | 0.55 |
| Staphylococcus | 0.70 | (0.39-1.25) | 0.23 |
| Streptococcus | 1.23 | (0.23-6.68) | 0.81 |
| Other genera | 3.34 | (1.28-8.70) | 0.01 |
| Male infant sex | 0.97 | (0.71-1.31) | 0.82 |
| Infant low birth weight (<2500 g) | 0.82 | (0.45-1.50) | 0.52 |
| HIV-exposed, uninfected infant | 1.11 | (0.70-1.76) | 0.65 |
| Urban residence | 1.35 | (0.98-1.86) | 0.07 |
| Household use of solid fuels | 1.12 | (0.80-1.57) | 0.51 |
| Number of child household members | 1.21 | (1.09-1.34) | 0.0003 |
| Winter season^{†} | 1.48 | (1.07-2.04) | 0.02 |
| Number of PCV-13 doses^{†} | 0.10 | (0.06-0.16) | <0.0001 |
| Infant breastfeeding^{†} | 0.99 | (0.63-1.55) | 0.97 |
| Receipt of antibiotics^{†} | 0.79 | (0.54-1.17) | 0.24 |

CI (confidence interval)
PCV-13 (13-valent pneumococcal conjugate vaccine)
^{†}Variable was modeled as a time-dependent covariate

Compared to a biodiverse microbiome profile, a Corynebacterium-dominant biotype was associated with a lower hazard of S. pneumoniae colonization [hazard ratio (THR): 0.43, 9500 confidence interval (CI): 0.23-0.80]. Additionally, the risk of S. pneumoniae colonization increased during winter months (HR: 1.48, 95% CI: 1.07-2.04) and with each additional child household member (THR: 1.21, 95% CI: 1.09-1.34), and declined with increasing number of PCV-13 doses (THR: 0.10, 95% CI: 0.06-0.16). To further explore the negative association between Corynebacterium species and S. pneumoniae, samples were classified into quartiles based on Corynebacterium relative abundance and evaluated the association between these sample quartiles and S. pneumoniae acquisition. The hazard ratio for pneumococcal colonization declined with each successive quartile increase in Corynebacterium relative abundance (Table 9); compared to the lowest quartile, the highest quartile of Corynebacterium relative abundance was associated with a 69% lower hazard of S. pneumoniae colonization (HR: 0.31, 95% CL 0.18-0.53).

TABLE 9
Cox Proportional Hazards Model Analyses Evaluating
the Association Between the Relative Abundance of
Corynebacterium in Infant Nasopharyngeal Samples
and the Risk of Acquisition of S. pneumoniae
Prior to the Next Study Visit.

| | Hazard | | |
|---|---|---|---|
| | Ratio | 95% C | p |
| Corynebacterium relative abundance* | | | |
| Quartile 1 (0.0%-3.1%) | 1.00 | Ref | — |
| Quartile 2 (3.1%-14.5%) | 0.87 | (0.56-1.36) | 0.55 |
| Quartile 3 (14.5%-40.6%) | 0.57 | (0.36-0.91) | 0.02 |
| Quartile 4 (40.6%-99.9%) | 0.31 | (0.18-0.53) | <0.0001 |
| Male infant sex | 1.07 | (0.73-1.58) | 0.72 |
| Infant low birth weight (<2500 g) | 0.55 | (0.22-1.41) | 0.21 |
| HIV-exposed, uninfected infant | 1.13 | (0.71-1.80) | 0.61 |
| Urban residence | 1.45 | (0.99-2.14) | 0.06 |
| Household use of solid fuels | 1.33 | (0.88-2.01) | 0.18 |
| Number of child household members | 1.13 | (1.01-1.25) | 0.03 |
| Winter season^{†} | 1.51 | (0.97-2.36) | 0.07 |
| Number of PCV-13 doses^{†} | 0.08 | (0.05-0.14) | <0.0001 |
| Infant breastfeeding^{†} | 1.17 | (0.70-1.98) | 0.54 |
| Receipt of antibiotics^{†} | 0.96 | (0.63-1.45) | 0.83 |

CI (confidence interval); PCV-13 (13-valent pneumococcal conjugate vaccine)
^{†}Variable was modeled as a time-dependent covariate

### Example 5

Identification of Corynebacterium Species Negatively Associated with S. pneumoniae Colonization

Given the diversity of Corynebacterium species isolated from the human upper respiratory tract (56-58), whether specific species accounted for the negative association with S. pneumoniae colonization. BLAST searches were used to identify the species or group of closely related species (supraspecies) corresponding to each of the 279 Corynebacterium amplicon sequence variants (ASVs) identified in infant nasopharyngeal samples (59).

Those ASVs were operationally classified into 45 unique Corynebacterium species or supraspecies (Table 10), the most abundant of which were C. pseudodiphtheriticum/propinquum (90.90 sample prevalence, 10.4% mean relative abundance), C. accolens/macginley (72.1 sample prevalence, 5.8% mean relative abundance), and C. tuberculostearicum (54.5% sample prevalence, 1.2% mean relative abundance).

TABLE 10
Prevalence and Relative Abundances of Specific Corynebacterium
species/Supraspecies in 1,368 Infant Nasopharyngeal Samples.

| | | Mean |
|---|---|---|
| | Sample | Relative |
| Species | Prevalence | Abundance |
| C. pseudodiphtheriticum/propinquum | 90.9% | 10.4% |
| C. accolens/macginleyi | 72.1% | 5.8% |
| C. tuberculostearicum | 54.5% | 1.2% |
| C. amycolatum/lactis | 19.0% | 0.08% |
| C. aurimucosum | 14.9% | 0.2% |
| C. coyleae | 9.9% | 0.03% |
| C. striatum/simulans | 8.9% | 0.2% |
| C. minutissimum/spheniscorum/singulare | 8.6% | 0.04% |
| C. appendicis/aquatimens | 8.0% | 0.009% |
| C. diphtheriae/vitaeruminis | 5.3% | 0.01% |
| C. kroppenstedtii | 4.8% | 0.005% |
| C. ihumii/pilbarense/mucifaciens | 4.5% | <0.001% |
| C. oculi/ciconiae | 3.5% | 0.01% |
| C. gottingense/imitans | 3.0% | 0.006% |
| C. confusum | 2.7% | <0.001% |
| C. jeikeium | 2.3% | 0.01% |
| C. lipophiloflavum | 2.3% | 0.004% |
| C. xerosis/frenyi | 2.3% | 0.003% |
| C. urealyticum | 2.3% | 0.003% |
| C. lowii/mastitidis | 1.9% | 0.008% |
| C. terpenotabidum/variabile/freiburgense | 1.7% | 0.002% |
| C. maris | 1.5% | 0.005% |
| C. dorum | 1.5% | 0.002% |
| C. jeddahense | 1.2% | 0.006% |
| C. matruchotii | 1.2% | 0.002% |
| C. pseudotuberculosis/ulcerans | 1.2% | <0.001% |
| C. thomssenii/sundsvallense | 0.9% | 0.005% |
| C. timonense | 0.9% | <0.001% |
| C. glutamicum/efficiens/faecale | 0.7% | 0.002% |
| C. humireducens | 0.7% | <0.001% |
| C. massiliense | 0.6% | 0.001% |
| C. mycetoides/riegelii | 0.6% | 0.004% |
| C. ammoniagenes/casei/stationis | 0.4% | 0.002% |
| C. bovis | 0.3% | 0.004% |
| C. suicordis | 0.4% | 0.002% |
| C. tuscaniense | 0.4% | 0.001% |
| C. falsenii | 0.3% | <0.001% |
| C. testudinoris | 0.2% | 0.001% |
| C. glaucum | 0.2% | <0.001% |
| C. camporealensis/canis/epidermidicanis | 0.2% | <0.001% |
| C. glucuronolyticum | 0.2% | <0.001% |
| C. glyciniphilum | 0.1% | <0.001% |
| C. marinum | 0.1% | <0.001% |
| C. canus/halotolerans/nuruki | 0.07% | <0.001% |
| C. resistens/auriscanis | 0.07% | <0.001% |
| Unable to classify to species/ | 41.4% | 0.08% |
| supraspecies level | | |

Subsequent analyses demonstrated that the relative abundances of each of these three Corynebacterium species or supraspecies were inversely associated with the risk of S. pneumoniae colonization (Table 11). These findings illustrate the enormous diversity of Corynebacterium species that colonize the human upper respiratory tract and indicate that multiple Corynebacterium species likely contribute to colonization resistance to S. pneumoniae.

TABLE 11
Summary of Results from Cox Proportional Hazards Model
Evaluating the Association Between Each 10% Increase
in the Relative Abundance of the Three Most
Abundant Corynebacterium species or Supraspecies in
Infant Nasopharyngeal Samples and the Risk of
Acquisition of S. pneumoniae Prior to the Next Study Visit.

| | Hazard | | |
|---|---|---|---|
| | Ratio | 95% CI | p |
| C. accolens/macginleyi relative | 0.84 | (0.74-0.96) | 0.005 |
| abundance^{†} | | | |
| C. pseudodiphtheriticum/propinquum | 0.85 | (0.75-0.95) | 0.01 |
| relative abundance^{†} | | | |
| C. tuberculostearicum relative | 0.71 | (0.52-0.96) | 0.03 |
| abundance^{†} | | | |
| Male infant sex | 1.03 | (0.69-1.53) | 0.88 |
| Infant low birth weight (<2500 g) | 0.54 | (0.22-1.36) | 0.19 |
| HIV-exposed, uninfected infant | 1.11 | (0.69-1.78) | 0.66 |
| Urban residence | 1.47 | (0.996-2.16) | 0.053 |
| Household use of solid fuels | 1.31 | (0.86-2.00) | 0.21 |
| Number of child household members | 1.12 | (1.00-1.24) | 0.04 |
| Winter season^{†} | 1.47 | (0.94-2.29) | 0.09 |
| Number of PCV-13 doses^{†} | 0.08 | (0.05-0.14) | <0.0001 |
| Infant breastfeeding^{†} | 1.19 | (0.71-1.99) | 0.51 |
| Receipt of antibiotics^{†} | 1.01 | (0.67-1.51) | 0.96 |

CI (confidence interval); PCV-13 (13-valent pneumococcal conjugate vaccine)
^{†}Variable was modeled as a time-dependent covariate

### Example 6

### Strain-Specific Secretion of Antipneumococcal Factors by Corynebacterium Species

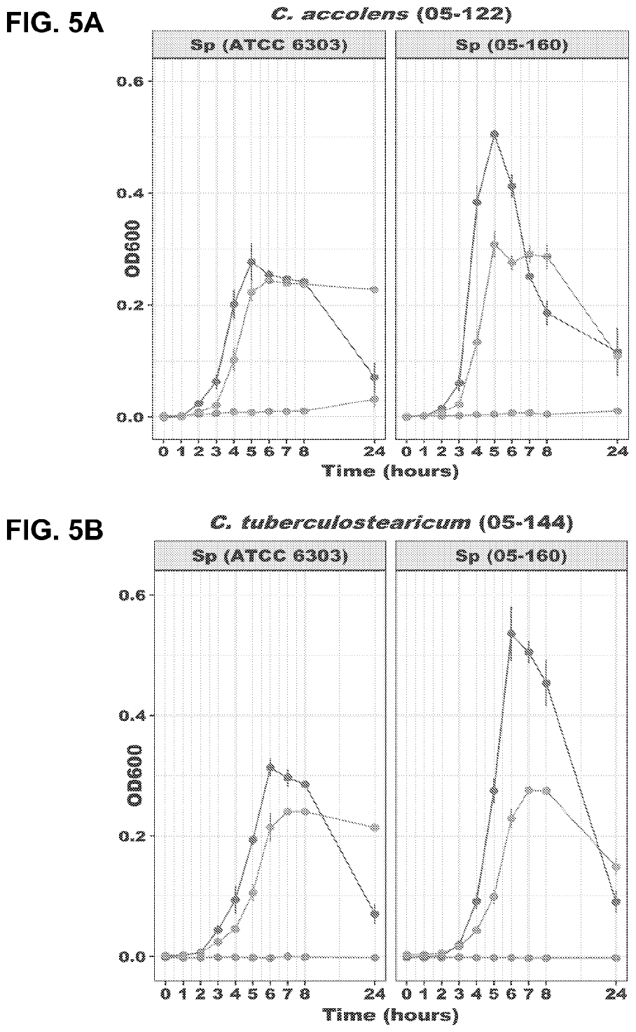

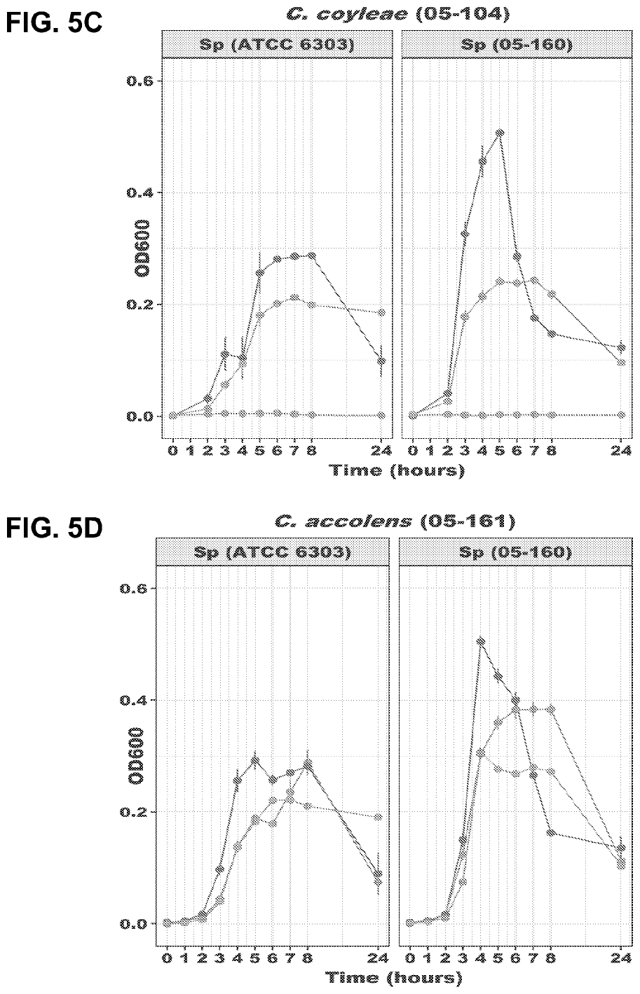

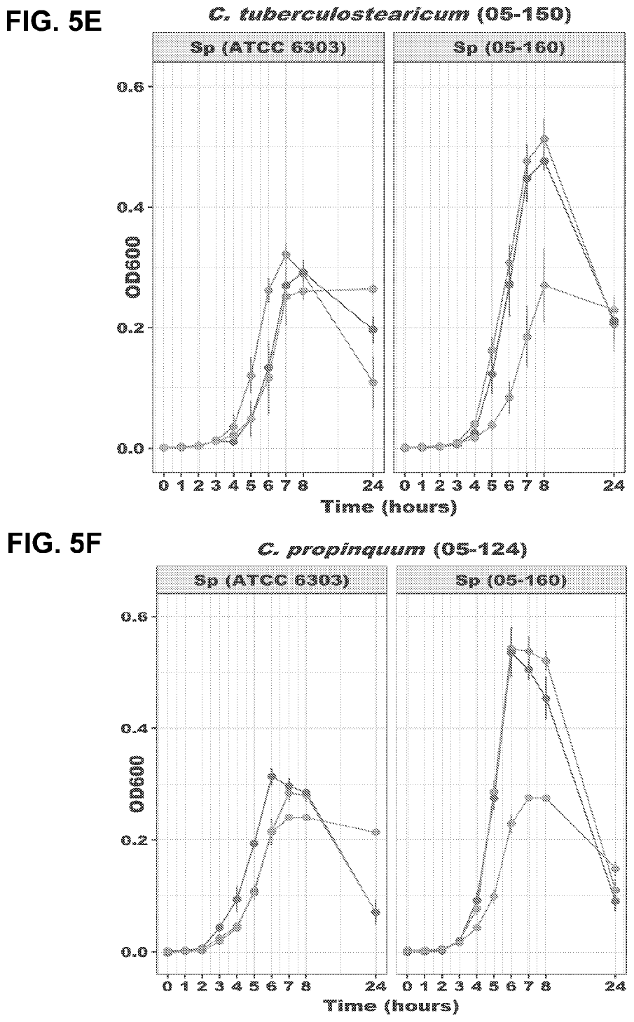

To further investigate the inhibition of S. pneumoniae by Corynebacterium species, infant nasopharyngeal samples were cultured on selective media and identified culture isolates to the species level using matrix-assisted laser desorption ionization time-of-flight mass spectrometry (MALDI-TOF MS). A total of 35 morphologically distinct Corynebacterium strains were isolated from 21 infants, including strains of C. accolens (n=21), C. tuberculostearicum (n=5), C. pseudodiphtheriticum (n=4), C. coyleae (n=2), C. propinquum (n=2), and C. striatum (n=1). The supernatants from cell cultures of each of these Corynebacterium strains were then screened for inhibition of growth of reference (ATCC 6303; serotype 3) and contemporary infant nasopharyngeal strains (05-160; serotype 11A) of S. pneumoniae. Inhibition of pneumococcal growth was observed by cell-free supernatants from cultures of seven (20%) strains FIG. 5A-FIG. 5C), including species accolens (1 of 21; 5%), tuberculostearicum (3 of 5; 60%), coyleae (2 of 2; 100%), and striatum (1 of 1; 100%). While the characteristics and mechanisms underlying the inhibition require further investigation, these assays support a causal basis for the inverse relationship between Corynebacterium spp. and S. pneumoniae observed in microbiome data analyses. Furthermore, these data indicate that specific species and strains have the capacity to inhibit S. pneumoniae.

### Summary of Examples 1-6

In this study, the nasopharyngeal microbiome dynamics during the first year of life were described and the environmental factors that influence infant nasopharyngeal microbiome composition were identified. Higher abundances of Corynebacterium species in the nasopharyngeal microbiome were associated with a lower risk of S. pneumoniae colonization during infancy. Finally, secreted factors from strains of several Corynebacterium species inhibited pneumococcal growth. The composition of the nasopharyngeal microbiome of infants in Botswana bears similarities to that reported in previous studies of infants and young children in high-income countries (39, 41). Specifically, independent of the resource setting, this low-diversity microbial community was typically comprised mostly of bacteria from six genera, with Corynebacterium and Staphylococcus predominating during the first several months of life and Dolosigranulum and Moraxella becoming more abundant later in infancy (Teo S M, et al. (2015) Cell Host Microbe. 17(5):704-715; Mika M, et al. (2015) J Allergy Clin Immunol. 135(4):905-912. e11). These trends in nasopharyngeal microbiome composition during infancy appeared to be highly conserved despite substantial differences in host characteristics, household exposures, and climate. However, some caution must be taken in these statements given that most studies of the infant nasopharyngeal microbiome have used 16S rRNA gene sequencing. Substantial functional or genomic differences may exist between the bacterial strains that colonize the upper respiratory tracts of children in geographically distinct human populations that would only be revealed with alternative methodologies.

The nasopharyngeal microbiome of infants in Botswana was found to be highly dynamic, although microbiome stability varied markedly based on the dominant genus. In particular, higher microbiome stability was observed with nasopharyngeal microbiome profiles that were dominated by Moraxella, while lower stability was seen with profiles that were dominated by Dolosigranulum, Haemophilus, or Streptococcus. Findings from studies conducted in high-income countries were broadly similar, although notably nasopharyngeal microbiome profiles with high abundance of Dolosigranulum were reported to be highly stable in these settings (39, 41). Further research is needed to investigate associations between the presence and abundance of Dolosigranulum in the nasopharyngeal microbiome and child respiratory health in low- and middle-income countries.

These findings also demonstrated the impact of early life environmental exposures on the nasopharyngeal microbiome. In particular, the composition of the nasopharyngeal microbiome of infants varied with season with winter months associated with a declining relative abundance of Corynebacterium. The association between season and the abundance of Corynebacterium in the nasopharyngeal microbiome could contribute to the higher incidence of S. pneumoniae colonization during the winter season observed in the cohort and reported in several prior studies (60-63). In contrast, a few changes in the infant nasopharyngeal microbiome associated with maternal HIV infection were found, indicating that other factors may account for the increased risks of pneumococcal colonization and disease observed in HIV-exposed, uninfected infants (64-66). Alternatively, the mild immunosuppression of mothers with HIV in this study may have limited the effect of HIV infection on the maternal upper respiratory microbiome; indeed, mothers with and without HIV in the cohort had similar nasopharyngeal microbiome composition (PERMANOVA on Bray-Curtis dissimilarity, p=0.98).

Feeding practices also influenced the composition of the nasopharyngeal microbiome of infants in this study, with breastfeeding promoting the enrichment of the nasopharyngeal microbiome with Corynebacterium species.

Interestingly, previous studies have not reported an association between breastfeeding and pneumococcal colonization during infancy (6, 67), which could reflect the complexity of the effect of breastfeeding on the infant upper respiratory microbiome.

Taken together, these findings indicate that the nasopharyngeal microbiome may be previously unrecognized and potentially modifiable mechanism by which environmental factors influence the risk of pneumococcal infections during childhood.

Higher abundances of Corynebacterium species within the nasopharyngeal microbiome are associated with a lower risk of S. pneumoniae colonization during infancy.

Strains of multiple Corynebacterium species that secrete factors that inhibit pneumococcal growth in laboratory experiments were identified. Corynebacterium is a diverse bacterial genus that includes common residents of the upper respiratory tracts, skin, and gastrointestinal tracts of humans and animals. Although more than 150 species of Corynebacterium have been identified to date (68), the most common species isolated from the human respiratory tract are C. accolens, C. pseudodiphtheriticum, C. propinquum, C. striatum, and C. tuberculostearicum (56-58). Except for Corynebacterium diphtheriae, the etiological agent of diphtheria (69), Corynebacterium species only rarely cause human disease, often in the setting of compromised host immunity (70, 71), indwelling prosthetic material (71-73), or chronic respiratory diseases (Renom F, et al. (2014) New Microbes New Infect. 2(4):106-114; Bittar F, et al. (2010) Emerg Infect Dis. 16(8):1231). This low pathogenicity of non-diphtheriae Corynebacterium species is a key characteristic that supports their further evaluation for use as biotherapeutics.

Despite the commonality of these species in the human microbiome, surprisingly little is known about the mechanisms by which Corynebacterium species adhere to human mucosal surfaces, interact with co-occurring microbial species and the host immune system, and rarely cause invasive infection.

Prior studies demonstrated antagonistic relationships between Corynebacterium species and several important bacterial pathobionts. C. propinquum was recently recognized to produce siderophores that inhibit the growth of coagulase-negative staphylococci through iron restriction (76), a strategy that this species may use to colonize the upper respiratory tract. Intranasal administration of a C. pseudodiphtheriticum strain effectively eradicated S. aureus nasal carriage in adults (77), demonstrating the potential use of Corynebacterium species as respiratory probiotics. Strains of C. pseudodiphtheriticum also inhibited growth of M. catarrhalis in co-cultivation experiments (78). Lower relative abundances of Corynebacterium species were previously observed in the upper respiratory tracts of children with S. pneumoniae colonization (6, 32, 33). Moreover, Bomar et al. identified a C. accolens strain that inhibited S. pneumoniae through the production of a lipase that releases antipneumococcal free fatty acids from human skin triacylglycerols (33). These findings demonstrate the substantial strain-level heterogeneity that characterizes interactions between Corynebacterium spp. and S. pneumoniae and point towards the need to study large collections of Corynebacterium strains to understand the breadth and distribution of phenotypes exhibiting anti-pneumococcal activity.

Comparative analyses of microbiome composition were based on sample relative abundances, and it was not possible to determine if the observed associations represent differences in the absolute amounts of bacterial genera or species within the nasopharynx across samples. This work employed 16S rRNA gene sequencing to characterize the nasopharyngeal microbiome of infants and mothers.

In summary, environmental exposures that shaped the developing infant nasopharyngeal microbiome were identified. These environmental exposures can influence colonization resistance to S. pneumoniae. Moreover, an inverse relationship between the abundances of Corynebacterium species within the nasopharyngeal microbiome and the risk of S. pneumoniae colonization during infancy was identified.

### Example 7

Higher NP Microbiota Corynebacterium Spp. Abundances Correlated with a Decreased Risk of Sp Colonization

Serial NP samples were from 300 infants (≤12 months of age) enrolled in a prospective cohort study in Botswana. Sp colonized more than half (54%) of infants by 2 months of age—the age for the first dose of 13-valent pneumococcal conjugate vaccine (PCV-13)—and Sp colonized more than 85% by age 12 months. Using 16S rDNA V4 region sequencing of NP samples, NP samples were classified by dominance with ≥50% of the nasopharyngeal microbiota comprised of a single genus. A single genus dominated 72% of samples with the most frequent “biotypes” being Moraxella-dominant (33%), biodiverse (72%), and Corynebacterium-dominant (25%). Sp colonized 11% (10 of 95) of infants with a Corynebacterium-dominant biotype. In contrast, Sp colonized 24-45% of infants with other biotypes. In multivariable analyses, the risk of Sp colonization decreased with a Corynebacterium-dominant biotype (FIG. 7). Of NP samples categorized into quartiles based on the Corynebacterium relative abundance, each successive quartile of Corynebacterium relative abundance correlated with a reduction in the risk of Sp colonization (Table 12).

TABLE 12
Higher Corynebacterium Relative Abundance
Associates with Lower Sp Colonization.

| | Sp colonization risk | |
|---|---|---|
| Corynebacterium abundance | HR | (95% CI) |
| 1^{st} quartile (lowest) | 1.00 | Ref |
| 2^{nd} quartile | 0.82 | (0.52−1.30) |
| 3^{rd} quartile | 0.65 | (0.40−1.04) |
| 4^{th} quartile (highest) | 0.33 | (0.20−0.56) |

HR, hazard ratio; CI, confidence interval; Ref, reference

These data demonstrated a strong inverse relationship between Corynebacterium spp. and Sp colonization in the human infant NP.

### Example 8

Respiratory Tract Corynebacterium Strains More Strongly Adhered to Respiratory Epithelium than Sp.

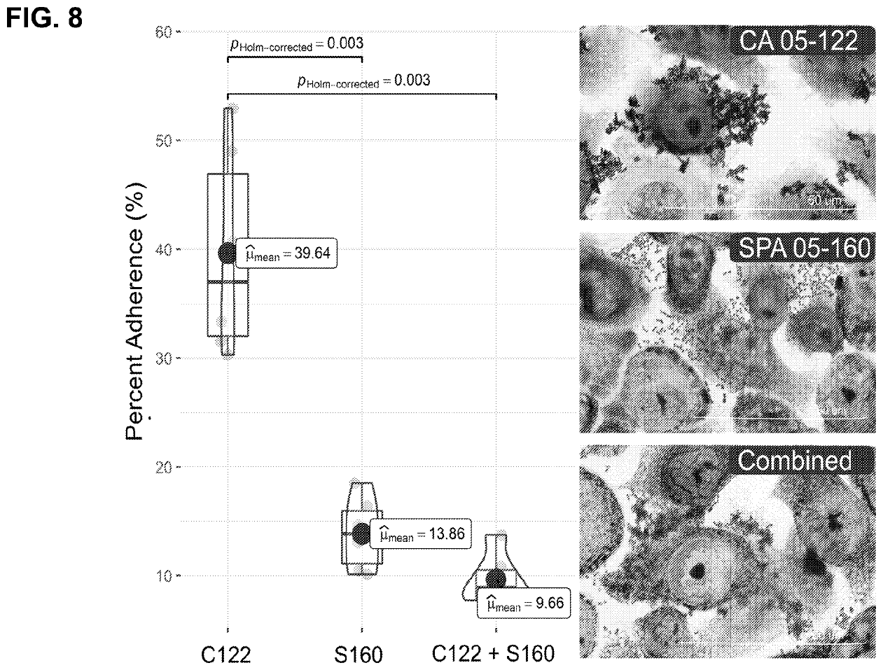

Several Corynebacterium spp were tested for adherence on the model respiratory cell line A549 (FIG. 8). C. accolens strain 05-122 demonstrated a median percent adherence of 39% at an MOI of 20:1 (bacteria to epithelial cells) with saturation of adherence between 20:1 and 40:1. Strains of C. coyleae (n=2) and C. pseudotuberculosis (n=2) similarly associated with the epithelial cells producing over 30% adherence. In contrast, Sp strains 05-160 (FIG. 8), and ATCC 6303 demonstrated less than 15% adherence. By microscopy, C. accolens demonstrated aggregated adherence to the monolayer, while Sp demonstrated more diffuse adherence with characteristic pairs. This work indicated a trend towards lower Sp adherence to epithelial previously colonized by C. accolens.

### Example 9

### Corynebacterium Strains Secreted Anti-Pneumococcal Factors

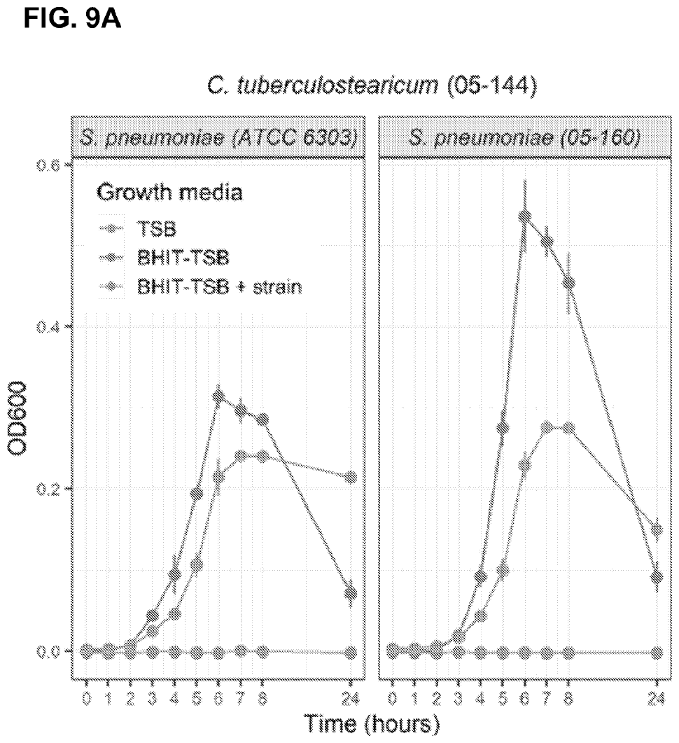

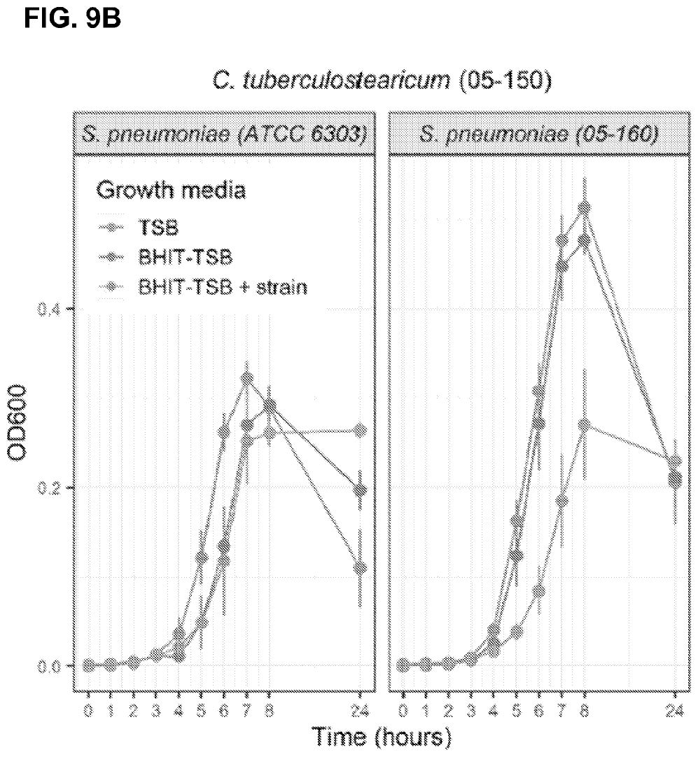

35 Corynebacterium human NP strains were selected for these experiments. Supernatants from Corynebacterium cell cultures were incubated with reference Sp strain (ATCC 6303; serotype 3) and infant NP Sp strain 05-160, serotype 11A. Inhibition of pneumococcal growth by cell-free supernatants from 7 (20%) strains, including species accolens (1 of 21; 5%), tuberculostearicum (3 of 5; 60%; FIG. 9A-FIG. 9B), coyleae (2 of 2; 100%), and striatum (1 of 1; 100%). coyleae strains produced secreted Sp inhibition without an added triacylglycerol source, making the previously described lipase conversion to Sp-inhibitory fatty acid mechanisms unlikely. (Bomar L, et al. (20165) MBio. 7(1):e01725-e1815). These experiments demonstrated that the inhibitory activity in the presence and absence of triacylglycerol sources was proteinaceous: (i) the activity was retained by 50 kDa ultrafiltration, and (ii) the activity for an initial tested accolens strain was abrogated by proteinase K treatment. These data indicated species- and strain-dependent secreted Sp inhibition mediated by multiple mechanisms.

### Example 10

### Generation of a Corynebacterium Pan-Genome

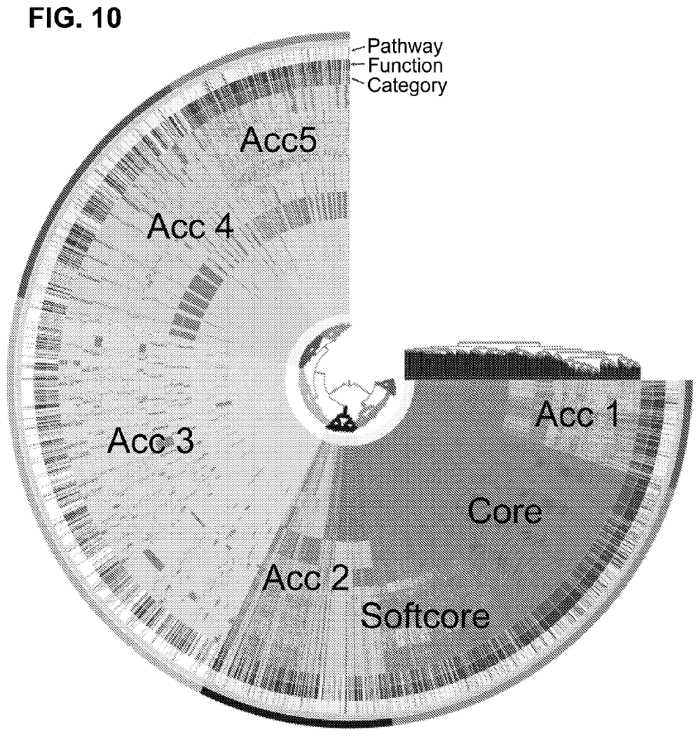

A preliminary pan-genome for the Corynebacterium genus was constructed using publicly available complete and draft genomes from the National Center for Biotechnology Information's GenBank database (FIG. 10). The genomes were re-annotated using Prodigal to identify open reading frames, tRNA, and ribosomal rDNA using a common approach to each genome. All vs. all clustering was performed using GET_HOMOLOGUES by blastp and OrthoMCL (inflation=2) with gene links made for E scores 1×10⁻³⁰ and ≥70% of the length of the shortest peptide in the group. (Contreras-Moreira B, et al. (2013) Appl Environ Microbiol. 79(24):7696-7701; Mahram A, et al. (2010) Proceedings of the 24th ACM International Conference on Supercomputing; Chen F, et al. (2006) Nucleic Acids Res. 34 (Database issue):D363-368). Small core and softcore gene sets were identified with genomes clustering by five groups of accessory genes (Acc 1-5; FIG. 10). While the preliminary pangenome was constructed from 273 complete genomes, a larger pangenome is constructed from a more extensive set of public data, including complete and draft genomes that meet the previously described criteria in addition to the high-quality genomes constructed as part of the proposed comparative genomics.

These data support the microbiome surveys described herein indicating that Corynebacterium spp. affect population-level trends in Sp colonization. These data also support the ability of Corynebacteria to directly inhibit Sp through multiple mechanisms, including secreted inhibitors and adherence. The data reinforce the rationale for using comparative genomics, model interspecies competition assays, and proteomics to identify Corynebacterium spp. mechanisms to exclude respiratory pathobiont during the rational design and selection of probiotics to prevent and treat respiratory infections.

### SPECIFIC EXAMPLES 11-12

Pneumonia leads among infectious causes of death among children, accounting for more than 800,000 deaths in 2017. Bacterial pathogens, particularly S. pneumoniae (Sp), associate with the highest mortality. Pneumococcal conjugate vaccines effectively prevent invasive diseases like meningitis and bacteremia caused by vaccine serotypes. (Waight P A, et al. (2015) Lancet Infect Dis. 15(6):629; Moore M R, et al. (2015) Lancet Infect Dis. 15(3):301-309). However, these vaccines are less effective in preventing Sp pneumoniae and other respiratory infections. (Cutts F T, et al. (2005) Lancet. 365(9465):1139-1146; Klugman K P, et al. (2003) N Engl J Med. 349(14):1341-1348; O'Brien K L, et al. (2009) Lancet. 374(9693):893-902; Horácio A N, et al. (2018) PLoS One. 13(11):e0206912). Moreover, vaccination yields an increase in disease caused by non-vaccine serotypes and less effective vaccine programs. (Dagan R, et al. (1996) J Infect Dis. 174(6):1271-1278; Hammitt L L, et al. (2014) Lancet Glob Health. 2(7):e397-405; Ladhani S N, et al. (2018) Lancet Infect Dis. 18(4):441-451; Burgos J, et al. (2013) Clin Microbiol Infect. 19(4):385-391; Kim S H, et al. (2020) Vaccine. 38(38):6065-6073; Park D C, et al. (2019) Ann Lab Med. 39(6):537-544; Pick H, et al. (2020) Thorax. 75(1):38-49).

As discussed above, pneumonia is the leading infectious killer of children, accounting for more than 800,000 child deaths each year. Streptococcus pneumoniae (Sp) causes most fatal cases of childhood pneumonia despite vaccination. Colonization of the nasopharynx (NP) precedes infections by Sp, and the NP microbiota serves as a barrier to pathogen colonization. In particular, recent studies indicate that Corynebacterium species provide colonization resistance to bacterial respiratory pathogens. There is a strong inverse relationship between the relative abundance of certain Corynebacterium spp. in the NP microbiota and the risk of Sp colonization among children. Further, studies detailed herein show specific Corynebacterium strains directly inhibited S. pneumoniae in vitro. These data, combined with the low pathogenicity of non-diphtheriae Corynebacterium spp., make these strains promising therapeutic candidates for pneumonia prevention. However, the mechanisms by which non-diphtheriae Corynebacteria adhere to human respiratory epithelium, inhibit respiratory pathogen colonization, and (rarely) cause invasive infection are not comprehensively understood. Understanding the relationship between Corynebacterium genetics and interspecies interactions provides critical knowledge about respiratory and invasive infection prevention and guides the development of Corynebacteria as biotherapeutics. To bridge this gap, in vitro interspecies interaction experiments, comparative genomics, and comparative proteomics are all conducted to elucidate the mechanisms by which Corynebacterium spp. interact with Sp in the human respiratory tract.

Prevention and treatment of respiratory and invasive infections is achieved through targeted manipulation of the microbiota. The identification of the mechanisms by which Corynebacterium spp. colonize the human NP and exclude or control Sp in this ecological niche. Respiratory strains of Corynebacterium adapted to prevent Sp colonization through competitive adherence and non-adherence mechanisms (e.g., secretion of antimicrobial substances, stimulation of release of antimicrobial factors from the respiratory epithelium) can be harnessed in developing effective biotherapeutics.

Here, 250 contemporary human respiratory and skin Corynebacterium strains collected from diverse patient populations are used to employ comparative genomics of corynebacteria to identify genotype-phenotype relationships between respiratory epithelium competitive adherence and Sp inhibition as detailed below.

### The Nasopharyngeal (NP) Microbiota Provides a Barrier to Colonization and Invasion by Bacterial Pathogens.

Colonization of the NP precedes pneumonia caused by Sp and other bacterial respiratory pathogens. (Bogaert D, et al. (2004) Lancet Infect Dis. (3):144-154; Wolter N, et al. (2014) J Infect Dis. 210(10):1649-1657; Garcia-Rodriguez J A, et al. (2002) J Antimicrob Chemother. 50 Suppl S2:59-73). Therefore, interventions that prevent NP colonization by these bacteria would be anticipated to lower pneumonia risk. Although more extensively reported in the intestinal and genitourinary tracts, the NP microbiota can also influence the risk of colonization by exogenous bacterial pathogens. (Chang J Y, et al. (2008) J Infect Dis. 197(3):435-438; van der Veer C, et al. (2017) Clin Infect Dis. 64(1):24-31; Ducluzeau R, et al. (1976) Antimicrob Agents Chemother. 9(1):20-25; Brotman R M, et al. (2010) J Infect Dis. 202(12):1907-1915). However, most studies of bacterial dynamics within the upper respiratory tract pre-dated high-throughput sequencing techniques and focused on interactions between common respiratory pathobionts. (Bogaert D, et al. (2004) Lancet. 363(9424):1871-1872; Chien Y W, et al. (2013) Pediatr Infect Dis J. 32(1):72-77). Although important, much less knowledge exists about commensals that compete with these pathobionts and may be safe probiotic candidates.

### Non-Diphtheriae Corynebacterium Species are Common Commensals in the Human Respiratory Tract.

Of the more than 110 species of Corynebacterium identified to date, more than half were first cultured from human samples. The most frequent species isolated from the human respiratory tract are C. accolens, C. pseudodiphtheriticum, C. propinquum, C. striatum, and C. tuberculostearicum. (Nhan T X, et al. (2012) Diagn Microbiol Infect Dis. 74(3):236-241; Teutsch B, et al. (2017) Infection. 45(5):607-611). Except for Corynebacterium diphtheriae, the etiological agent of diphtheria, Corynebacterium spp. rarely associate with human disease and then most often in the setting of severely compromised host immunity. (Efstratiou A, et al. (1996) Rev Med Microbiol. 7(1):31-42; Spach D H, et al. (1991) Rev Infect Dis. 13(2):342-343; Morris A, et al. (1991) Rev Infect Dis. 13(5):887-892; Kiryukhina N V, et al. (2013) Probiotics Antimicrob Proteins. 5(4):233-238). The low pathogenicity of Corynebacterium spp. is a central feature that supports their further evaluation as biotherapeutics.

The Mechanisms by which Corynebacterium Spp. Colonize the Respiratory Tract and Invade Host Tissues are Poorly Defined.

Given its role in human disease, substantial work has been done to understand the pathogenesis of C. diphtheriae infection. SpaA pili mediate adherence to pharyngeal epithelial cells, while diphtheria toxin is the primary virulence factor and causes the symptoms of diphtheria. (Tauch A, et al. (2015) FEMS Microbiol Lett. 362(23):fnv185; Broadway M M, et al. (2013) J Bacteriol. 195(16):3774-3783; Gaspar A H, et al. (2006) J Bacteriol. 188(4):1526-1533; Oliveira A, et al. (2017) Front Microbiol. 8:1937). Two other species—C. ulcerans and C. pseudotuberulosis—cause primarily animal disease and were the subject of a recent detailed genomic analysis. (Tauch A, et al. (2015) FEMS Microbiol Lett. 362(23):fnv185; Oliveira A, et al. (2017) Front Microbiol. 8:1937; Sangal V, et al. (2015) BMC Genomics. 16:765; Timms V J, et al. (2018) BMC Genomics. 19(1):869; Barh D, et al. (2011) Chem Biol Drug Des. 78(1):73-84; Trost E, et al. (2011) BMC Genomics. 12(1):383; Trost E, et al. (2012) J Bacteriol. 194(12):3199-3215). Otherwise, the genetics and adherence phenotypes of commensal Corynebacterium spp., particularly human respiratory species, remain understudied.

### Corynebacterium Spp. can Inhibit Colonization by Bacterial Respiratory Pathogens.

Previous studies demonstrate that Corynebacterium spp. antagonize several important pathobionts. C. propinquum was recently recognized to inhibit the growth of staphylococci by iron restriction through siderophore production. (Stubbendieck R M, et al. (2019) Appl Environ Microbiol. 85(10)). Moreover, intranasal administration of a C. pseudodiphtheriticum strain effectively eradicated S. aureus nasal carriage in adults, demonstrating the biotherapeutic potential of Corynebacterium spp. (Kiryukhina N V, et al. (2013) Probiotics Antimicrob Proteins. 5(4):233-238) Finally, emerging data indicate that Corynebacterium spp. inhibit Sp colonization. Lower Corynebacterium abundances are observed in the nasopharynxes of Sp-colonized children. (Kelly M S, et al. (2017) Pediatr Infect Dis J. 36(9):e211-e218; Bomar L, et al. (20165) MBio. 7(1):e01725-e1815; Laufer A S, et al. (2011) MBio. 2(1):e00245-e00310). Mechanistically, Bomar et al. identified a C. accolens strain that inhibits Sp through production of a lipase that releases Sp-inhibitory free fatty acids from human skin triacylglycerols. (Bomar L, et al. (20165) MBio. 7(1):e01725-e1815). Although this study provides provocative evidence of a potential mechanism of Corynebacterium-Sp interaction, it is unclear if this lipase inhibits Sp in the presence of respiratory epithelium or contributes to population-level trends in S. pneumoniae colonization. Moreover, other mechanisms by which Corynebacterium spp. inhibit Sp colonization are likely but remain unexplored.

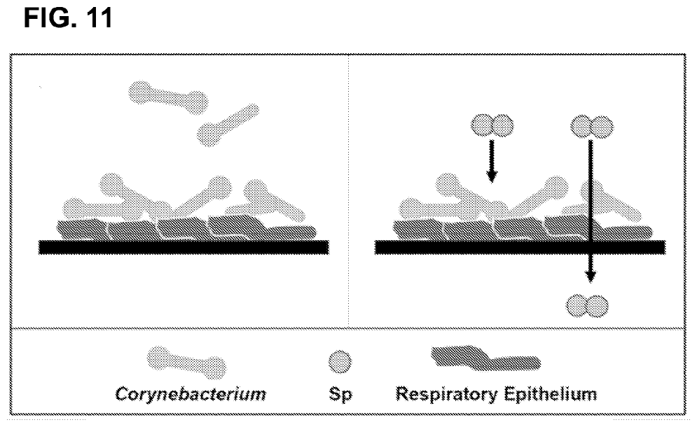

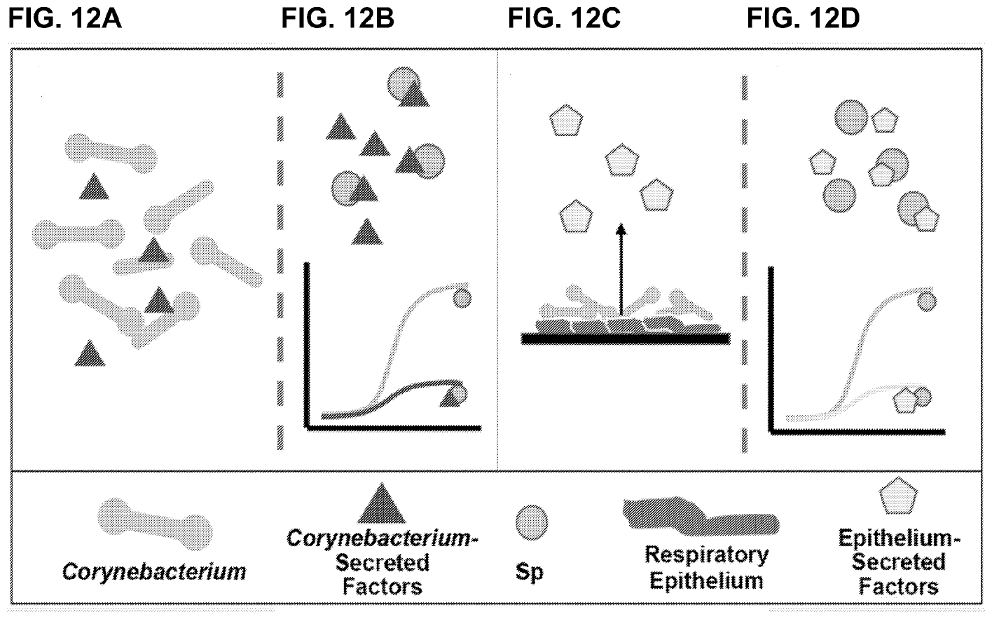

The work described herein focuses on adherence as a mechanism by which Corynebacterium spp. colonize the respiratory epithelium and competitively exclude Sp (FIG. 11A-FIG. 11B). As detailed below, the adherence of Corynebacterium strains isolated from the human respiratory tract and a non-respiratory source (skin) are measured in models for different parts of the human respiratory tract. The ability of strains isolated from the respiratory tract to inhibit respiratory epithelium adherence and invasion by Sp is evaluated. Genome-wide association analyses are used to identify genomic-phenotypic relationships for epithelium adherence. Similarly, the work described herein also focuses on non-adherence mechanisms by which Corynebacterium spp. inhibit Sp colonization (FIG. 12). Cell-free Corynebacterium supernatants are screened for secreted inhibitory factors active against Sp. Comparative proteomics are employed to identify Corynebacterium-secreted proteins that inhibit or kill Sp. Supernatants from Corynebacterium-exposed respiratory epithelium are also screened for Sp inhibition to identify strains that stimulate the release of host-derived Sp-inhibitory factors.

### Example 11

Identification Mechanisms by which Corynebacterium Spp. Adhere to Respiratory Epithelium and Inhibit Sp Colonization Through Competitive Adherence

Respiratory isolates of Corynebacteria exhibit high respiratory epithelium adherence and exclude Sp through competitive binding at the epithelial surface. Here, the key experiments include the following. First, adherence of Corynebacterium strains from the respiratory tract and skin are quantified in human respiratory epithelium tissue models. Second, whole-genome sequencing, assembly, and genome annotation are performed. Third, comparative genomics are used to identify genes that are associated with adherence to respiratory epithelium. Fourth, competitive Corynebacterium-Sp adherence experiments are performed for Corynebacterium strains that adhere to respiratory epithelium.

### Examination of Respiratory Epithelium Adherence.

Adherence by Corynebacterium to the respiratory epithelium is fundamental to colonization and persistence. Corynebacterium is expected to differentially adhere to distinct areas of the respiratory epithelium. The adherence of 250 strains from the disclosed biorepository is measured to model upper and lower respiratory epithelium using respiratory epithelium cells RMPI 2650 (human nasal carcinoma, ATCC CCL-30), Detroit 562 (human pharyngeal carcinoma; ATCC CCL-138), and A549 cells (human lung carcinoma; ATCC CCL-185), respectively, using the previously described assays (see, e.g., FIG. 8). Although immortalized, these cell lines provide a functional representation of each niche corroborated by human challenge and primary cell studies (Weight C M, et al. (2019) Nat Commun. 10(1):3060) and are amenable to measuring adherence for 250 strains with biological and technical triplicates for rigor and reproducibility. The human upper respiratory tract isolate C. accolens 05-122 (FIG. 8) is used as the control reference strain to ensure assay to assay reproducibility.

### Examination of Competitive Adherence of Sp by Corynebacterium Spp.

Corynebacterium spp. can exclude or control Sp in the human nasopharynx through competitive adherence and niche occupancy. Thus, competitive adherence assays are performed (see, for example, FIG. 8) with each of the 250 Corynebacterium strains and two Sp strains (serotype 3, ATCC 6303; serotype 6b, ATCC 700675). The rationale for testing two different Sp strains is to increase the likelihood that any competitive advantage of a Corynebacterium strain is not unique to one Sp type. The top ten strains for percent adherence are prioritized for further measurements of adherence and competitive exclusion of Sp using differentiated, polarized human primary upper respiratory nasal and pharyngeal cells (PromoCell). Differentiated, polarized primary cells are used to confirm the adherence and competition of Corynebacterium phenotypes of interest on an epithelium to more closely resembling the native normal upper respiratory epithelium.

Corynebacterium isolates can also inhibit Sp transepithelial invasion by blocking Sp adherence or independently modulating Sp endocytosis or paracellular migration. (Weight C M, et al. (2019) Nat Commun. 10(1):3060; Clarke T B, et al. (2011) Cell Host Microbe. 9(5):404-414; Zhang J R, et al. (2000) Cell. 102(6):827-837; Agarwal V, et al. (2010) J Biol Chem. 285(46):35615-35623). Detroit 562 cells are grown to confluence in 3-micron PET transwell units for ten days, measuring transepithelial resistance for polarization. (Weight C M, et al. (2019) Nat Commun. 10(1):3060). The top ten adherent Corynebacterium strains with and without the capacity for competitive adherence with Sp are added to the apical side of the epithelium (top chamber) for 30 min (MOI ˜20:1), washed, and then challenged with infant NP Sp strain 05-160, serotype 11A, at an approximate MOI of 20:1 for 1 hour. FITC-dextran (1 mg/mL; Sigma Aldrich) are added to the apical media to measure permeability. The epithelial cells are lysed and plated for Corynebacterium and Sp CFU (see, e.g., FIG. 8). Medium from the bottom chamber (containing bacteria that have crossed the epithelial cell layer) are plated and counted for bacterial CFUs to measure Sp invasion. Bottom chamber FITC dextran are measured at 488 nm (Biotek Synergy HTX). Controls 1 include no added Corynebacteria or Sp and control epithelial cells treated with 100 μM dynasore (Sigma Aldrich) and 7.5 μg/mL nystatin (Sigma Aldrich) as Sp endocytosis inhibitors. (Weight C M, et al. (2019) Nat Commun. 10(1):3060). Corynebacterium strains of interest preclude Sp from invading into the basal chamber. Biological and technical triplicates are performed.

Host Cell Viability with Corynebacterium Exposure.

The commensal Corynebacterium spp. are not expected to invoke host cellular death. However, such responses would limit strains as candidate biotherapeutics, regardless of capacity to inhibit Sp colonization. The viability of Detroit 562 and A549 cells are tested after exposure to each of the 250 Corynebacterium strains for 30 min and 120 min using ATP luciferase viability and propidium dye exclusion assays (Sigma Aldrich) for early and late-stage viability testing. Luminescence and 515 nm emission are measured for each of the assays, respectively (Biotek Synergy HTX).

### Corynebacterium Sequencing and Pan-Genome Analysis.

Notably, GenBank does not contain complete genomes for strains from any of the species of Corynebacterium that are most commonly isolated from the human respiratory tract. Therefore, for all species in the disclosed biorepository for which a complete genome is unavailable in GenBank, a representative strain is selected to generate a complete genome sequence. Extracted DNA (Lucigen Masterpure) are prepared for Illumina compatible sequencing libraries using iGenomx Riptide and short read sequenced on the Northwestern NUSeq core Illumina NovaSeq 6000 instrument (150 bp paired-end reads, 60-100× coverage). Long-read sequencing are performed on an Oxford Nanopore Minion. Genome assemblies are performed (Canu), polished (Arrow), and annotated for open-reading frames, virulence factors, and antibiotic resistance genes (DFAST; Abricate). (Koren S, et al. (2017) Genome Res. 27(5):722-736; Tanizawa Y, et al. (2018) Bioinformatics. 34(6):1037-1039; ABRicate at https://github.com/tseemann/abricate; Feldgarden M, et al. (2019) Antimicrob Agents Chemother. 63(11); Jia B, et al. (2017) Nucleic Acids Res. 45(D1):D566-D573; Chen L, et al. (2016) Nucleic Acids Res. 44(D1):D694-697). psortb are used to make in silico predictions of gene product cellular localization, which help to focus on potential surface-associated factors and predicted secreted factors. (Yu N Y, et al. (2010) Bioinformatics. 26(13):1608-1615). A pangenome analysis (with methods described herein and in FIG. 10) are performed using these new de novo assemblies and the publicly available draft and complete genomes meeting the criteria of having total base and open-reading frame counts within 2 standard deviations of the mean (currently 673 genomes classified in the genus Corynebacterium meet these criteria). Gene clusters are defined as those with pblast scores less than 10⁻⁸ and ≥75% gene coverage. Core genes are defined as those genes in 100% of all genomes. Shared accessory genes are defined as genes represented in 6-99% of the genomes. Specific genes are defined as genes in ≤5% of genomes.

### Comparative Genome Analyses.

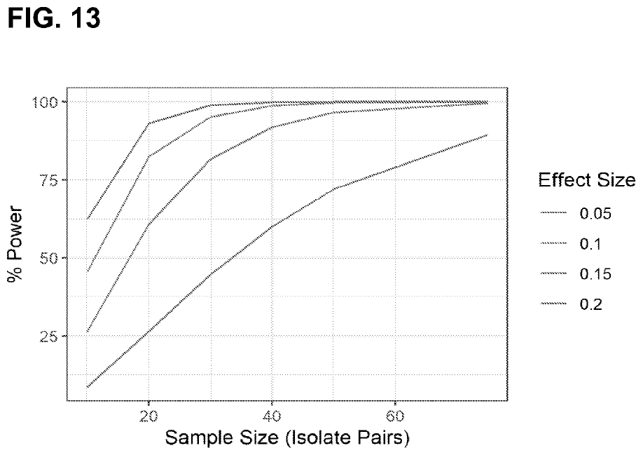

BWA is used to map short sequencing reads to the Corynebacterium pan-genome. (Li H, et al. (2009) Bioinformatics. 25(14):1754-1760). treeWAS is employed to test for associations between the gene content of Corynebacterium strains and the degree of (i) respiratory epithelium adherence, (ii) Sp inhibition through competitive adherence, (iii) inhibition of Sp invasion, and (iv) epithelial viability measures. (Collins C, et al. (2018) PLoS Comput Biol. 14(2):e1005958). Each variable is used as continuous data. treeWAS is specifically designed for microbial genome-wide association data. By combining multiple tests of association, two of which account for the phylogenetic relationship among isolates and one which is agnostic about phylogeny, it outperforms methods that fail to account for population structure and pairwise methods that can only use a subset of data because they depend on closely related pairs of isolates. Power is estimated as a function of effect size and number of related isolate pairs with different phenotypes (see FIG. 13). (Farhat M R, et al. (2014) Genome Med. 6(11)).

Comparative genomics identifies accessory genes that encode specific surface pili and adhesins that likely mediate adherence of Corynebacterium spp. to the human respiratory epithelium. Moreover, a subset of these adhesive factors correlates with competitive adherence of Sp. The presence (or absence) of these genes explains observed strain-level differences in the studied phenotypes. The results of the Sp adherence and invasion assays are used to select specific Corynebacterium strains for further study as candidate biotherapeutics.

A high degree of genetic variation across strains and species can preclude the identification of specific genes or gene variants that are associated with phenotypes using a whole-genome association approach. An alternative approach involves limiting comparative analyses to genes known to encode adhesive factors, such as pili or adhesins. To promote the inclusion of strains with varied gene content and phenotypes, culturing strains from clinical samples collected from several patient populations and ecological niches are employed. However, strains from some regions with high child pneumonia mortality (e.g., South Asia) are not currently available but can be collected through established research collaborations.

In short, integrated co-cultivation experiments, comparative genomics, and comparative proteomics localize the mechanisms and factors by which Corynebacterium strains inhibit Sp colonization, including competitive adherence, secreted inhibitors, and stimulation of the release of antimicrobial substances from the host respiratory epithelium. Employing a prioritization schema identifies many candidate genes and proteins, increasing the likelihood of identifying new mechanisms of pathogen inhibition by Corynebacteria.

### Example 12

Elucidate Non-Adherence Mechanisms by which Corynebacterium Spp. Inhibit Sp Colonization

Specific Corynebacterium strains exclude Sp through secretion of antimicrobial peptides or stimulation of release of specific defensins by host respiratory epithelium.

There are several key experiments. First, cell-free Corynebacterium supernatants are screened for Sp inhibition. Second, supernatants from Corynebacterium-exposed respiratory epithelium are screened for host-provoked Sp inhibition. Third, proteomics are used to identify bacteria and host peptides in supernatants demonstrating Sp inhibition.

This research provides substantial new genomic and phenotypic knowledge about non-diphtheriae Corynebacterium and how different species adapted to compete with the global pathogen Sp. Through newly discovered mechanisms elucidated herein by which Corynebacterium spp. colonize the respiratory tract and inhibit pathogen colonization and growth, potential biotherapeutics, including probiotics and antimicrobial factors, are identified. This research can ultimately lead to developing the first rationally-designed NP-delivered probiotics for pneumonia prevention, which can substantially impact global child mortality.

### Corynebacterium Secretome and Sp Inhibition.

In vitro cultivation assays are evaluated the hypothesis that Corynebacterium strains from the human respiratory tract secrete factors that inhibit or kill Sp (FIG. 13). Cell-free supernatants are prepared from 250 strains of Corynebacterium and added to cultures of Sp reference strains (05-122 and ATCC 700675) as in FIG. 9A-FIG. 9B. Continuous optical density (600-nm; BioTek Synergy) are measured for growth for 18 hours. As a control Sp-inhibitory medium, BHI with oleic acid (a free fatty acid that is toxic to Sp) are used as described (Bomar L, et al. (20165) MBio. 7(1):e01725-e1815). Assays are performed in biological and technical triplicate. Supernatant inhibitory peptides are identified using liquid chromatography-mass spectrometry in the Northwestern Proteomics Facility.

### Comparative Proteomic Analyses.

Mass spectra are generated on most potent inhibitory and non-inhibitory (control) supernants to identify bacterial and host proteins that mediate Sp inhibition. Spectra are analyzed using Mascot (Matrix Science) and the National Center for Biotechnology Information's non-redundant protein (nr) database. Peptide and protein identifications are validated using Scaffold (Proteome Software, Inc.). The false discovery rate for peptide-spectrum matching and protein identification are set at 10% and determined using the target-decoy search strategy. (Elias J E, et al. (2010) Methods Mol Biol. 04:55-71). Peptide identifications are accepted if established at >95.0% probability using PeptideProphet. (Nesvizhskii A I, et al. (2003) Anal Chem. 75(17):4646-4658). Protein identifications are accepted when established at >99.9% probability using PeptideProphet and contain at least two identified constituent peptides. Candidate bacterial genes are identified from these peptide sequences through analysis of the corresponding strain genome. (Camacho C, et al. (2009) BMC Bioinformatics. 10:421).

### Elicitation of an Sp-Inhibitory Host Response by Corynebacterium

Respiratory epithelial cells release innate immune response factors such as antimicrobial peptides to control the local microbiota and limit pathogen burden. (Leiva-Juirez M M, et al. (2018) Mucosal Immunol. 11(1):21-34). Whether contact-dependent interaction between Corynebacterium spp. and the respiratory epithelium promotes the expression of host antimicrobial peptides that inhibit Sp. Detroit 562 cells are co-cultured with adherent Corynebacterium strains for 6 hours. Sterile 0.2-micron filtered cell-free tissue culture supernatants are co-incubated with the Sp reference strains at a 1:1 ratio in BHI medium. A 1:1 ratio of BHI and tissue culture medium are used as a negative control and BHI with human 0-defensin-2 serve as a positive inhibition control. Growth of Sp is monitored continuously by OD600 with shaking at 37° C. for 8 hours. Peptides are identified in the Northwestern Proteomics Facility using LC/MS that are uniquely present in the inhibitory supernatants and produced by human respiratory epithelium (to differentiate from bacterial proteins). Where available commercially or possible through synthesis, recombinant factors are tested to recapitulate and validate the inhibition.

The work described herein identifies lantibiotics that are secreted by Corynebacterium strains and that inhibit Sp. In addition, Corynebacterium strains that stimulate the release of defensins from human epithelial surfaces as a mechanism of inhibition of Sp adherence and growth are identified. Collectively, the data generated herein enable the selection of Corynebacterium strains based on potency of Sp inhibition for further investigation as biotherapeutics. Alternatively, knowledge of these genetic mechanisms is used to bioengineer other commensal bacteria for use as probiotics for respiratory infection prevention. Preliminary data point towards peptide inhibitors of Sp by Corynebacterium spp but do not exclude non-peptide molecules. In the case of Sp inhibition not abrogated by proteinases or other indicators of peptide factors, non-targeted mass spectrometry is employed. Strains are also subjected to random transposition (Lucigen In Vivo Transposom Complexes) and screened for mutants with attenuation in the secreted Sp inhibition activity.

In summary, the work described herein sought to identify interspecies interactions that modify the risk of S. pneumoniae colonization during infancy and to describe development of the upper respiratory microbiome during infancy in a sub-Saharan African setting. Briefly, nasopharyngeal swabs were collected monthly (0-6 months of age) or bimonthly (6-12 months of age) from 179 mother-infant dyads in Botswana. 16S ribosomal RNA gene sequencing was used to characterize the nasopharyngeal microbiome and identified S. pneumoniae colonization using a species-specific PCR assay. Here, S. pneumoniae colonization was detected in 144 (80%) infants at a median age of 71 days and a strong negative association between the relative abundance of the bacterial genera Corynebacterium within the infant nasopharyngeal microbiome and the risk of S. pneumoniae colonization was identified. Using in vitro cultivation experiments, growth inhibition of S. pneumoniae by secreted factors from strains of several Corynebacterium species isolated from these infants was demonstrated. Finally, it was demonstrated that antibiotic exposures and the winter season are associated with a decline in the relative abundance of Corynebacterium within the nasopharyngeal microbiome, while breastfeeding was associated with an increase in the Corynebacterium relative abundance. These findings provide novel insights into the interspecies interactions that contribute to colonization resistance to S. pneumoniae and indicate that the nasopharyngeal microbiome may be a previously unrecognized mechanism by which environmental factors influence the risk of pneumococcal infections during childhood.

This work demonstrates the feasibility and the desirability of using Corynebacteria-targeted manipulation of the nasopharyngeal microbiome prevent infections caused by S. pneumoniae and other pathogenic bacteria.

What is claimed is:

1. A pharmaceutical formulation, comprising: a biotherapeutic, and at least one pharmaceutically acceptable carrier.

2. The pharmaceutical formulation of claim 1, wherein the biotherapeutic comprises a probiotic or a consortium of probiotics.

3. The pharmaceutical formulation of claim 2, further comprising a growth medium to sustain the probiotic or the consortium of probiotics.

4. The pharmaceutical formulation of claim 2, wherein the probiotic or the consortium of probiotics are lyophilized or freeze-dried.

5. The pharmaceutical formulation of claim 2, wherein the probiotic comprises at least one strain from the bacterial genus Corynebacterium.

6. The pharmaceutical formulation of claim 2, wherein the consortium of probiotics comprises one or more of at least one strain from the bacterial genus Corynebacterium, at least one strain from the bacterial genus Dolosigranulum, at least one strain from the bacterial genus Streptococcus, and at least one strain from the bacterial genus Lactobacillus.

7. The pharmaceutical formulation of claims 5-6, wherein the disclosed strain from the bacterial genus Corynebacterium comprises C. accolens, C. afermentans subsp. afermentans, C. afermentans subsp. lipophilum, C. ammoniagenes, C. amycolatum, C. appendicis, C. aquaticum, C. argentoratense, C. atypicum, C. aurimucosum, C. auris, C. bovis, C. canis, C. confusum, C. coyleae, C. diphtheriae, C. durum, C. efficiens, C. equi (now Rhodococcus equi), C. falsenii, C. flavescens, C. freiburgense, C. freneyi, C. glucuronolyticum, C. glutamicum, C. granulosum, C. haemolyticum, C. halofytica, C. kroppenstedtii, C. hansenii, C. imitans, C. jeikeium (group JK), C. kroppenstedtii, C. kutscheri, C. lipophiloflavum, C. macginleyi, C. massiliense, C. mastitidis-like, C. matruchotii, C. minutissimum, C. mucifaciens, C. mycetoides, C. ovis, C. parvum (Propionibacterium acnes), C. paurometabolum, C. pilbarense, C. propinquum, C. pseudodiphtheriticum (C. hofmannii), C. pseudotuberculosis, C. pyogenes-Trueperella pyogenes, C. pyruviciproducens, C. renale, C. resistans, C. riegelii, C. simulans, C. singular, C. spec, C. sputi, C. stationis, C. striatum, C. sundsvallense, C. tenuis, C. thomsenii, C. timonense, C. tuberculostearicum, C. tuscaniense, C. ulcerans, C. urealyticum (group D2), C. urealyticum, C. xerosis, Corynebacterium_BWA136, Corynebacterium_BWA297, Corynebacterium_DU041, Corynebacterium_DU044, or any combination thereof.

8. The pharmaceutical formulation of any one of claims 2-7, wherein the biotherapeutic comprises least 10³, 10⁴, 10⁵, 10⁶, 10⁷, 10⁸, 10⁹, 10¹⁰, 10¹¹, or 10¹² CFUs of the probiotic, or at least 10³, 10⁴, 10⁵, 10⁶, 10⁷, 10⁸, 10⁹, 10¹⁰, 10¹¹, or 10¹² CFUs of each of the probiotics in the consortium.

9. The pharmaceutical formulation of claim 6, wherein the consortium comprises the at least one strain from the bacterial genus Corynebacterium, the at least one strain from the bacterial genus Dolosigranulum, the at least one strain from the bacterial genus Streptococcus, and the at least one strain from the bacterial genus Lactobacillus in a ratio of about 1:0.01:0.01:0.01 to about 1:1:1:1.

10. The pharmaceutical formulation of any one of claims 1-9, wherein the pharmaceutical formulation is configured for nasal or nasopharyngeal administration.

11. The pharmaceutical formulation of any one of claims 1-9, wherein the pharmaceutical formulation is configured for oral administration.

12. The pharmaceutical formulation of claim 1, wherein the biotherapeutic comprises one or more factors secreted by a probiotic, or wherein the biotherapeutic comprises one or more factors secreted by a consortium of probiotics.

13. The pharmaceutical formulation of claim 12, wherein the probiotic comprises at least one strain from the bacterial genus Corynebacterium.

14. The pharmaceutical formulation of claim 12, wherein the consortium of probiotics comprises one or more of at least one strain from the bacterial genus Corynebacterium, at least one strain from the bacterial genus Dolosigranulum, at least one strain from the bacterial genus Streptococcus, and at least one strain from the bacterial genus Lactobacillus.

15. The pharmaceutical formulation of claims 13-14, wherein the disclosed strain from the bacterial genus Corynebacterium comprises C. accolens, C. afermentans subsp. afermentans, C. afermentans subsp. lipophilum, C. ammoniagenes, C. amycolatum, C. appendicis, C. aquaticum, C. argentoratense, C. atypicum, C. aurimucosum, C. auris, C. bovis, C. canis, C. confusum, C. coyleae, C. diphtheriae, C. durum, C. efficiens, C. equi (now Rhodococcus equi), C. falsenii, C. flavescens, C. freiburgense, C. freneyi, C. glucuronolyticum, C. glutamicum, C. granulosum, C. haemolyticum, C. halofytica, C. kroppenstedtii, C. hansenii, C. imitans, C. jeikeium (group JK), C. kroppenstedtii, C. kutscheri, C. lipophiloflavum, C. macginleyi, C. massiliense, C. mastitidis-like, C. matruchotii, C. minutissimum, C. mucifaciens, C. mycetoides, C. ovis, C. parvum (Propionibacterium acnes), C. paurometabolum, C. pilbarense, C. propinquum, C. pseudodiphtheriticum (C. hofmannii), C. pseudotuberculosis, C. pyogenes-Trueperella pyogenes, C. pyruviciproducens, C. renale, C. resistans, C. riegelii, C. simulans, C. singular, C. spec, C. sputi, C. stationis, C. striatum, C. sundsvallense, C. tenuis, C. thomsenii, C. timonense, C. tuberculostearicum, C. tuscaniense, C. ulcerans, C. urealyticum (group D2), C. urealyticum, C. xerosis, Corynebacterium_BWA136, Corynebacterium_BWA297, Corynebacterium_DU041, Corynebacterium_DU044, or any combination thereof.

16. A method of promoting respiratory health in a subject, the method comprising:
administering to a subject in need thereof a therapeutically effective amount of a biotherapeutic, wherein the biotherapeutic inhibits and/or prevents the growth and/or colonization of one or more pathogenic bacteria in one or more parts of the subject's respiratory system.

17. The method of claim 16, further comprise characterizing the microbiome of a biological sample.

18. The method of claim 17, wherein the microbiome comprises the nasopharyngeal microbiome, the nasal microbiome, or both.

19. The method of claim 17, wherein characterizing the microbiome comprises:
collecting a biological sample from the subject;
extracting nucleic acid from the subject's biological sample; and
sequencing the extracted nucleic acid to generate sequence data.

20. The method of claim 19, wherein the biological sample comprises a nasal swab or lavage, a nasopharyngeal swab or lavage, or a pharyngeal swab or lavage, or any combination thereof.

21. The method of claim 19, further comprising analyzing the sequence data using taxonomic classification, wherein taxonomic classification comprises performing PCR amplification.

22. The method of claim 21, wherein performing PCR amplification comprises using a pair of primers.

23. The method of claim 22, wherein the pair of primers comprises the sequence set forth in SEQ ID NO:01 and in SEQ ID NO:02, wherein the pair of primers comprises the sequence set forth in SEQ ID NO:03 and in SEQ ID NO:04, or wherein the pair of primers comprises the sequence set forth in SEQ ID NO:05 and in SEQ ID NO:06.

24. The method of any one of claims 16-23, further comprising identifying the one or more pathogenic bacteria.

25. The method of claim 24, wherein the pathogenic bacteria comprise S. pneumoniae, H. influenzae, or Moraxella catarrhalis.

26. The method of any one of claims 16-24, wherein the biotherapeutic comprises a probiotic, a consortium of probiotics, factors secreted from a probiotic, factors secreted from a consortium of probiotics, or any combination thereof, or wherein the biotherapeutic comprises a pharmaceutical formulation comprising a probiotic, a consortium of probiotics, factors secreted from a probiotic, factors secrete from a consortium of probiotics, or any combination thereof.

27. The method of claim 26, wherein the probiotic comprises at least one strain from the bacterial genus Corynebacterium.

28. The method of claim 26, wherein the consortium of probiotics comprises one or more of at least one strain from the bacterial genus Corynebacterium, at least one strain from the bacterial genus Dolosigranulum, at least one strain from the bacterial genus Streptococcus, and at least one strain from the bacterial genus Lactobacillus.

29. The method of any one of claims 27-28, wherein the disclosed strain from the bacterial genus Corynebacterium comprises C. accolens, C. afermentans subsp. afermentans, C. afermentans subsp. lipophilum, C. ammoniagenes, C. amycolatum, C. appendicis, C. aquaticum, C. argentoratense, C. atypicum, C. aurimucosum, C. auris, C. bovis, C. canis, C. confusum, C. coyleae, C. diphtheriae, C. durum, C. efficiens, C. equi (now Rhodococcus equi), C. falsenii, C. flavescens, C. freiburgense, C. freneyi, C. glucuronolyticum, C. glutamicum, C. granulosum, C. haemolyticum, C. halofytica, C. kroppenstedtii, C. hansenii, C. imitans, C. jeikeium (group JK), C. kroppenstedtii, C. kutscheri, C. lipophiloflavum, C. macginleyi, C. massiliense, C. mastitidis-like, C. matruchotii, C. minutissimum, C. mucifaciens, C. mycetoides, C. ovis, C. parvum (Propionibacterium acnes), C. paurometabolum, C. pilbarense, C. propinquum, C. pseudodiphtheriticum (C. hofmannii), C. pseudotuberculosis, C. pyogenes-Trueperella pyogenes, C. pyruviciproducens, C. renale, C. resistans, C. riegelii, C. simulans, C. singular, C. spec, C. sputi, C. stationis, C. striatum, C. sundsvallense, C. tenuis, C. thomsenii, C. timonense, C. tuberculostearicum, C. tuscaniense, C. ulcerans, C. urealyticum (group D2), C. urealyticum, C. xerosis, Corynebacterium_BWA136, Corynebacterium_BWA297, Corynebacterium_DU041, Corynebacterium_DU044, or any combination thereof.

30. The method of claim 28, wherein the therapeutically effective amount of the biotherapeutic comprises at least 10³, 10⁴, 10⁵, 10⁶, 10⁷, 10⁸, 10⁹, 10¹⁰, 10¹¹, or 10¹² CFUs of a probiotic, or wherein the therapeutically effective amount of the biotherapeutic comprises at least 10³, 10⁴, 10⁵, 10⁶, 10⁷, 10⁸, 10⁹, 10¹⁰, 10¹¹, or 10¹² CFUs of each probiotic in the consortium.

31. The method of any one of claims 16-29, further comprising administering to the subject a therapeutically effective amount of one or more anti-bacterial agents.

32. The method of any one of claim 31, wherein an anti-bacterial agent comprises a penicillin, a cephalosporin, a quinolone, an aminoglycoside, a monobactam, a carbapenem, or a macrolide.

33. The method of any one of claims 16-32, further comprising decreasing the relative abundance of the pathogenic bacteria in the subject's microbiome.

34. The method of any one of claims 16-33, wherein the subject is an adult, a child, or an infant.

35. The method of any one of claims 16-34, further comprising repeating the administering of the biotherapeutic to the subject.

36. The method of any one of claims 16-35, further comprising monitoring the subject.

37. The method of claim 36, wherein monitoring comprises monitoring the subject for the development of adverse effects.

38. The method of claim 37, wherein in the absence of adverse effects, the method further comprises continuing to treat the subject.

39. The method of any one of claims 16-38, further comprising preventing the spread and/or colonization of pathogenic bacteria to a different part of the subject's respiratory system.
